SEQUENCE LISTING (182 sequences)

SEQ ID NO: 1 - DNA, 15 bp, Artificial Sequence
  Synthetic oligonucleotide
ggcaacaaagcggcg

SEQ ID NO: 2 - DNA, 15 bp, Artificial Sequence
  Synthetic oligonucleotide
atcgccatctatttc

SEQ ID NO: 3 - DNA, 15 bp, Artificial Sequence
  Synthetic oligonucleotide
gaacgaccattagca

SEQ ID NO: 4 - DNA, 15 bp, Artificial Sequence
  Synthetic oligonucleotide
gcccagtagaacgac

SEQ ID NO: 5 - DNA, 15 bp, Artificial Sequence
  Synthetic oligonucleotide
catcgtggactggaa

SEQ ID NO: 6 - DNA, 15 bp, Artificial Sequence
  Synthetic oligonucleotide
tttcaaccgcatact

SEQ ID NO: 7 - DNA, 15 bp, Artificial Sequence
  Synthetic oligonucleotide
gagctaaacctccgt

SEQ ID NO: 8 - DNA, 15 bp, Artificial Sequence
  Synthetic oligonucleotide
gagccgccaatccat

SEQ ID NO: 9 - DNA, 15 bp, Artificial Sequence
  Synthetic oligonucleotide
aaattcctcgccctc

SEQ ID NO: 10 - DNA, 15 bp, Artificial Sequence
  Synthetic oligonucleotide
cgtagtcgtcctccc

SEQ ID NO: 11 - DNA, 15 bp, Artificial Sequence
  Synthetic oligonucleotide
ctgaaacccgccatc

SEQ ID NO: 12 - DNA, 15 bp, Artificial Sequence
  Synthetic oligonucleotide
agctcttcgataaaa

SEQ ID NO: 13 - DNA, 15 bp, Artificial Sequence
  Synthetic oligonucleotide
tcggtcagacgattg

SEQ ID NO: 14 - DNA, 15 bp, Artificial Sequence
  Synthetic oligonucleotide
ctcatcacagccgag

SEQ ID NO: 15 - DNA, 15 bp, Artificial Sequence
  Synthetic oligonucleotide
acgattctctatgcc

SEQ ID NO: 16 - DNA, 15 bp, Artificial Sequence
  Synthetic oligonucleotide
tggcatcacgattct

SEQ ID NO: 17 - DNA, 15 bp, Artificial Sequence
  Synthetic oligonucleotide
tcaattgacttatct

SEQ ID NO: 18 - DNA, 15 bp, Artificial Sequence
  Synthetic oligonucleotide
acgcccatcatattt

SEQ ID NO: 19 - DNA, 15 bp, Artificial Sequence
  Synthetic oligonucleotide
tgtctttaaaacgcc

SEQ ID NO: 20 - DNA, 15 bp, Artificial Sequence
  Synthetic oligonucleotide
ttatcaagctttgct

SEQ ID NO: 21 - DNA, 15 bp, Artificial Sequence
  Synthetic oligonucleotide
gtcatcgtaaggcag

SEQ ID NO: 22 - DNA, 15 bp, Artificial Sequence
  Synthetic oligonucleotide
ggatctttgctcgaa

SEQ ID NO: 23 - DNA, 15 bp, Artificial Sequence
  Synthetic oligonucleotide
tagtcttggatcttt

SEQ ID NO: 24 - DNA, 15 bp, Artificial Sequence
  Synthetic oligonucleotide
tgcgaaatctgtacc

SEQ ID NO: 25 - DNA, 15 bp, Artificial Sequence
  Synthetic oligonucleotide
tgaattccaccgttt

SEQ ID NO: 26 - DNA, 15 bp, Artificial Sequence
  Synthetic oligonucleotide
atcgcaatataactg

SEQ ID NO: 27 - DNA, 15 bp, Artificial Sequence
  Synthetic oligonucleotide
tgacattttctcgct

SEQ ID NO: 28 - DNA, 15 bp, Artificial Sequence
  Synthetic oligonucleotide
tcataccggcacatc

SEQ ID NO: 29 - DNA, 15 bp, Artificial Sequence
  Synthetic oligonucleotide
gtctcgtaaacttcc

SEQ ID NO: 30 - DNA, 15 bp, Artificial Sequence
  Synthetic oligonucleotide
tgatctatccgttct

SEQ ID NO: 31 - DNA, 15 bp, Artificial Sequence
  Synthetic oligonucleotide
aggtcgagccggatc

SEQ ID NO: 32 - DNA, 15 bp, Artificial Sequence
  Synthetic oligonucleotide
ccggacttgcgcctt

SEQ ID NO: 33 - DNA, 15 bp, Artificial Sequence
  Synthetic oligonucleotide
tgagagctcgaacat

SEQ ID NO: 34 - DNA, 15 bp, Artificial Sequence
  Synthetic oligonucleotide
cacggagccgcttgg

SEQ ID NO: 35 - DNA, 15 bp, Artificial Sequence
  Synthetic oligonucleotide
cattgaccagctcca

SEQ ID NO: 36 - DNA, 15 bp, Artificial Sequence
  Synthetic oligonucleotide
ggcgatatactcctt

SEQ ID NO: 37 - DNA, 15 bp, Artificial Sequence
  Synthetic oligonucleotide
caccgatgacccgcc

SEQ ID NO: 38 - DNA, 15 bp, Artificial Sequence
  Synthetic oligonucleotide
ccatcagcgtcgctc

SEQ ID NO: 39 - DNA, 15 bp, Artificial Sequence
  Synthetic oligonucleotide
ctgtttgcgctcaat

SEQ ID NO: 40 - DNA, 15 bp, Artificial Sequence
  Synthetic oligonucleotide
ggcgatgacccgctg

SEQ ID NO: 41 - DNA, 15 bp, Artificial Sequence
  Synthetic oligonucleotide
catggtcggcttctt

SEQ ID NO: 42 - DNA, 15 bp, Artificial Sequence
  Synthetic oligonucleotide
tgcgcatagactgtt

SEQ ID NO: 43 - DNA, 15 bp, Artificial Sequence
  Synthetic oligonucleotide
ggtggcgataaactc

SEQ ID NO: 44 - DNA, 15 bp, Artificial Sequence
  Synthetic oligonucleotide
atcttgtcgatgcat

SEQ ID NO: 45 - DNA, 15 bp, Artificial Sequence
  Synthetic oligonucleotide
tgttgaagcgttctt

SEQ ID NO: 46 - DNA, 15 bp, Artificial Sequence
  Synthetic oligonucleotide
cgatgttgaagcgtt

SEQ ID NO: 47 - DNA, 15 bp, Artificial Sequence
  Synthetic oligonucleotide
aagcggcctttgtcc

SEQ ID NO: 48 - DNA, 15 bp, Artificial Sequence
  Synthetic oligonucleotide
tagagttcaaagcgg

SEQ ID NO: 49 - DNA, 15 bp, Artificial Sequence
  Synthetic oligonucleotide
ccccgaaagaccacc

SEQ ID NO: 50 - DNA, 15 bp, Artificial Sequence
  Synthetic oligonucleotide
cacggatggactcga

SEQ ID NO: 51 - DNA, 15 bp, Artificial Sequence
  Synthetic oligonucleotide
agtcgatgaggttct

SEQ ID NO: 52 - DNA, 15 bp, Artificial Sequence
  Synthetic oligonucleotide
gtcagaggcgtattc

SEQ ID NO: 53 - DNA, 15 bp, Artificial Sequence
  Synthetic oligonucleotide
agcagccccgcgacc

SEQ ID NO: 54 - DNA, 15 bp, Artificial Sequence
  Synthetic oligonucleotide
gacggcgcaggcggc

SEQ ID NO: 55 - DNA, 15 bp, Artificial Sequence
  Synthetic oligonucleotide
ctgagccctcgccgg

SEQ ID NO: 56 - DNA, 15 bp, Artificial Sequence
  Synthetic oligonucleotide
ggtccacggtcaaag

SEQ ID NO: 57 - DNA, 15 bp, Artificial Sequence
  Synthetic oligonucleotide
aaaccgaaggccgag

SEQ ID NO: 58 - DNA, 15 bp, Artificial Sequence
  Synthetic oligonucleotide
aggtgcagcaagcgg

SEQ ID NO: 59 - DNA, 15 bp, Artificial Sequence
  Synthetic oligonucleotide
gtcacccggaagagt

SEQ ID NO: 60 - DNA, 15 bp, Artificial Sequence
  Synthetic oligonucleotide
agtaggattcggact

SEQ ID NO: 61 - DNA, 15 bp, Artificial Sequence
  Synthetic oligonucleotide
ccgtagaagggtgtt

SEQ ID NO: 62 - DNA, 15 bp, Artificial Sequence
  Synthetic oligonucleotide
ttcatcatgctgccg

SEQ ID NO: 63 - DNA, 15 bp, Artificial Sequence
  Synthetic oligonucleotide
ctgtgagctcgtcgg

SEQ ID NO: 64 - DNA, 15 bp, Artificial Sequence
  Synthetic oligonucleotide
ggaactgtgagctcg

SEQ ID NO: 65 - DNA, 15 bp, Artificial Sequence
  Synthetic oligonucleotide
gctcgtccttgagcg

SEQ ID NO: 66 - DNA, 15 bp, Artificial Sequence
  Synthetic oligonucleotide
tggctcgtccttgag

SEQ ID NO: 67 - DNA, 15 bp, Artificial Sequence
  Synthetic oligonucleotide
cccgttggactggat

SEQ ID NO: 68 - DNA, 15 bp, Artificial Sequence
  Synthetic oligonucleotide
cgctccgccttgatg

SEQ ID NO: 69 - DNA, 15 bp, Artificial Sequence
  Synthetic oligonucleotide
cacctgctcgcgcag

SEQ ID NO: 70 - DNA, 15 bp, Artificial Sequence
  Synthetic oligonucleotide
ggccggtgtagatga

SEQ ID NO: 71 - DNA, 15 bp, Artificial Sequence
  Synthetic oligonucleotide
tgaccgcggcctctg

SEQ ID NO: 72 - DNA, 15 bp, Artificial Sequence
  Synthetic oligonucleotide
ttgaccgcggcctct

SEQ ID NO: 73 - DNA, 15 bp, Artificial Sequence
  Synthetic oligonucleotide
taccggtctcctcgc

SEQ ID NO: 74 - DNA, 15 bp, Artificial Sequence
  Synthetic oligonucleotide
acgtacaggttgtca

SEQ ID NO: 75 - DNA, 15 bp, Artificial Sequence
  Synthetic oligonucleotide
acacgtacaggttgt

SEQ ID NO: 76 - DNA, 15 bp, Artificial Sequence
  Synthetic oligonucleotide
tcttgtcgcacgacg

SEQ ID NO: 77 - DNA, 15 bp, Artificial Sequence
  Synthetic oligonucleotide
agctcttgtcgcacg

SEQ ID NO: 78 - DNA, 15 bp, Artificial Sequence
  Synthetic oligonucleotide
ccgcacgcgtcgcac

SEQ ID NO: 79 - DNA, 15 bp, Artificial Sequence
  Synthetic oligonucleotide
tgtgcgaacttgccg

SEQ ID NO: 80 - DNA, 15 bp, Artificial Sequence
  Synthetic oligonucleotide
gctgtgcgaacttgc

SEQ ID NO: 81 - DNA, 15 bp, Artificial Sequence
  Synthetic oligonucleotide
tcgagcttgcccttg

SEQ ID NO: 82 - DNA, 15 bp, Artificial Sequence
  Synthetic oligonucleotide
agaaacggtcgatgg

SEQ ID NO: 83 - DNA, 15 bp, Artificial Sequence
  Synthetic oligonucleotide
tcggccattctcttg

SEQ ID NO: 84 - DNA, 15 bp, Artificial Sequence
  Synthetic oligonucleotide
attcggccattctct

SEQ ID NO: 85 - DNA, 15 bp, Artificial Sequence
  Synthetic oligonucleotide
cccgccggagtcagg

SEQ ID NO: 86 - DNA, 15 bp, Artificial Sequence
  Synthetic oligonucleotide
atatggaggtttagt

SEQ ID NO: 87 - DNA, 15 bp, Artificial Sequence
  Synthetic oligonucleotide
tcgaattcaccccat

SEQ ID NO: 88 - DNA, 15 bp, Artificial Sequence
  Synthetic oligonucleotide
ttcgaattcacccca

SEQ ID NO: 89 - DNA, 15 bp, Artificial Sequence
  Synthetic oligonucleotide
tgacgagatgttgtc

SEQ ID NO: 90 - DNA, 15 bp, Artificial Sequence
  Synthetic oligonucleotide
tagctgacgagatgt

SEQ ID NO: 91 - DNA, 15 bp, Artificial Sequence
  Synthetic oligonucleotide
agtttcacgacttgc

SEQ ID NO: 92 - DNA, 15 bp, Artificial Sequence
  Synthetic oligonucleotide
ccgtagaagggtgtt

SEQ ID NO: 93 - DNA, 15 bp, Artificial Sequence
  Synthetic oligonucleotide
ttcatcatgctgccg

SEQ ID NO: 94 - DNA, 15 bp, Artificial Sequence
  Synthetic oligonucleotide
ctgtgagctcgtcgg

SEQ ID NO: 95 - DNA, 15 bp, Artificial Sequence
  Synthetic oligonucleotide
ggaactgtgagctcg

SEQ ID NO: 96 - DNA, 15 bp, Artificial Sequence
  Synthetic oligonucleotide
gctcgtccttgagcg

SEQ ID NO: 97 - DNA, 15 bp, Artificial Sequence
  Synthetic oligonucleotide
tggctcgtccttgag

SEQ ID NO: 98 - DNA, 15 bp, Artificial Sequence
  Synthetic oligonucleotide
cccgttggactggat

SEQ ID NO: 99 - DNA, 15 bp, Artificial Sequence
  Synthetic oligonucleotide
cgctccgccttgatg

SEQ ID NO: 100 - DNA, 15 bp, Artificial Sequence
  Synthetic oligonucleotide
cacctgctcgcgcag

SEQ ID NO: 101 - DNA, 15 bp, Artificial Sequence
  Synthetic oligonucleotide
ggccggtgtagatga

SEQ ID NO: 102 - DNA, 15 bp, Artificial Sequence
  Synthetic oligonucleotide
tgaccgcggcctctg

SEQ ID NO: 103 - DNA, 15 bp, Artificial Sequence
  Synthetic oligonucleotide
ttgaccgcggcctct

SEQ ID NO: 104 - DNA, 15 bp, Artificial Sequence
  Synthetic oligonucleotide
taccggtctcctcgc

SEQ ID NO: 105 - DNA, 15 bp, Artificial Sequence
  Synthetic oligonucleotide
acgtacaggttgtca

SEQ ID NO: 106 - DNA, 15 bp, Artificial Sequence
  Synthetic oligonucleotide
acacgtacaggttgt

SEQ ID NO: 107 - DNA, 15 bp, Artificial Sequence
  Synthetic oligonucleotide
tcttgtcgcacgacg

SEQ ID NO: 108 - DNA, 15 bp, Artificial Sequence
  Synthetic oligonucleotide
agctcttgtcgcacg

SEQ ID NO: 109 - DNA, 15 bp, Artificial Sequence
  Synthetic oligonucleotide
ccgcacgcgtcgcac

SEQ ID NO: 110 - DNA, 15 bp, Artificial Sequence
  Synthetic oligonucleotide
tgtgcgaacttgccg

SEQ ID NO: 111 - DNA, 15 bp, Artificial Sequence
  Synthetic oligonucleotide
gctgtgcgaacttgc

SEQ ID NO: 112 - DNA, 15 bp, Artificial Sequence
  Synthetic oligonucleotide
tcgagcttgcccttg

SEQ ID NO: 113 - DNA, 15 bp, Artificial Sequence
  Synthetic oligonucleotide
agaaacggtcgatgg

SEQ ID NO: 114 - DNA, 15 bp, Artificial Sequence
  Synthetic oligonucleotide
tcggccattctcttg

SEQ ID NO: 115 - DNA, 15 bp, Artificial Sequence
  Synthetic oligonucleotide
attcggccattctct

SEQ ID NO: 116 - DNA, 15 bp, Artificial Sequence
  Synthetic oligonucleotide
cccgccggagtcagg

SEQ ID NO: 117 - DNA, 15 bp, Artificial Sequence
  Synthetic oligonucleotide
atatggaggtttagt

SEQ ID NO: 118 - DNA, 15 bp, Artificial Sequence
  Synthetic oligonucleotide
tcgaattcaccccat

SEQ ID NO: 119 - DNA, 15 bp, Artificial Sequence
  Synthetic oligonucleotide
ttcgaattcacccca

SEQ ID NO: 120 - DNA, 15 bp, Artificial Sequence
  Synthetic oligonucleotide
tgacgagatgttgtc

SEQ ID NO: 121 - DNA, 15 bp, Artificial Sequence
  Synthetic oligonucleotide
tagctgacgagatgt

SEQ ID NO: 122 - DNA, 15 bp, Artificial Sequence
  Synthetic oligonucleotide
agtttcacgacttgc

SEQ ID NO: 123 - DNA, 15 bp, Artificial Sequence
  Synthetic oligonucleotide
ggcaacaaagcggcg

SEQ ID NO: 124 - DNA, 15 bp, Artificial Sequence
  Synthetic oligonucleotide
atcgccatctatttc

SEQ ID NO: 125 - DNA, 15 bp, Artificial Sequence
  Synthetic oligonucleotide
gaacgaccattagca

SEQ ID NO: 126 - DNA, 15 bp, Artificial Sequence
  Synthetic oligonucleotide
gcccagtagaacgac

SEQ ID NO: 127 - DNA, 15 bp, Artificial Sequence
  Synthetic oligonucleotide
catcgtggactggaa

SEQ ID NO: 128 - DNA, 15 bp, Artificial Sequence
  Synthetic oligonucleotide
tttcaaccgcatact

SEQ ID NO: 129 - DNA, 15 bp, Artificial Sequence
  Synthetic oligonucleotide
gagctaaacctccgt

SEQ ID NO: 130 - DNA, 15 bp, Artificial Sequence
  Synthetic oligonucleotide
gagccgccaatccat

SEQ ID NO: 131 - DNA, 15 bp, Artificial Sequence
  Synthetic oligonucleotide
aaattcctcgccctc

SEQ ID NO: 132 - DNA, 15 bp, Artificial Sequence
  Synthetic oligonucleotide
cgtagtcgtcctccc

SEQ ID NO: 133 - DNA, 15 bp, Artificial Sequence
  Synthetic oligonucleotide
ctgaaacccgccatc

SEQ ID NO: 134 - DNA, 15 bp, Artificial Sequence
  Synthetic oligonucleotide
agctcttcgataaaa

SEQ ID NO: 135 - DNA, 15 bp, Artificial Sequence
  Synthetic oligonucleotide
tcggtcagacgattg

SEQ ID NO: 136 - DNA, 15 bp, Artificial Sequence
  Synthetic oligonucleotide
ctcatcacagccgag

SEQ ID NO: 137 - DNA, 15 bp, Artificial Sequence
  Synthetic oligonucleotide
acgattctctatgcc

SEQ ID NO: 138 - DNA, 15 bp, Artificial Sequence
  Synthetic oligonucleotide
tggcatcacgattct

SEQ ID NO: 139 - DNA, 15 bp, Artificial Sequence
  Synthetic oligonucleotide
tcaattgacttatct

SEQ ID NO: 140 - DNA, 15 bp, Artificial Sequence
  Synthetic oligonucleotide
acgcccatcatattt

SEQ ID NO: 141 - DNA, 15 bp, Artificial Sequence
  Synthetic oligonucleotide
tgtctttaaaacgcc

SEQ ID NO: 142 - DNA, 15 bp, Artificial Sequence
  Synthetic oligonucleotide
ttatcaagctttgct

SEQ ID NO: 143 - DNA, 15 bp, Artificial Sequence
  Synthetic oligonucleotide
gtcatcgtaaggcag

SEQ ID NO: 144 - DNA, 15 bp, Artificial Sequence
  Synthetic oligonucleotide
ggatctttgctcgaa

SEQ ID NO: 145 - DNA, 15 bp, Artificial Sequence
  Synthetic oligonucleotide
tagtcttggatcttt

SEQ ID NO: 146 - DNA, 15 bp, Artificial Sequence
  Synthetic oligonucleotide
tgcgaaatctgtacc

SEQ ID NO: 147 - DNA, 15 bp, Artificial Sequence
  Synthetic oligonucleotide
tgaattccaccgttt

SEQ ID NO: 148 - DNA, 15 bp, Artificial Sequence
  Synthetic oligonucleotide
atcgcaatataactg

SEQ ID NO: 149 - DNA, 15 bp, Artificial Sequence
  Synthetic oligonucleotide
tgacattttctcgct

SEQ ID NO: 150 - DNA, 15 bp, Artificial Sequence
  Synthetic oligonucleotide
tcataccggcacatc

SEQ ID NO: 151 - DNA, 15 bp, Artificial Sequence
  Synthetic oligonucleotide
gtctcgtaaacttcc

SEQ ID NO: 152 - DNA, 15 bp, Artificial Sequence
  Synthetic oligonucleotide
tgatctatccgttct

SEQ ID NO: 153 - DNA, 15 bp, Artificial Sequence
  Synthetic oligonucleotide
aggtcgagccggatc

SEQ ID NO: 154 - DNA, 15 bp, Artificial Sequence
  Synthetic oligonucleotide
ccggacttgcgcctt

SEQ ID NO: 155 - DNA, 15 bp, Artificial Sequence
  Synthetic oligonucleotide
tgagagctcgaacat

SEQ ID NO: 156 - DNA, 15 bp, Artificial Sequence
  Synthetic oligonucleotide
cacggagccgcttgg

SEQ ID NO: 157 - DNA, 15 bp, Artificial Sequence
  Synthetic oligonucleotide
cattgaccagctcca

SEQ ID NO: 158 - DNA, 15 bp, Artificial Sequence
  Synthetic oligonucleotide
ggcgatatactcctt

SEQ ID NO: 159 - DNA, 15 bp, Artificial Sequence
  Synthetic oligonucleotide
caccgatgacccgcc

SEQ ID NO: 160 - DNA, 15 bp, Artificial Sequence
  Synthetic oligonucleotide
ccatcagcgtcgctc

SEQ ID NO: 161 - DNA, 15 bp, Artificial Sequence
  Synthetic oligonucleotide
ctgtttgcgctcaat

SEQ ID NO: 162 - DNA, 15 bp, Artificial Sequence
  Synthetic oligonucleotide
ggcgatgacccgctg

SEQ ID NO: 163 - DNA, 15 bp, Artificial Sequence
  Synthetic oligonucleotide
catggtcggcttctt

SEQ ID NO: 164 - DNA, 15 bp, Artificial Sequence
  Synthetic oligonucleotide
tgcgcatagactgtt

SEQ ID NO: 165 - DNA, 15 bp, Artificial Sequence
  Synthetic oligonucleotide
ggtggcgataaactc

SEQ ID NO: 166 - DNA, 15 bp, Artificial Sequence
  Synthetic oligonucleotide
atcttgtcgatgcat

SEQ ID NO: 167 - DNA, 15 bp, Artificial Sequence
  Synthetic oligonucleotide
tgttgaagcgttctt

SEQ ID NO: 168 - DNA, 15 bp, Artificial Sequence
  Synthetic oligonucleotide
cgatgttgaagcgtt

SEQ ID NO: 169 - DNA, 15 bp, Artificial Sequence
  Synthetic oligonucleotide
aagcggcctttgtcc

SEQ ID NO: 170 - DNA, 15 bp, Artificial Sequence
  Synthetic oligonucleotide
tagagttcaaagcgg

SEQ ID NO: 171 - DNA, 15 bp, Artificial Sequence
  Synthetic oligonucleotide
ccccgaaagaccacc

SEQ ID NO: 172 - DNA, 15 bp, Artificial Sequence
  Synthetic oligonucleotide
cacggatggactcga

SEQ ID NO: 173 - DNA, 15 bp, Artificial Sequence
  Synthetic oligonucleotide
agtcgatgaggttct

SEQ ID NO: 174 - DNA, 15 bp, Artificial Sequence
  Synthetic oligonucleotide
gtcagaggcgtattc

SEQ ID NO: 175 - DNA, 15 bp, Artificial Sequence
  Synthetic oligonucleotide
agcagccccgcgacc

SEQ ID NO: 176 - DNA, 15 bp, Artificial Sequence
  Synthetic oligonucleotide
gacggcgcaggcggc

SEQ ID NO: 177 - DNA, 15 bp, Artificial Sequence
  Synthetic oligonucleotide
ctgagccctcgccgg

SEQ ID NO: 178 - DNA, 15 bp, Artificial Sequence
  Synthetic oligonucleotide
ggtccacggtcaaag

SEQ ID NO: 179 - DNA, 15 bp, Artificial Sequence
  Synthetic oligonucleotide
aaaccgaaggccgag

SEQ ID NO: 180 - DNA, 15 bp, Artificial Sequence
  Synthetic oligonucleotide
aggtgcagcaagcgg

SEQ ID NO: 181 - DNA, 15 bp, Artificial Sequence
  Synthetic oligonucleotide
gtcacccggaagagt

SEQ ID NO: 182 - DNA, 15 bp, Artificial Sequence
  Synthetic oligonucleotide
agtaggattcggact

(19) United States
(12) Patent Application Publication
(10) Pub. No.: US 2016/0201063 A1
(43) Pub. Date: Jul. 14, 2016

(54) EPIGENETIC REGULATORS OF FRATAXIN

(71) Applicant: RANA THERAPEUTICS, INC., Cambridge, MA (US)

(72) Inventor: Fatih Ozsolak, Boston, MA (US)

(73) Assignee: RaNA Therapeutics, Inc., Cambridge, MA (US)

(21) Appl. No.: 14/911,836

(22) PCT Filed: Aug. 15, 2014
(86) PCT No.: PCT/US2014/051258

§ 371 Date: Feb. 12, 2016

Related U.S. Application Data
(60) Provisional application No. 62/010,427, filed on Jun. 10, 2014.
(60) Provisional application No. 61/866,830, filed on Aug. 16, 2013.

(51) Int. Cl. C12N 15/113

(52) U.S. Cl. CPC C12N 15/1137; C12N 2310/341; C12N 2310/14

(57) ABSTRACT

Provided herein are methods for increasing Frataxin (FXN) expression that involve targeting or expressing regulatory factors that modulate the epigenetic state of FXN genes. Also provided herein are methods for increasing FXN expression using inhibitors of a negative epigenetic regulator of FXN. Compositions and methods for treating Friedrich's ataxia are also provided.

### CROSS-REFERENCE TO RELATED APPLICATIONS

This application claims the benefit under 35 U.S.C. §119(e) of U.S. Provisional Application No. 62/010,427, entitled “EPIGENETIC REGULATORS OF FRATAXIN”, filed Jun. 10, 2014 and of U.S. Provisional Application No. 61/866,830, entitled “EPIGENETIC REGULATORS OF FRATAXIN”, filed Aug. 16, 2013, the contents of each of which are incorporated herein by reference in its entirety.

### FIELD OF THE INVENTION

The invention relates in part to compositions and methods for modulating gene expression.

### BACKGROUND OF THE INVENTION

Friedreich's ataxia (FRDA) is a rare recessive inherited disease characterized by progressive degeneration of the spinal cord and peripheral nerve tissue. Symptoms resulting from this nervous system damage include muscle weakness, loss of coordination, vision and hearing impairment, speech problems, scoliosis, diabetes, and several heart disorders. Symptoms typically begin between ages of 5 and 15 years and first present as difficulty walking (gait ataxia). As the disease progresses, other symptoms develop, such as speech slurring, hearing loss, and vision loss. Various forms of heart disease often accompany FRDA, including hypertrophic cardiomyopathy, myocardial fibrosis, and cardiac failure. Approximately, ten percent of those affected by FRDA develop diabetes. Symptom progression varies between individuals, but generally within 10 to 20 years from disease onset, the person is wheelchair bound and may eventually become completely incapacitated. FRDA can lead to early death, often as a result of heart disease associated with FRDA. Reduced expression of Frataxin (FXN) is thought to cause Friedreich's ataxia (FRDA).

### SUMMARY OF THE INVENTION

According to some aspects of the invention certain regulatory factors have been identified that modulate expression of FXN in cells. Both negative and positive regulators of FXN expression have been discovered. In some embodiments, regulatory factors disclosed herein modulate FXN expression by modulating the epigenetic state of FXN genes. In some embodiments, inhibiting expression of a negative regulator of FXN results increased expression of FXN in cells, e.g., cells from a patient with FRDA. In other embodiments, inducing expression of a positive regulator of FXN results in increased expression of FXN in cells, e.g., cells from a patient with FRDA. Thus, in certain aspects, the invention provides methods and compositions that are useful for upregulating FXN in a cell. Accordingly, in some embodiments, methods and compositions provided herein are useful for the treatment and/or prevention (e.g., reducing the risk or delaying the onset) of FRDA.

Aspects of the invention relate to methods for increasing FXN expression in a cell. In some embodiments, the methods involve delivering to a cell an oligonucleotide that inhibits expression or activity of a negative epigenetic regulator of FXN, thereby increasing FXN expression in the cell. In some embodiments, prior to delivering the oligonucleotide, the cell has a lower level of FXN expression compared to an appropriate control level of FXN expression. In some embodiments, prior to delivering the oligonucleotide, the cell has a higher level of histone H3 K27 or K9 methylation at the FXN gene compared with an appropriate control level of histone H3 K27 or K9 methylation. In some embodiments, the cell comprises an FXN gene encoding in its first intron a GAA repeat of between 10-2000 units. In some embodiments, the cell is obtained from or present in a subject having Friedreich's ataxia. In some embodiments, presence of the oligonucleotide in the cell results in decreased levels of mRNA of the negative epigenetic regulator of FXN. In some embodiments, the appropriate control is a level of FXN in a cell from a subject or in cells from a population of subjects that do not have Friedreich's ataxia.

In some embodiments, the oligonucleotide comprises a sequence as set for in Table 4. In some embodiments, the oligonucleotide comprises a sequence as set for in Table 12. In some embodiments, the oligonucleotide is a gapmer, a mixmer, an siRNA, a single stranded RNA, a single stranded DNA, an aptamer, or a ribozyme. In some embodiments, the oligonucleotide comprises at least one modified nucleotide or internucleoside linkage. In some embodiments, the oligonucleotide is a single stranded oligonucleotide. In some embodiments, the single stranded oligonucleotide comprises the sequence 5′-X-Y-Z-3′, wherein X comprises 1-5 modified nucleotides, Y comprises at least 6 unmodified nucleotides, and Z comprises 1-5 modified nucleotides. In some embodiments, the X comprises 1-5 LNAs, Y comprises at least 6 DNAs, and Z comprises 1-5 LNAs.

In some embodiments, the negative epigenetic regulator of FXN is a component of a histone H2A acetylation pathway, a NuA4 histone acetyltransferase complex, a protein amino acid acetylation pathway, a histone acetylation pathway, a protein amino acid acylation pathway, a H4/H2A histone acetyltransferase complex, a nucleotide binding pathway, a histone H4 acetylation pathway, a histone acetyltransferase complex, or an insulin receptor substrate binding pathway. In some embodiments, the component of the histone H2A acetylation pathway is MEAF6, YEATS4, ACTL6A, or DMAP1. In some embodiments, the component of the NuA4 histone acetyltransferase complex is MEAF6, YEATS4, ACTL6A, or DMAP1. In some embodiments, the component of the protein amino acid acetylation pathway is KAT2A, MEAF6, YEATS4, TADA3, ACTL6A, or DMAP1. In some embodiments, the component of the histone acetylation pathway is KAT2A, MEAF6, YEATS4, TADA3, ACTL6A, or DMAP1. In some embodiments, the component of the protein amino acid acylation pathway is KAT2A, MEAF6, YEATS4, TADA3, ACTL6A, or DMAP1. In some embodiments, the component of the H4/H2A histone acetyltransferase complex is MEAF6, YEATS4, ACTL6A, or DMAP1. In some embodiments, the component of the nucleotide binding pathway is MEF2D, PRKDC, IDH1, ACTL6A, JAK2, CFTR, SPEN, or PRKCD. In some embodiments, the component of the histone H4 acetylation pathway is MEAF6, YEATS4, ACTL6A, or DMAP1. In some embodiments, the component of the histone acetyltransferase complex is KAT2A, MEAF6, YEATS4, TADA3, ACTL6A, or DMAP1. In some embodiments, the component of the insulin receptor substrate binding pathway is JAK2 or PRKCD.

In some embodiments, the negative epigenetic regulator of FXN is TNFSF9, JUND, HIC1, PRKCD, JAK2, EID1, CFTR, TADA3, MYBL2, KAT2A, IDH1, SUMO1, SPEN, PRKDC, KIR2DL4, APC, MEF2D, a component of the NuA4 Histone Acetyltransferase Complex, or a histone-lysine N-methyltransferase.

In some embodiments, the negative epigenetic regulator of FXN is a component of the NuA4 Histone Acetyltransferase Complex. In some embodiments, the component of the NuA4 Histone Acetyltransferase Complex is YEATS4, Eaf1, TRRAP, P400, EPC1, DMAP1, Tip60, MRG15, MRGX, MORF4, ACTB, ACTL6A, ING1, ING2, ING3, ING4, ING5, RUVBL1, RUVBL2, AF9, ENL, or MEAF6. In some embodiments, the component of the NuA4 Histone Acetyltransferase Complex is YEATS4, ACTL6A, DMAP1, or MEAF6. In some embodiments, the component of the NuA4 Histone Acetyltransferase Complex is YEATS4.

In some embodiments, the negative epigenetic regulator of FXN is a histone-lysine N-methyltransferase. In some embodiments, the histone-lysine N-methyltransferase is SUV39H1, SUV39H2, SETDB1, PRDM2, G9A and EHMT1. In some embodiments, the histone-lysine N-methyltransferase is SUV39H1.

In some embodiments, the negative epigenetic regulator of FXN is YEATS4, HIC1, JUND, TNFSF9, PRKCD, KAT2A, JAK2, IDH1, EID1, or ACTL6A.

In some embodiments, the negative epigenetic regulator of FXN is the product of a gene listed in Table 6 and/or 9 that has a fold change greater than 1.25.

In some embodiments, the method further comprises: delivering to the cell a second oligonucleotide. In some embodiments, the second oligonucleotide inhibits expression or activity of a second negative epigenetic regulator of FXN. In some embodiments, the second negative epigenetic regulator of FXN is TNFSF9, JUND, HIC1, PRKCD, JAK2, EID1, CFTR, TADA3, MYBL2, KAT2A, IDH1, SUMO1, SPEN, PRKDC, KIR2DL4, APC, MEF2D, a component of the NuA4 Histone Acetyltransferase Complex, or a histone-lysine N-methyltransferase.

According to some aspects of the invention methods for increasing FXN expression in a cell are provided that involve delivering to a cell an expression vector that is engineered to express a positive epigenetic regulator of FXN, thereby increasing FXN expression in the cell. In some embodiments, prior to delivering the expression vector, the cell has a lower level of FXN expression compared to an appropriate control level of FXN expression. According to some aspects of the invention methods for increasing FXN expression in a cell are provided that involve expressing a exogenous positive epigenetic regulator of FXN. In some embodiments, the positive epigenetic regulator of FXN is the product of a gene listed in Table 6 and/or 9 that has a fold change less than or equal to 1.0, 0.90, 0.85, 0.80, 0.75, or 0.50.

According to some aspects of the invention, oligonucleotides are provided that comprise a sequence as set forth in Table 4 or Table 12. In some embodiments, the oligonucleotide comprises at least one modified nucleotide or internucleoside linkage. In some embodiments, the oligonucleotide is 50 nucleotides or fewer in length. In some embodiments, the oligonucleotide consists of a sequence as set forth in Table 4. In some embodiments, the oligonucleotide consists of a sequence as set forth in Table 12.

The details of one or more embodiments of the invention are set forth in the description below. Other features or advantages of the present invention will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a graph depicting epigenetic siRNA screen fold change distribution.

FIG. 2 is a table depicting the siRNA Screening Results. “FXN downregulating genes” are genes for which reduced expression results in downregulation of FXN. “FXN upregulating genes” are genes for which reduced expression results in upregulation of FXN

FIG. 3A is a table depicting the siRNA data related to the NuA4 Histone Acetyltransferase Complex.

FIG. 3B is a graph depicting that knockdown of Suv39H1 resulted in upregulation of FXN.

FIGS. 4A and 4B shows a screen of 80 epigenetic inhibitors from a epigenetics screening library using GM03816 FRDA diseased fibroblasts (FIG. 4A; actual data in Table 10) and GM0321 normal fibroblasts (FIG. 4B; actual data in Table 11). FXN RNA levels are indicated on the y-axis and the inhibitors used at both 1 μM and 5 μM are shown on the x-axis.

FIGS. 5A-5E shows treatment of human FRDA diseased cell lines and Sarsero FXN mouse-model derived fibroblasts with a histone lysine methyltransferase inhibitor (HLMi). The Sarsero mouse model was generated by inserting the diseased human FXN gene with GAA-repeated into mouse genome. RQ: FXN RNA quantity in compound treated cells relative to untreated cells. FIG. 5A shows GM03816 cells after 2 days of treatment with the HLMi at the indicated concentration; FIG. 5B shows GM03816 cells after 3 days of treatment with the HLMi at the indicated concentration; FIG. 5C shows GM04078 cells after 3 days of treatment with the HLMi at the indicated concentration; FIG. 5D shows Sarsero fibroblasts after 3 days of treatment with the HLMi at the indicated concentration (mouse FXN expression); FIG. 5E shows Sarsero fibroblasts 3 day treatment with the HLMi at the indicated concentration (human FXN expression).

FIG. 6 shows a western blot to detect FXN protein upregulation in human FRDA diseased cell lines GM03816 and GM04078 following 3 days of treatment with a HLMi at various concentrations (5 μM, 2.5 μM, 1.25 μM). Results from control cells treated with DMSO and without inhibitor treatment are also shown.

FIGS. 7A and B are a series of graphs showing FXN mRNA levels in cells treated with gapmers for human JUND, YEATS4, HIC1, ACTL6A, EID1, IDH1, TNFSF9, JAK2, KAT2A or PRKCD; blank columns are untreated.

FIG. 8 is a photograph of a Western blot showing FXN protein levels in cells treated with gapmers for ACTL6A, JUND, PRKCD, and YEATS4.

FIG. 9 is a graph showing FXN mRNA levels in differentiated myotubes treated with various gapmers for ACTL6A, EID1, HIC1, JUND, KAT2A, PRKCD, and YEATS4.

FIGS. 10A-D are a series of graphs showing enrichment in the FXN gene locus of H3K27me3 and H3K9me3 (10A and 10B), Tip60 (10C), or SUV39H1 (10D) in diseased cell lines compared to normal cells.

FIGS. 11A and 11B are a series of graphs showing showing enrichment in the FXN gene locus of G9a (FIG. 11A) and IgG (FIG. 11B) in diseased cell lines compared to normal cells.

### DETAILED DESCRIPTION OF THE INVENTION

In some embodiments, regulatory factors disclosed herein modulate FXN expression by controlling the epigenetic state of FXN genes. In some embodiments, methods and compositions are provided that induce or enhance expression of FXN by decreasing expression or function of one or more negative epigenetic regulators of FXN. In some embodiments, this induced or enhanced expression of FXN is believed to result from a change in the chromatin state of the FXN gene, e.g., a decreased level of histone H3 K27 or K9 methylation at the FXN gene. In other aspects of the invention methods for inducing expression of a positive regulator of FXN may be used to induce or enhance expression of FXN. Here again, in some embodiments, this induced or enhanced expression of FXN is believed to result from a change in the chromatin state of the FXN gene, e.g., a decreased level of histone H3 K27 or K9 methylation at the FXN gene.

As used herein, the term “FXN gene” refers to a genomic region that encodes FXN protein and/or controls the transcription of FXN mRNA. Thus, the term encompasses coding sequences and exons as well as any non-coding elements, e.g., promoters, enhancers, silencers, introns, and 5′ and 3′ untranslated regions. An FXN gene may include flanking sequences 5′ and/or 3′ to a known annotated FXN open reading frame, e.g., 1 Kb, 2 Kb, 3 Kb, 4 Kb, 5 Kb, 6 Kb, 7 Kb, 8 Kb, 9 Kb, or 10 Kb or more flanking the 5′ and/or 3′ end of a known annotated FXN open reading frame. In some embodiments, a FXN gene may be a human FXN gene (see, e.g., NCBI Gene ID: 2395, located on chromosome 9). In some embodiments, a FXN gene may be a corresponding homolog of a FXN gene in a different species (e.g., a mouse FXN encoded by a mouse FXN gene such as NCBI Gene ID: 14297).

### Negative Epigenetic Regulators of FXN

As used herein, a “negative epigenetic regulator” is a regulatory factor (e.g., regulatory protein) that promotes the formation or maintenance of heterochromatin, and/or that inhibits the formation or maintenance of euchromatin. In some embodiments, a negative epigenetic regulator inhibits or reduces FXN expression either directly or indirectly. In some embodiments, negative epigenetic regulators mediate reduction or silencing of FXN expression though an epigenetic mechanism, e.g., though heterochromatin formation at or near the FXN gene. Accordingly, in some embodiments, when the expression level of a negative epigenetic regulator of FXN is reduced (e.g., by contacting a cell with an appropriate oligonucleotide as described herein), FXN expression is upregulated.

Without wishing to be bound by theory, it is believed that in some embodiments the heterochromatin formation at the FXN gene can be reversed, in part or in whole, by reducing the expression of one or more negative epigenetic regulators of FXN, thereby causing upregulation of FXN expression. Heterochromatin formation can be measured using any method known in the art, e.g., using an immunoassay to detect methylation patterns at or near the FXN gene. For example, levels of mono-, di- and tri-methylation of histone H3 at lysine 27 and/or lysine 9 may be measured at or near the FXN gene. An increase in these types of methylation may indicate the presence of heterochromatin in some embodiments.

Negative epigenetic regulators of FXN may act directly on the FXN gene, e.g., by catalyzing methylation of a histone, or indirectly, e.g., by forming a complex with or activating other proteins that are involved in epigenetic modification of the FXN gene. Examples of negative epigenetic regulators of FXN are provided in Tables 1 and 7. The gene ID and transcript ID for each gene are provided, which can be used to identify any gene, mRNA transcript, and protein sequences by querying the NCBI (National Center for Biotechnology Information) Gene database.

In some embodiments, a negative epigenetic regulator of FXN is the product of a gene listed in Table 6 and/or 9 that has a fold change greater than 1. In some embodiments, a negative epigenetic regulator of FXN is the product of a gene listed in Table 6 and/or 9 that has a fold change greater than 1.5. In some embodiments, a negative epigenetic regulator of FXN is the product of a gene listed in Table 6 and/or 9 that has a fold change greater than 1.75. In some embodiments, a negative epigenetic regulator of FXN is the product of a gene listed in Table 6 and/or 9 that has a fold change greater than 2. In some embodiments, a negative epigenetic regulator of FXN is the product of a gene listed in Table 6 and/or 9 that has a fold change greater than 2.5.

In some embodiments, one or more chromatin markers may be evaluated to assess the chromatin status of an FXN gene. For example, Histone H4 K₂O trimethylation may be used as a marker to indicate heterochromatin. Presence of HP1, SUV39 and/or other similar proteins may also be used to detect presence of heterochromatin at the FXN gene. Other suitable markers may be used to assess chromatin status of an FXN gene.

TABLE 1
Negative epigenetic regulators of FXN

| | | | | Mus musculus | |
|---|---|---|---|---|---|
| | | Homo sapiens | | Gene | |
| GENE | ALIASES | Gene ID | Transcript IDs | ID | Transcript ID |
| YEATS4 | 4930573H17Rik, | 8089 | NM_006530.2 | 64050 | NM_026570.4 |
| | B230215M10Rik, | | | | |
| | GAS41, NUBI-1, YAF9 | | | | |
| TNFSF9 | 4-1BB-L, CD137L | 8744 | NM_003811.3 | 21950 | NM_009404.3 |
| JUND | AP-1 | 3727 | NM_005354.4 | 6478 | NM_010592.4 |
| HIC1 | ZBTB29, ZNF901, hic-1 | 3090 | NM_001098202.1 | 15248 | NM_001098203.1 |
| | | | NM_006497.3 | | NM_010430.2 |
| PRKCD | MAY1, PKCD, nPKC- | 5580 | NM_006254.3 | 8753 | NM_011103.3 |
| | delta | | NM_212539.1 | | |
| ACTL6A | ACTL6, ARPN-BETA, | 86 | NM_177989.2 | 56456 | NM_019673.2 |
| | Arp4, BAF53A, | | NM_178042.2 | | |
| | INO80K | | NM_004301.3 | | |
| JAK2 | JTK10, THCYT3 | 3717 | NM_004972.3 | 16452 | NM_001048177.1 |
| | | | | | NM_008413.2 |
| EID1 | PNAS-22, C15orf3, | 23741 | NM_014335.2 | 58521 | NM_025613.3 |
| | CRI1, EID-1, IRO45620, | | | | |
| | PTD014, RBP21 | | | | |
| CFTR | tcag7.78, ABC35, | 1080 | NM_000492.3 | 12638 | NM_021050.2 |
| | ABCC7, CF, CFTR/MRP, | | | | |
| | MRP7, TNR-CFTR, | | | | |
| | dJ760C5.1 | | | | |
| TADA3 | ADA3, NGG1, STAF54, | 10474 | NM_001278270.1 | 101206 | NM_133932.2 |
| | TADA3L, Hada3 | | NR_103488.1 | | |
| | | | NM_006354.3 | | |
| | | | NM_133480.2 | | |
| MYBL2 | B-MYB, BMYB | 4605 | NM_002466.2 | 17865 | NM_008652.2 |
| KAT2A | GCN5, GCN5L2, PCAF- | 2648 | NM_021078.2 | 14534 | NM_001038010.2 |
| | b, Hgcn5 | | | | NM_020004.5 |
| IDH1 | IDCD, IDH, IDP, IDPC, | 3417 | NM_005896.2 | 15926 | NM_001111320.1 |
| | PICD | | | | NM_010497.3 |
| SUMO1 | OK/SW-cl.43, DAP1, | 7341 | NM_001005781.1 | 22218 | NM_009460.2 |
| | GMP1, OFC10, PIC1, | | NM_001005782.1 | | |
| | SENP2, SMT3, SMT3C, | | NM_003352.4 | | |
| | SMT3H3, UBL1 | | | | |
| SPEN | RP1-134O19.1, | 23013 | NM_015001.2 | 56381 | NM_019763.2 |
| | HIAA0929, MINT, | | | | |
| | RBM15C, SHARP | | | | |
| PRKDC | DNA-PKcs, DNAPK, | 5591 | NM_001081640.1 | 19090 | NM_011159.2 |
| | DNPK1, HYRC, HYRC1, | | NM_006904.6 | | |
| | XRCC7, p350 | | | | |
| KIR2DL4 | XXbac-BCX195L8.3, | 3805 | NM_001080770.1 | | |
| | CD158D, G9P, KIR103, | | NM_002255.5 | | |
| | KIR103AS | | | | |
| APC | BTPS2, DP2, DP2.5, | 324 | NM_000038.5 | 11789 | NM_007462.3 |
| | DP3, GS, PPP1R46 | | NM_001127510.2 | | |
| | | | NM_001127511.2 | | |
| MEF2D | RP11-98G7.2 | 4209 | NM_001271629.1 | 17261 | NM_133665.3 |
| | | | NM_005920.3 | | |
| MEAF6 | RP3-423B22.2, | 64769 | NM_001270875.1 | 70088 | NM_027310.3 |
| | C1orf149, CENP-28, | | NM_001270876.1 | | |
| | EAF6, NY-SAR-91 | | NM_022756.5 | | |
| | | | NR_073090.1 | | |
| | | | NR_073091.1 | | |
| | | | NR_073092.1 | | |
| TRRAP | PAF350/400, PAF400, | 8295 | NM_001244580.1 | 100683 | NM_001081362.1 |
| | STAF40, TR-AP, Tra1 | | NM_003496.3 | | |
| Eaf1 | EAF1 | 85403 | NM_033083.6 | 74427 | NM_028932.4 |
| EP400 | CAGH32, P400, | 57634 | NM_015409.4 | 75560 | NM_029337.2 |
| | TNRC12 | | | | NM_173066.1 |
| EPC1 | Epl1 | 80314 | NM_001272004.1 | 13831 | NM_001276350.1 |
| | | | NM_001272019.1 | | NM_007935.2 |
| | | | NM_025209.3 | | NM_027497.3 |
| DMAP1 | RP5-891H21.2, | 55929 | NM_001034023.1 | 66233 | NM_023178.2 |
| | DNMAP1, DNMTAP1, | | NM_019100.4 | | |
| | EAF2, MEAF2, SWC4 | | NM_001034024.1 | | |
| Tip60 | KAT5, ESA1, HTATIP, | 10524 | NM_001206833.1 | 81601 | NM_001199247.1 |
| | HTATIP1, PLIP, TIP, | | NM_006388.3 | | NM_001199248.1 |
| | ZC2HC5, Cpla2 | | NM_182709.2 | | NM_001199249.1 |
| | | | NM_182710.2 | | NM_178637.2 |
| | | | | | NR_037603.1 |
| MRG15 | MORF4L1, FWP006, | 10933 | NM_001265603.1 | 56397 | NM_001168225.1 |
| | Eaf3, HsT17725, | | NM_001265604.1 | | NM_001168226.1 |
| | MEAF3, MORFRG15, | | NM_001265605.1 | | NM_001168227.1 |
| | S863-6 | | NM_006791.3 | | NM_001168228.1 |
| | | | NM_206839.2 | | NM_001168229.1 |
| | | | | | NM_001168230.1 |
| | | | | | NM_019768.4 |
| MRGX | MORF4L2, MORFL2 | 9643 | NM_001142418.1 | 56397 | NM_001168225.1 |
| | | | NM_001142419.1 | | NM_001168226.1 |
| | | | NM_001142420.1 | | NM_001168227.1 |
| | | | NM_001142421.1 | | NM_001168228.1 |
| | | | NM_001142422.1 | | NM_001168229.1 |
| | | | NM_001142423.1 | | NM_001168230.1 |
| | | | NM_001142424.1 | | NM_019768.4 |
| | | | NM_001142425.1 | | |
| | | | NM_001142426.1 | | |
| | | | NM_001142427.1 | | |
| | | | NM_001142428.1 | | |
| | | | NM_001142429.1 | | |
| | | | NM_001142430.1 | | |
| | | | NM_001142431.1 | | |
| | | | NM_001142432.1 | | |
| | | | NM_012286.2 | | |
| MORF4 | CSR, CSRB, SEN, SEN1 | 10934 | No transcript avail | 67568 | NM_026242.3 |
| ACTB | BRWS1, PS1TP5BP1 | 60 | NM_001101.3 | 11461 | NM_007393.3 |
| ING1 | RP11-8D7.1, | 3621 | NM_001267728.1 | 26356 | NM_011919.4 |
| | p24ING1c, p33, | | NM_198219.2 | | |
| | P33ing1, p33ING1b, | | NM_005537.4 | | |
| | p47, p47ING1a | | NM_198217.2 | | |
| | | | NM_198218.2 | | |
| ING2 | ING1L, P33ing2 | 3622 | NM_001564.2 | 69260 | NM_023503.3 |
| ING3 | HSPC301, Eaf4, | 54556 | NM_019071.2 | 71777 | NM_023626.4 |
| | MEAF4, p471NG3, | | NM_198267.1 | | |
| ING4 | My036, my036, | 51147 | NM_001127582.1 | 28019 | NM_133345.2 |
| | p29ING4 | | NM_001127583.1 | | |
| | | | NM_001127584.1 | | |
| | | | NM_001127585.1 | | |
| | | | NM_001127586.1 | | |
| | | | NM_016162.3 | | |
| ING5 | p28ING5 | 84289 | NM_032329.4 | 66262 | NM_025454.2 |
| Eaf5 | Mortality factor | 10934, | NM_001265603.1 | 21761 | NM_001039147.2 |
| | related genes | 10933 | NM_001265604.1 | | NM_024431.3 |
| | (MORF4, MRG15/X) | | NM_001265605.1 | | |
| | | | NM_006791.3 | | |
| | | | NM_206839.2 | | |
| AF9 | MLLT3, YEATS3 | 4300 | NM_004529.2 | 70122 | NM_027326.3 |
| | | | | | NM_029931.2 |
| ENL | MLLT1, LTG19, YEATS1 | 4298 | NM_005934.3 | 64144 | NM_022328.2 |
| RUVBL1 | ECP54, INO80H, | 8607 | NM_003707.2 | 56505 | NM_019685.2 |
| | NMP238, PONTIN, | | | | |
| | Pontin52, RVB1, TIH1, | | | | |
| | TIP49, TIP49A | | | | |
| RUVBL2 | CGI-46, ECP51, | 10856 | NM_006666.1 | 20174 | NM_011304.3 |
| | INO80J, REPTIN, RVB2, | | | | |
| | TIH2, TIP48, TIP49B | | | | |
| SUV39H1 | MG44, KMT1A, | 6839 | NM_003173.2 | 20937 | NM_011514.2 |
| | SUV39H | | | | |
| SUV39H2 | RP11-2K17.2, KMT1B | 79723 | NM_001193424.1 | 64707 | NM_022724.4 |
| | | | NM_001193425.1 | | |
| | | | NM_001193426.1 | | |
| | | | NM_001193427.1 | | |
| | | | NM_024670.3 | | |
| SETDB1 | RP11-316M1.1, ESET, | 9869 | NM_001145415.1 | 84505 | NM_001163641.1 |
| | H3-K9-HMTase4, | | NM_001243491.1 | | NM_001163642.1 |
| | KG1T, KMT1E, TDRD21 | | NM_012432.3 | | NM_018877.3 |
| PRDM2 | RP5-1177E19.1, | 7799 | NM_001007257.2 | 110593 | NM_001081355.3 |
| | HUMHOXY1, KMT8, | | NM_001135610.1 | | NM_001256380.1 |
| | MTB-ZF, RIZ, RIZ1, | | NM_012231.4 | | |
| | RIZ2 | | NM_015866.4 | | |
| G9A | EHMT2, DAAP- | 10919 | NM_006709.3 | 110147 | NM_145830.1 |
| | 66K18.3, BAT8, | | NM_025256.5 | | NM_147151.1 |
| | C6orf30, G9A, GAT8, | | | | |
| | KMT1C, NG36 | | | | |
| EHMT1 | RP11-188C12.1, | 79813 | NM_001145527.1 | 77683 | NM_001012518.3 |
| | EUHMTASE1, EuHMTase1, | | NM_024757.4 | | NM_001109686.2 |
| | FP13812, | | | | NM_001109687.2 |
| | GLP, GLP1, KMT1D, | | | | NM_172545.4 |
| | bA188C12.1 | | | | |

In some embodiments, a epigenetic regulator of FXN may be a component of the NuA4 Histone Acetyltransferase Complex. The NuA4 histone acetyltransferase complex is a complex having histone acetylase activity on chromatin, as well as ATPase, DNA helicase and structural DNA binding activities. Subunits of the human complex include YEATS4, Eaf1, TRRAP, P400, EPC1, DMAP1, Tip60, MRG15, MRGX, MORF4, ACTB, ACTL6A, ING1, ING2, ING3, ING4, ING5, RUVBL1, RUVBL2, AF9, ENL, and MEAF6.

In some embodiments, a negative epigenetic regulator of FXN may be a histone-lysine N-methyltransferase. Histone-lysine N-methyltransferases catalyze the transfer of one, two or three methyl groups to a lysine residue of a histone protein. In some embodiments, the histone-lysine N-methyltransferase is capable of transferring one, two or three methyl groups to lysine 9 on histone H3 (H3K9me3). Methylation of lysine 9 on histone H3, especially near a gene promoter, is thought to reduce gene expression. H3K9me3 histone-lysine N-methyltransferases are well-known in the art and include SUV39H1, SUV39H2, SETDB1, PRDM2, G9A and EHMT1.

### Positive Epigenetic Regulators of FXN

As used herein, a “positive epigenetic regulator” is a regulatory factor (e.g., a regulatory protein) that inhibits the formation or maintenance of heterochromatin, and/or that promotes the formation or maintenance of euchromatin.

Without wishing to be bound by theory, it is believed that in some embodiments the heterochromatin formation at the FXN gene can be reversed, in part or in whole, by increasing the expression of one or more positive epigenetic regulators of FXN, thereby causing upregulation of FXN expression. Accordingly, in some embodiments, a positive epigenetic regulator of FXN induces expression of FXN by directly or indirectly inhibiting the formation or maintenance of heterochromatin at an FXN gene, and/or promoting the formation or maintenance of euchromatin at an FXN gene. Accordingly, in some embodiments, when the expression level of a positive epigenetic regulator of FXN is induced or increased, FXN expression may be upregulated.

In some embodiments, a positive epigenetic regulator of FXN is the product of a gene listed in Table 6 and/or 9 that has a fold change less than 1. In some embodiments, a positive epigenetic regulator of FXN is the product of a gene listed in Table 6 and/or 9 that has a fold change less than 0.75. In some embodiments, a positive epigenetic regulator of FXN is the product of a gene listed in Table 6 and/or 9 that has a fold change less than 0.5. In some embodiments, a positive epigenetic regulator of FXN is the product of a gene listed in Table 6 and/or 9 that has a fold change less than 0.25.

In some embodiments, a positive regulator of FXN is the product of a gene listed in Table 8.

### FXN Epigenetic Regulatory Pathways

As described in the Examples below, several pathways were identified that are enriched for epigenetic regulators of FXN. Thus, other components of these identified pathways are also contemplated as epigenetic regulators of FXN. Accordingly, in some embodiments, an epigenetic regulator of FXN is a component of the histone H2A acetylation pathway, the NuA4 histone acetyltransferase complex, the protein amino acid acetylation pathway, the histone acetylation pathway, the protein amino acid acylation pathway, the H4/H2A histone acetyltransferase complex, the nucleotide binding pathway, the histone H4 acetylation pathway, the histone acetyltransferase complex, or the insulin receptor substrate binding pathway.

Components of each pathway may be identified using the Gene ontology reference ID provided for each pathway in Table 7 (“GO:######”). The reference ID can be entered into the search function of the Gene Ontology website, and gene product associations can be identified. These gene product associations indicate other potential epigenetic regulators of FXN. In some embodiments, negative epigenetic regulators of FXN that are components of certain pathways are provided in Table 7. In some embodiments, positive epigenetic regulators of FXN that are components of certain pathways are provided in Table 8.

### Methods for Modulating FXN Gene Expression

In some aspects, the invention relates to methods for modulating FXN gene expression cells (e.g., cells for which FXN levels are reduced) for research purposes. In other aspects, the invention relates to methods for modulating gene expression in cells (e.g., cells for which FXN levels are reduced) for therapeutic purposes. Cells can be in vitro, ex vivo, or in vivo (e.g., in a subject who has a disease resulting from reduced expression or activity of FXN, e.g., Friedreich's ataxia.) In some embodiments, methods for modulating FXN expression in cells comprise delivering to the cells an oligonucleotide that inhibits expression or activity of a negative epigenetic regulator of FXN. In some embodiments, methods for modulating FXN expression in cells comprise delivering to the cells an inhibitor that inhibits activity of a negative epigenetic regulator of FXN. In some embodiments, methods for modulating FXN expression cells comprise delivering to the cells a cDNA engineered to express a positive epigenetic regulator of FXN.

It is understood that any reference to uses of compounds (e.g., oligonucleotides, expression vectors, inhibitors) throughout the description contemplates use of the compound in preparation of a pharmaceutical composition or medicament for use in the treatment of condition or a disease (e.g., Friedreich's ataxia) associated with decreased levels or activity of FXN. Thus, as one non-limiting example, this aspect of the invention includes use of oligonucleotides or inhibitors in the preparation of a medicament for use in the treatment of disease, wherein the treatment involves upregulating expression of FXN. In another non-limiting example, this aspect of the invention includes use of expression vector (e.g., containing a coding region of a positive epigenetic regulator of FXN) in the preparation of a medicament for use in the treatment of disease, wherein the treatment involves upregulating expression of FXN.

In some embodiments, methods provided herein comprise contacting a cell having a lower level of FXN expression compared to an appropriate control level of FXN expression with a composition (e.g., oligonucleotide, expression vector, inhibitor) useful for upregulating FXN expression.

In some embodiments, methods provided herein comprise contacting a cell having a lower level of FXN expression compared to an appropriate control level of FXN expression with an oligonucleotide specific for an mRNA of a negative epigenetic regulator of FXN as described herein, wherein the oligonucleotide reduces an expression level of the negative epigenetic regulator of FXN), thereby increasing FXN expression in the cell. In some embodiments, it is contemplated that the cell may be contacted with more than one oligonucleotide that targets one or more negative epigenetic regulators of FXN, e.g., a first oligonucleotide that targets a first negative epigenetic regulator of FXN as described herein and a second oligonucleotide that targets a second negative epigenetic regulator of FXN as described herein.

In another aspect of the invention, provided herein are methods for inhibiting the function of a negative epigenetic regulator of FXN (e.g., by contacting a cell with an appropriate inhibitor as described herein), thereby upregulating FXN expression. In some embodiments, provided are methods for increasing FXN expression in a cell by using one more inhibitors of histone-lysine N-methyltransferase. In some embodiments, the histone-lysine N-methyltransferase is capable of transferring one, two or three methyl groups to lysine 9 on histone H3 (H3K9me3). In some embodiments, the histone-lysine N-methyltransferase is SUV39H1. In some embodiments, the methods involve delivering to a cell an inhibitor that inhibits HLM, thereby increasing FXN expression in the cell. In some embodiments, a change in the chromatin state of the FXN gene (e.g., a decreased level of histone H3 K9 methylation at the FXN gene) increases expression of FXN. In some embodiments, the inhibitor is a small molecule inhibitor.

In certain embodiments, the level of expression of FXN using a histone-lysine N-methyltransferase inhibitor (HLMi) is increased by at least about 1.1×-1.5×, 1.5×-2×, 2×-2.5×, 2.5×-3×, or 3×-4× the control level of FXN expression.

In some embodiments, a cell having a lower level of FXN expression compared to an appropriate control level of FXN expression has a level of FXN expression that is at least 5%, 10%, 20%, 30%, 40%, 50%, 60%, 70%, 80%, 90%, 100%, 200%, 300%, 400%, 500% or more lower than an appropriate control level of FXN expression. A level of FXN expression may be determined using any suitable assay known in the art (see, e.g., Molecular Cloning: A Laboratory Manual, J. Sambrook, et al., eds., Third Edition, Cold Spring Harbor Laboratory Press, Cold Spring Harbor, N. Y., 2001; Current Protocols in Molecular Biology, Current Edition, John Wiley & Sons, Inc., New York; and Current Protocols in Protein Production, Purification, and Analysis, Current Edition, John Wiley & Sons, Inc., New York). The FXN expression level may be an mRNA level or a protein level. The sequences of FXN mRNAs and proteins are well-known in the art (see, e.g., NCBI Transcript IDs: NM_000144.4, NM_001161706.1, and NM_181425.2, and NCBI Protein IDs: NP_000135.2, NP_001155178.1, and NP_852090.1) and can be used to design suitable reagents and assays for measuring an FXN expression level.

In some embodiments, an appropriate control level of FXN expression may be, e.g., a level of FXN expression in a cell, tissue or fluid obtained from a healthy subject or population of healthy subjects. As used herein, a healthy subject is a subject that is apparently free of disease and has no history of disease, e.g., no history of Friedreich's ataxia. In some embodiments, an appropriate control level of is a level of FXN expression in a cell from a subject that does not have Friedreich's ataxia or a level of FXN expression in a population of cells from a population of subjects that do not have Friedreich's ataxia. In some embodiments, the subject or population of subjects that do not have Friedreich's ataxia are subjects that have a FXN gene locus that contains less than 20, 19, 18, 17, 16, 15, 14, 13, 12, 11, or 10 GAA repeat units in the first intron. In some embodiments, when a level of FXN expression is elevated or increased compared to a control level of FXN, an appropriate control level of FXN may be a level of FXN expression in a cell, tissue, or subject to which an oligonucleotide has not been delivered or to which a negative control has been delivered (e.g., a scrambled oligo, a carrier, etc.).

In some embodiments, an appropriate control level of FXN expression may be a predetermined level or value, such that a control level need not be measured every time. The predetermined level or value can take a variety of forms. It can be single cut-off value, such as a median or mean. It can be established based upon comparative groups, such as where one defined group is known have Friedriech's ataxia and another defined group is known to not have Friedriech's ataxia. It can be a range, for example, where the tested population is divided equally (or unequally) into groups, such as a group of subjects having a high number of GAA repeats in the first intron of FXN (e.g., over 1000 GAA repeats), a group of subjects having a moderate number of GAA repeats (e.g., from 20-1000 GAA repeats) and a group of subjects having a low number of GAA repeats (e.g., less than 20 GAA repeats).

The predetermined value can depend upon the particular population selected. Accordingly, the predetermined values selected may take into account the category in which a subject falls. Appropriate ranges and categories can be selected with no more than routine experimentation by those of ordinary skill in the art.

In some embodiments, a cell having a lower level of FXN expression compared to an appropriate control level of FXN expression is a cell that has a higher level of histone H3 K27 or K9 methylation at the FXN gene compared with an appropriate control level of histone H3 K27 or K9 methylation. An appropriate control level of histone H3 K27 or K9 methylation may be, e.g., a level of histone H3 K27 or K9 methylation in a cell, tissue or fluid obtained from a healthy subject or population of healthy subjects, such as a subject or subjects that do not have Friedreich's ataxia. A level of H3 K27 or K9 methylation expression may be determined using any suitable assay known in the art. Examples of assays for detecting histone methylation levels include, but are not limited to, immunoassays such as Western blot, immunohistochemistry and ELISA assays. Such assays may involve a binding partner, such as an antibody, that specifically binds to a methylated or unmethylated histone. Antibodies that recognize specific methylation patterns on histones are known in the art and available from commercial vendors (see, e.g., AbCam and Millipore).

In some embodiments, a cell having a lower level of FXN expression compared to an appropriate control level of FXN expression is a cell that comprises an FXN gene encoding in its first intron a GAA repeat of between 10-2000, 15-2000, 20-2000, 30-2000, 40-2000, 50-2000, 100-2000, 10-1000, 15-1000, 20-1000, 30-1000, 40-1000, 50-1000, or 100-1000 units. The number of GAA repeats may be determined using any method known in the art, e.g., sequencing-based assays or probe-based assays.

In some embodiments, a cell having a lower level of FXN expression compared to an appropriate control level of FXN expression is a cell obtained from a subject having Friedreich's ataxia. A subject having Friedreich's ataxia can be identified, e.g., by the number of GAA repeats present in the first intron of an FXN gene of the subject and/or by other diagnostic criteria or symptoms known in the art. Symptoms of Friedreich's ataxia include, but are not limited to, muscle weakness in the arms and legs, loss of coordination, vision impairment, hearing impairment, slurred speech, curvature of the spine, high plantar arches, diabetes, and/or heart disorders (e.g., cardiomegaly, atrial fibrillation, tachycardia and hypertrophic cardiomyopathy). A physical examination of eye movements, deep tendon reflexes, extensor plantar responses, and cardiac sounds may aid in diagnosis of a subject suspected of having Friedreich's ataxia. A genetic test, e.g., a PCR-based test, may be used to identify a subject having expanded GAA triplet repeats in the first intron of FXN.

As used herein, reducing an expression level of a negative epigenetic regulator of FXN includes reducing an expression level of the negative epigenetic regulator of FXN to 1%, 5%, 10%, 20%, 30%, 40%, 50%, 60%, 70%, 80%, 90%, 100%, 150%, 200%, 300%, 400%, 500% or more lower than an appropriate control level. An appropriate control level may be, e.g., a level of the negative epigenetic regulator of FXN in a cell that has not been contacted with an oligonucleotide or inhibitor as described herein. The expression level of the negative epigenetic regulator of FXN may be an mRNA level or a protein level. Thus, an oligonucleotide as described herein may reduce the mRNA and/or protein level of the negative epigenetic regulator of FXN. For example, if the oligonucleotide is designed to degrade the mRNA, the level of mRNA will be reduced, and subsequently the level of protein will also be reduced. In another example, if the oligonucleotide is designed to block translation, the level of protein will be reduced, but the level of mRNA may remain stable. Assays for determining mRNA and protein levels are well-known in the art (e.g., microarrays, sequencing-based assays, probe-based assays, immunoassays, mass-spectrometry, etc.).

As used herein, increasing FXN expression in a cell includes a level of FXN expression that is, e.g., 1%, 5%, 10%, 20%, 30%, 40%, 50%, 60%, 70%, 80%, 90%, 100%, 150%, 200%, 300%, 400%, 500% or more above an appropriate control level of FXN. The appropriate control level may be a level of FXN expression in a cell that has not been contacted with an oligonucleotide or inhibitor as described herein. The FXN expression may be FXN mRNA and/or protein expression. In some embodiments, increasing FXN expression in a cell includes increasing a level of FXN expression to within 50%, 40%, 30%, 20%, 10%, 5%, 4%, 3%, 2%, 1%, or less of a level of FXN expression in a cell from a healthy subject or a population of cells from a population of healthy subjects, e.g., subjects that do not have Friedreich's ataxia. For example, it may be desirable to increase an FXN expression level in a cell obtained from or in subject having Friedreich's ataxia such that the level of FXN expression is approximately the same as the level of FXN expression in a cell obtained from or in a subject who is healthy (e.g., not having Friedreich's ataxia). However, it is to be understood that the level of FXN expression level in a cell obtained from or in subject having Friedreich's ataxia may be increased to a level that is higher than the level of FXN expression in a cell obtained from or in a subject who is healthy.

In another aspect of the invention, methods comprise administering to a subject (e.g. a human) a composition as described herein (e.g., a composition comprising an oligonucleotide and/or inhibitor targeting a negative epigenetic regulator of FXN) to increase FXN protein levels in the subject. In some embodiments, the increase in protein levels is at least 5%, 10%, 20%, 30%, 40%, 50%, 60%, 70%, 80%, 90%, 100%, 200%, or more, higher than the amount of a protein in the subject before administering the oligonucleotide and/or inhibitor.

Aspects of the invention relate to compositions and methods of treating a condition (e.g., Friedreich's ataxia) associated with decreased levels of expression of FXN in a subject. An appropriate subject may be a non-human mammal, e.g. mouse, rat, guinea pig, rabbit, cat, dog, goat, cow, or horse. In preferred embodiments, a subject is a human. Oligonucleotides and inhibitors have been employed as therapeutic moieties in the treatment of disease states in animals, including humans. Oligonucleotides and inhibitors can be useful therapeutic modalities that can be configured to be useful in treatment regimes for the treatment of cells, tissues and animals, especially humans.

### Oligonucleotides for Modulating Expression of FXN

In one aspect of the invention, oligonucleotides are provided for modulating expression of FXN in a cell. In some embodiments, expression of FXN is upregulated or increased. In some embodiments, oligonucleotides are provided that reduce the expression level of a negative epigenetic regulator of FXN, thereby upregulating the expression of FXN. In some embodiments, the oligonucleotide is specific for an mRNA of a negative epigenetic regulator of FXN.

The oligonucleotide may be single stranded or double stranded. Single stranded oligonucleotides may include secondary structures, e.g., a loop or helix structure. In some embodiments, the oligonucleotide comprises at least one modified nucleotide or modified internucleoside linkage as described herein.

The oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches.

The oligonucleotide may have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than a negative epigenetic regulator of FXN. The threshold level of sequence identity may be 50%, 60%, 70%, 80%, 85%, 90%, 95%, 99% or 100% sequence identity.

The oligonucleotide may have a sequence that is has greater than 30% G-C content, greater than 40% G-C content, greater than 50% G-C content, greater than 60% G-C content, greater than 70% G-C content, or greater than 80% G-C content. The oligonucleotide may have a sequence that has up to 100% G-C content, up to 95% G-C content, up to 90% G-C content, or up to 80% G-C content. In some embodiments in which the oligonucleotide is 8 to nucleotides in length, all but 1, 2, 3, 4, or 5 of the nucleotides of the complementary sequence of the mRNA of a negative epigenetic regulator of FXN are cytosine or guanosine nucleotides. In some embodiments, the sequence of the mRNA to which the oligonucleotide is complementary comprises no more than 3 nucleotides selected from adenine and uracil.

The oligonucleotide may be complementary to a chromosome of a different species (e.g., a mouse, rat, rabbit, goat, monkey, etc.) at a position that encompasses or that is in proximity to that species' homolog of the negative epigenetic regulator of FXN. The oligonucleotide may be complementary to a human genomic region encompassing or in proximity to the negative epigenetic regulator of FXN and also be complementary to a mouse genomic region encompassing or in proximity to the mouse homolog of the negative epigenetic regulator of FXN. For example, the oligonucleotide may be complementary to a sequence of a human mRNA of a negative epigenetic regulator of FXN (for example, a human mRNA referenced in Table 1 by its NCBI accession number), and also be complementary to a sequence of the corresponding mouse mRNA of the negative epigenetic regulator of FXN (for example, a corresponding mouse mRNA referenced in Table 1 by its NCBI accession number). Oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of the oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of an mRNA of a negative epigenetic regulator of FXN. In some embodiments, the region of complementarity is complementary with at least 8 consecutive nucleotides of an mRNA of a negative epigenetic regulator of FXN. In some embodiments the sequence of the oligonucleotide is based on an RNA sequence that binds to an mRNA of a negative epigenetic regulator of FXN, or a portion thereof, said portion having a length of from 5 to 40 contiguous base pairs, or about 8 to 40 bases, or about 5 to 15, or about 5 to 30, or about 5 to 40 bases, or about 5 to 50 bases.

Complementary, as the term is used in the art, refers to the capacity for precise pairing between two nucleotides. For example, if a nucleotide at a certain position of an oligonucleotide is capable of hydrogen bonding with a nucleotide at the same position of an mRNA of a negative epigenetic regulator of FXN, then the oligonucleotide and the mRNA of a negative epigenetic regulator of FXN are considered to be complementary to each other at that position. The oligonucleotide and the mRNA of a negative epigenetic regulator of FXN are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and the mRNA of a negative epigenetic regulator of FXN. For example, if a base at one position of an oligonucleotide is capable of hydrogen bonding with a base at the corresponding position of an mRNA of a negative epigenetic regulator of FXN, then the bases are considered to be complementary to each other at that position. 100% complementarity is not required.

The oligonucleotide may be at least 80% complementary to (optionally one of at least 85%, 90%, 91%, 92%, 93%, 94%, 95%, 96%, 97%, 98%, 99% or 100% complementary to) the consecutive nucleotides of an mRNA of a negative epigenetic regulator of FXN. In some embodiments the oligonucleotide may contain 1, 2 or 3 base mismatches compared to the portion of the consecutive nucleotides of an mRNA of a negative epigenetic regulator of FXN. In some embodiments the oligonucleotide may have up to 3 mismatches over 15 bases, or up to 2 mismatches over 10 bases.

It is understood in the art that a complementary nucleotide sequence need not be 100% complementary to that of its target to be specifically hybridizable or specific for a target molecule. In some embodiments, a complementary nucleic acid sequence for purposes of the present disclosure is specifically hybridizable or specific for the target molecule when binding of the sequence to the target molecule (e.g., mRNA) interferes with the normal function of the target (e.g., mRNA) to cause a loss of activity (e.g., inhibiting translation with consequent up-regulation of FXN gene expression) or expression (e.g., degrading the mRNA with consequent up-regulation of FXN gene expression) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. In a preferred embodiment, the oligonucleotide is 8 to 30 nucleotides in length.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, GC content of the oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

It is to be understood that any oligonucleotide provided herein can be excluded.

In some embodiments, it has been found that oligonucleotides disclosed herein may increase expression of FXN mRNA by at least about 50% (i.e. 150% of normal or 1.5 fold), or by about 2 fold to about 5 fold. In some embodiments, expression may be increased by at least about 15 fold, 20 fold, 30 fold, 40 fold, 50 fold or 100 fold, or any range between any of the foregoing numbers.

Any suitable oligonucleotide for targeting an mRNA is contemplated here. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA.

### Oligonucleotide Structure and Modifications

The oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not mediate alternative splicing; are not immune stimulatory; are nuclease resistant; have improved cell uptake compared to unmodified oligonucleotides; are not toxic to cells or mammals; or have improved endosomal exit.

Oligonucleotides that are designed to interact with RNA to modulate gene expression are a distinct subset of base sequences from those that are designed to bind a DNA target (e.g., are complementary to the underlying genomic DNA sequence from which the RNA is transcribed).

Any of the oligonucleotides disclosed herein may be linked to one or more other oligonucleotides disclosed herein by a linker, e.g., a cleavable linker.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleoside linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

Any of the modified chemistries or formats of oligonucleotides described herein can be combined with each other, and that one, two, three, four, five, or more different types of modifications can be included within the same molecule.

In some embodiments, an oligonucleotide may comprise one or more modified nucleotides (also referred to herein as nucleotide analogs). In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States patent or patent application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the oligonucleotide has one or more nucleotide analogues. For example, the oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Preferably the oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

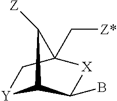

where X and Y are independently selected among the groups —O—,

—S—, —N(H)—, N(R)—, —CH₂— or —C— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

In some embodiments, the LNA used in the oligonucleotides described herein comprises at least one LNA unit according any of the formulas

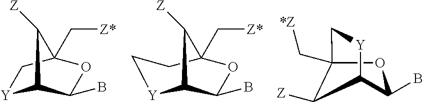

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from —O—P(O)₂—O—, —O—P(O,S)—O—, -0-P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, -0-PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown below:

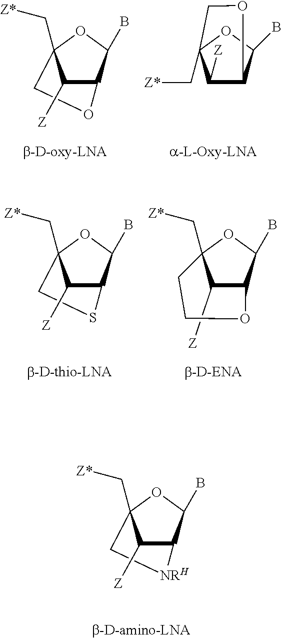

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)—where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, oligonucleotide modification includes modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of the oligonucleotide. In some embodiments, the oligonucleotide comprises a biotin moiety conjugated to the 5′ nucleotide.

In some embodiments, the oligonucleotide comprises locked nucleic acids (LNA), ENA modified nucleotides, 2′-O-methyl nucleotides, or 2′-fluoro-deoxyribonucleotides. In some embodiments, the oligonucleotide comprises alternating deoxyribonucleotides and 2′-fluoro-deoxyribonucleotides. In some embodiments, the oligonucleotide comprises alternating deoxyribonucleotides and 2′-O-methyl nucleotides. In some embodiments, the oligonucleotide comprises alternating deoxyribonucleotides and ENA modified nucleotides. In some embodiments, the oligonucleotide comprises alternating deoxyribonucleotides and locked nucleic acid nucleotides. In some embodiments, the oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides.

It should be appreciated that the oligonucleotide can have any combination of modifications as described herein.

In some embodiments, an oligonucleotide described herein may be a mixmer or comprise a mixmer sequence pattern. The term ‘mixmer’ refers to oligonucleotides which comprise both naturally and non-naturally occurring nucleotides or comprise two different types of non-naturally occurring nucleotides. Mixmers are generally known in the art to have a higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule.

In some embodiments, the mixmer comprises or consists of a repeating pattern of nucleotide analogues and naturally occurring nucleotides, or one type of nucleotide analogue and a second type of nucleotide analogue. However, it is to be understood that the mixmer need not comprise a repeating pattern and may instead comprise any arrangement of nucleotide analogues and naturally occurring nucleotides or any arrangement of one type of nucleotide analogue and a second type of nucleotide analogue. The repeating pattern, may, for instance be every second or every third nucleotide is a nucleotide analogue, such as LNA, and the remaining nucleotides are naturally occurring nucleotides, such as DNA, or are a 2′ substituted nucleotide analogue such as 2′MOE or 2′ fluoro analogues, or any other nucleotide analogues described herein. It is recognized that the repeating pattern of nucleotide analogues, such as LNA units, may be combined with nucleotide analogues at fixed positions—e.g. at the 5′ or 3′ termini.

In some embodiments, the mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive nucleotide analogues, such as at least two consecutive LNAs. In some embodiments, the mixmer comprises at least a region consisting of at least three consecutive nucleotide analogue units, such as at least three consecutive LNAs.

In some embodiments, the mixmer does not comprise a region of more than 7, more than 6, more than 5, more than 4, more than 3, or more than 2 consecutive nucleotide analogues, such as LNAs. It is to be understood that the LNA units may be replaced with other nucleotide analogues, such as those referred to herein.

In some embodiments, the mixmer comprises at least one nucleotide analogue in one or more of six consecutive nucleotides. The substitution pattern for the nucleotides may be selected from the group consisting of Xxxxxx, xXxxxx, xxXxxx, xxxXxx, xxxxXx and xxxxxX, wherein “X” denotes a nucleotide analogue, such as an LNA, and “x” denotes a naturally occurring nucleotide, such as DNA or RNA.

In some embodiments, the mixmer comprises at least two nucleotide analogues in one or more of six consecutive nucleotides. The substitution pattern for the nucleotides may be selected from the group consisting of XXxxxx, XxXxxx, XxxXxx, XxxxXx, XxxxxX, xXXxxx, xXxXxx, xXxxXx, xXxxxX, xxXXxx, xxXxXx, xxXxxX, xxxXXx, xxxXxX and xxxxXX, wherein “X” denotes a nucleotide analogue, such as an LNA, and “x” denotes a naturally occurring nucleotide, such as DNA or RNA. In some embodiments, the substitution pattern for the nucleotides may be selected from the group consisting of XxXxxx, XxxXxx, XxxxXx, XxxxxX, xXxXxx, xXxxXx, xXxxxX, xxXxXx, xxXxxX and xxxXxX. In some embodiments, the substitution pattern is selected from the group consisting of xXxXxx, xXxxXx, xXxxxX, xxXxXx, xxXxxX and xxxXxX. In some embodiments, the substitution pattern is selected from the group consisting of xXxXxx, xXxxXx and xxXxXx. In some embodiments, the substitution pattern for the nucleotides is xXxXxx.

In some embodiments, the mixmer comprises at least three nucleotide analogues in one or more of six consecutive nucleotides. The substitution pattern for the nucleotides may be selected from the group consisting of XXXxxx, xXXXxx, xxXXXx, xxxXXX, XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx, wherein “X” denotes a nucleotide analogue, such as an LNA, and “x” denotes a naturally occuring nucleotide, such as DNA or RNA. In some embodiments, the substitution pattern for the nucleotides is selected from the group consisting of XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx. In some embodiments, the substitution pattern for the nucleotides is selected from the group consisting of xXXxXx, xXXxxX, xxXXxX, xXxXXx, xXxxXX, xxXxXX and xXxXxX. n some embodiments, the substitution pattern for the nucleotides is xXxXxX or XxXxXx. In some embodiments, the substitution pattern for the nucleotides is xXxXxX.

In some embodiments, the mixmer comprises at least four nucleotide analogues in one or more of six consecutive nucleotides. The substitution pattern for the nucleotides may be selected from the group consisting of xXXXX, xXxXXX, xXXxXX, xXXXxX, xXXXXx, XxxXXX, XxXxXX, XxXXxX, XxXXXx, XXxxXX, XXxXxX, XXxXXx, XXXxxX, XXXxXx and XXXXxx, wherein “X” denotes a nucleotide analogue, such as an LNA, and “x” denotes a naturally occurring nucleotide, such as DNA or RNA.

In some embodiments, the mixmer comprises at least five nucleotide analogues in one or more of six consecutive nucleotides. The substitution pattern for the nucleotides may be selected from the group consisting of xXXXXX, XxXXXX, XXxXXX, XXXxXX, XXXXxX and XXXXXx, wherein “X” denotes a nucleotide analogue, such as an LNA, and “x” denotes a naturally occuring nucleotide, such as DNA or RNA.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the mixmer contains a modified nucleotide, e.g., an LNA, at the 5′ end. In some embodiments, the mixmer contains a modified nucleotide, e.g., an LNA, at the first two positions, counting from the 5′ end.

In some embodiments, the mixmer is incapable of recruiting RNAseH.

Oligonucleotides that are incapable of recruiting RNAseH are well known in the literature, in example see WO2007/112754, WO2007/112753, or PCT/DK2008/000344. Mixmers may be designed to comprise a mixture of affinity enhancing nucleotide analogues, such as in non-limiting example LNA nucleotides and 2′-O-methyl nucleotides. In some embodiments, the mixmer comprises modified internucleoside linkages (e.g., phosphorothioate internucleoside linkages or other linkages) between at least two, at least three, at least four, at least five or more nucleotides.

A mixmer may be produced using any method known in the art or described herein. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, the oligonucleotide is a gapmer. A gapmer oligonucleotide generally has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAseH. Without wishing to be bound by theory, it is thought that the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., 1-6 modified nucleotides. Exemplary modified oligonucleotides include, but are not limited to, 2′ MOE or 2′OMe or Locked Nucleic Acid bases (LNA). The flanks X and Z may be have a of length 1-20 nucleotides, preferably 1-8 nucleotides and even more preferred 1-5 nucleotides. The flanks X and Z may be of similar length or of dissimilar lengths. The gap-segment Y may be a nucleotide sequence of length 5-20 nucleotides, preferably 6-12 nucleotides and even more preferred 6-10 nucleotides. In some aspects, the gap region of the gapmer oligonucleotides of the invention may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using any method known in the art or described herein. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective.

Following selection of an appropriate target RNA sequence, siRNA molecules that comprise a nucleotide sequence complementary to all or a portion of the target sequence, i.e. an antisense sequence, can be designed and prepared using any method known in the art (see, e.g., PCT Publication Nos. WO08124927A1 and WO 2004/016735; and U.S. Patent Publication Nos. 2004/0077574 and 2008/0081791). A number of commercial packages and services are available that are suitable for use for the preparation of siRNA molecules. These include the in vitro transcription kits available from Ambion (Austin, Tex.) and New England Biolabs (Beverly, Mass.) as described above; viral siRNA construction kits commercially available from Invitrogen (Carlsbad, Calif.) and Ambion (Austin, Tex.), and custom siRNA construction services provided by Ambion (Austin, Tex.), Qiagen (Valencia, Calif.), Dharmacon (Lafayette, Colo.) and Sequitur, Inc (Natick, Mass.). A target sequence can be selected (and a siRNA sequence designed) using computer software available commercially (e.g. OligoEngine™ (Seattle, Wash.); Dharmacon, Inc. (Lafayette, Colo.); Target Finder from Ambion Inc. (Austin, Tex.) and the siRNA Design Tool from QIAGEN, Inc. (Valencia, Calif.)). In some embodiments, an siRNA may be designed or obtained using the RNAi atlas (available at the RNAiAtlas website), the siRNA database (available at the Stockholm Bioinformatics Website), or using DesiRM (available at the Institute of Microbial Technology website).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 200 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 200 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present invention comprises 3′ overhangs of about 1 to about 3 nucleotides on both ends of the molecule. In some embodiments, an oligonucleotide may be a microRNA (miRNA).

MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides, although miRNAs of up to 2000 nucleotides can be utilized. In one embodiment the size range of the miRNA is from 70 to 170 nucleotides in length. In another embodiment, mature miRNAs of from 21 to 25 nucleotides in length can be used.

In some embodiments, a miRNA is expressed from a vector. In some embodiments, the vector may include a sequence encoding a mature miRNA. In some embodiments, the vector may include a sequence encoding a pre-miRNA such that the pre-miRNA is expressed and processed in a cell into a mature miRNA. In some embodiments, the vector may include a sequence encoding a pri-miRNA. In this embodiment, the primary transcript is first processed to produce the stem-loop precursor miRNA molecule. The stem-loop precursor is then processed to produce the mature microRNA.

In some embodiments, oligonucleotides provided herein may be in the form of aptamers._An “aptamer” is any nucleic acid that binds specifically to a target, such as a small molecule, protein, nucleic acid, cell, tissue or organism. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof.

Selection of nucleic acid aptamers may be accomplished by any suitable method known in the art, including an optimized protocol for in vitro selection, known as SELEX (Systemic Evolution of Ligands by Exponential enrichment). Many factors are important for successful aptamer selection. For example, the target molecule should be stable and easily reproduced for each round of SELEX, because the SELEX process involves multiple rounds of binding, selection, and amplification to enrich the nucleic acid molecules. In addition, the nucleic acids that exhibit specific binding to the target molecule have to be present in the initial library. Thus, it is advantageous to produce a highly diverse nucleic acid pool. Because the starting library is not guaranteed to contain aptamers to the target molecule, the SELEX process for a single target may need to be repeated with different starting libraries. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′, 5′-phosphate diester to a 2′, 3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

### Expression Vectors

It is to be appreciated that use of expression vectors to deliver oligonucleotides or any other appropriate nucleic acid (e.g., a cDNA engineered to expression a positive epigenetic regulator of FXN) is contemplated in any appropriate context. Vectors include, but are not limited to, plasmids, viral vectors, other vehicles derived from viral or bacterial or other sources that have been manipulated by the insertion or incorporation of the nucleic acid sequences for expressing an RNA transcript (e.g., shRNA, miRNA, mRNA).

In some embodiments, expression vectors are provided that are engineered to express a positive epigenetic regulator (e.g., a product of a gene as provided in Table 7). In some embodiments, expression of the positive epigenetic regulator causes upregulation of FXN. In some embodiments, an expression vector may be engineered by incorporating a cDNA comprising exons of a gene of interest into a plasmid that is suitably configured with expression elements (e.g., a promoter) for expressing the gene of interest. In some embodiments, cDNA may be obtained or synthesized using a commercially available kit or any method known in the art, e.g, synthesized from mature (fully spliced) mRNA using the enzyme reverse transcriptase (see, e.g., U.S. Pat. Nos. 7,470,515 and 8,420,324, and PCT Publication Numbers WO2000052191, WO1997024455).

In some embodiments, a vector may comprise one or more expression elements. “Expression elements” are any regulatory nucleotide sequence, such as a promoter sequence or promoter-enhancer combination, which facilitates the efficient expression of an RNA transcript (e.g., shRNA, miRNA, mRNA). The expression element may, for example, be a mammalian or viral promoter, such as a constitutive or inducible promoter or a tissue specific promoter, examples of which are well known to one of ordinary skill in the art. Constitutive mammalian promoters include polymerase promoters as well as the promoters for the following non-limiting genes: hypoxanthine phosphoribosyl transferase (HPTR), adenosine deaminase, pyruvate kinase, and beta-actin. Exemplary viral promoters which function constitutively in eukaryotic cells include promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters may be used. Inducible promoters are expressed in the presence of an inducing agent and include metal-inducible promoters and steroid-regulated promoters, for example. Other inducible promoters may be used.

Expression vectors may also comprise an origin of replication, a suitable promoter polyadenylation site, transcriptional termination sequences, and 5′ flanking nontranscribed sequences. DNA sequences derived from the SV40 viral genome, for example, SV40 origin, early promoter, enhancer, splice, and polyadenylation sites may be used to provide the required non-transcribed genetic elements.

One of skill in the art can readily employ other vectors known in the art. Viral vectors are generally based on non-cytopathic eukaryotic viruses in which non-essential genes have been replaced with the nucleic acid sequence of interest. Standard protocols for producing replication-deficient retroviruses (including the steps of incorporation of exogenous genetic material into a plasmid, transfection of a packaging cell lines with plasmid, production of recombinant retroviruses by the packaging cell lie, collection of viral particles from tissue culture media, and infection of the target cells with viral particles) may be used. Viral and retroviral vectors that may be used include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus.

### Formulation, Delivery, and Dosing

The compositions (e.g., oligonucleotides, expression vectors, inhibitors) described herein can be formulated for administration to a subject for treating a condition (e.g., Friedrich's ataxia) associated with decreased levels of FXN. The formulations may conveniently be presented in unit dosage form and may be prepared by any methods well known in the art of pharmacy. The amount of active ingredient (e.g., an oligonucleotide, expression vector, inhibitor) which can be combined with a carrier material to produce a single dosage form will vary depending upon the host being treated, the particular mode of administration, e.g., intrathecal, intraneural, intracerebral, intramuscular, etc. The amount of active ingredient which can be combined with a carrier material to produce a single dosage form will generally be that amount of the compound which produces a therapeutic effect.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated composition can assume a variety of states. In some examples, the composition is at least partially crystalline, uniformly crystalline, and/or anhydrous (e.g., less than 80, 50, 30, 20, or 10% water). In another example, the composition is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated or administered (together or separately) in combination with another agent, e.g., another therapeutic agent or an agent that stabilizes an oligonucleotide, e.g., a protein that complexes with the oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, an oligonucleotide preparation includes another oligonucleotide, e.g., a second oligonucleotide that modulates expression of a second gene or a second oligonucleotide that modulates expression of the first gene. Still other preparation can include at least 3, 5, 10, twenty, fifty, or a hundred or more different oligonucleotide species. Such oligonucleotides can mediated gene expression with respect to a similar number of different genes. In one embodiment, the oligonucleotide preparation includes at least a second therapeutic agent (e.g., an agent other than an oligonucleotide). Expression vectors expressing different positive epigenetic regulators may be similarly combined with one another. Expression vectors expressing different positive epigenetic regulators may also be combined with one or more oligonucleotides that target negative epigenetic regulators.

In some embodiments, one or more oligonucleotides as provided herein is combined with the use of one or more inhibitors as described herein.

### Histone-Lysine N-Methyltransferase Inhibitors for Modulating Expression of FXN

Provided herein are methods of increasing FXN expression (protein and/or mRNA) in a subject or cell using an inhibitor of a negative epigenetic regulator of FXN. IN some embodiments, a histone-lysine N-methyltransferase inhibitor (HLMi) is used. The HLMi are contacted with cells of interest, thereby inhibiting histone-lysine N-methyltransferase, decreasing the levels of histone H3 K9 methylation, and increasing FXN expression in the cell, wherein, prior to contact with the inhibitor, the cell has a lower level of FXN expression compared to an appropriate control level of FXN expression. The cell is obtained from or present in a subject having Friedreich's ataxia.

In certain embodiments, the inhibitor is from the epipolythiodioxopiperazine class of fungal metabolites. In certain embodiments, the inhibitor is chaetocin.

In certain embodiments, the inhibitor comprises a quinazoline scaffold. In certain embodiments, the inhibitor comprises a 2,4-diamino-6,7-dimethoxyquinazoline scaffold. In certain embodiments, the inhibitor is a compound with the following formula:

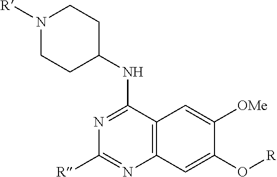

or pharmaceutically acceptable salts or solvates thereof. R is

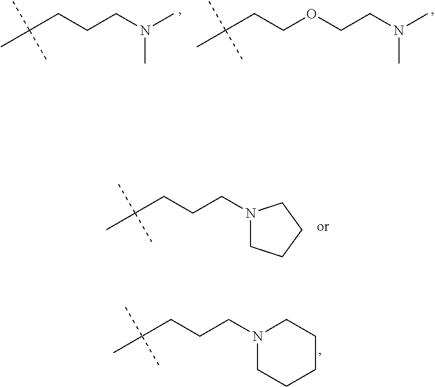

R′ is isopropyl, cyclohexyl, or benzyl. R″ is

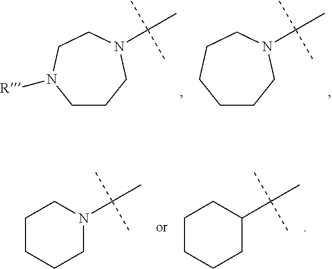

R′″ is methyl, ethyl, isopropyl, benzyl, cyclohexyl, or cyclohexylmethyl. In certain embodiments, the inhibitor is BIX01294, UNC0224, UNC0321, UNC0638, UNC0646, UNC0631, TM2-115, UNC0642, BIX-01338, or E72.

In certain embodiments, the inhibitor comprises an indole scaffold. In certain embodiments, the inhibitor is A-366.

In certain embodiments, the inhibitor comprises a benzimidazole scaffold. In certain embodiments, the benzimidazole scaffold is a 2-substituted benzimidazole. In certain embodiments, the benzimidazole scaffold is the following:

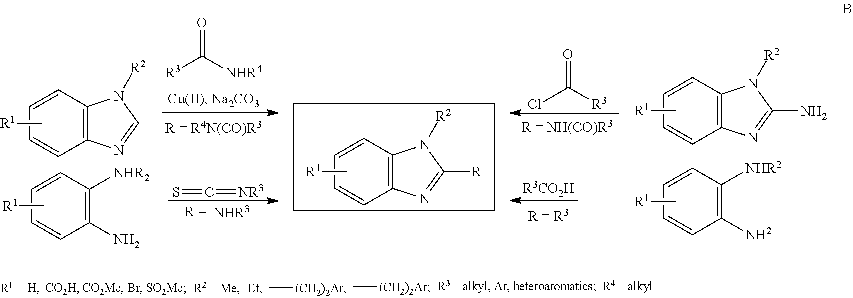

In certain embodiments, the inhibitor is BRD4770.

In certain embodiments, the inhibitor comprises an adenosine scaffold. In certain embodiments, the inhibitor comprising an adenosine scaffold is sinefungin or analogues thereof. In certain embodiments, the alpha-amino acid moiety in the sinefungin analogue has been exchanged to a moiety without an amino group. In certain embodiments, the inhibitor is 5′-desoxy-5′-butyladenosine. In certain embodiments, the alpha-amino acid moiety in the sinefungin analogue has been exchanged to a moiety with an amino group. In certain embodiments, the inhibitor is 5′-desoxy-5′-(2″-cyclohexyl-1″aminoethyl)-adenosine.

In some embodiments, one or more inhibitors of HML can be used to increase FXN expression. In certain embodiments, the inhibitor is one of the exemplary inhibitors listed in Table 2 or a pharmaceutically acceptable salt or solvate thereof. In certain embodiments, the inhibitor includes both the neutral form and a pharmaceutically acceptable salt thereof.

TABLE 2
Histone-lysine N-methyltransferase Inhibitors

| Agent | Structure |
|---|---|
| Chaetocin; (3S,3′S,5aR,5aR,10bR,10′bR,11aS,11′aS)- 2,2′,3,3′,5a,5′a,6,6′-octahydro- 3,3′-bis(hydroxymethyl)-2,2′- dimethyl-[10b,10′b(11H,11′H)- bi3,11a-epidithio-11aH- pyrazino[1′,2′:1,5]pyrrolo[2,3- b]indole]-1,1′4,4′-tetrone | 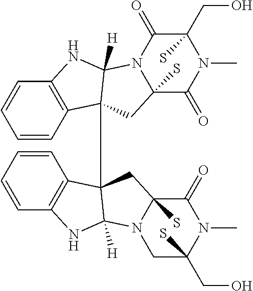 |
| BIX01294; 2-(Hexahydro-4-methyl- 1H-1,4-diazepin-1-yl)-6,7- dimethoxy-N-[1-(phenylmethyl)-4- piperidinyl]-4-quinazolinamine, (including other solvate forms such as the trihydrochloride hydrate form) | 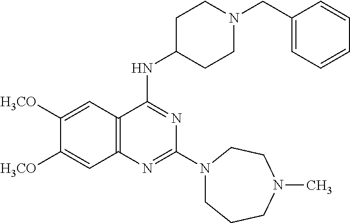 [SMILES: COc1cc2nc(N3CCCN(C)CC3)nc(NC3CCN(Cc4ccccc4)CC3)c2cc1OC] |
| UNC0224; 7-[3- (Dimethylamino)propoxy]-2- (hexahydro-4-methyl-1H-1,4- diazepin-1-yl)-6-methoxy-N-(1- methyl-4-piperidinyl)-4- quinazolinamine | 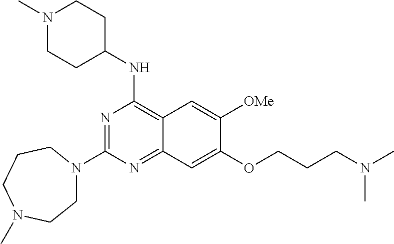 |
| UNC0321; 7-(2-(2- (Dimethylamino)ethoxy)ethoxy)-6- methoxy-2-(4-methyl-1,4-diazepan- 1-yl)-N-(1-methylpiperidin-4- yl)quinazolin-4-amine (trifluoroacetate salt version is also available as shown here) | 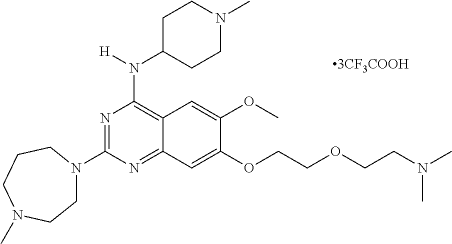 |
| UNC0638; 2-Cyclohexyl-6- methoxy-N-[1-(1-methylethyl)-4- piperidinyl]-7-[3-(1- pyrrolidinyl)propoxy]-4- quinazolinamine | 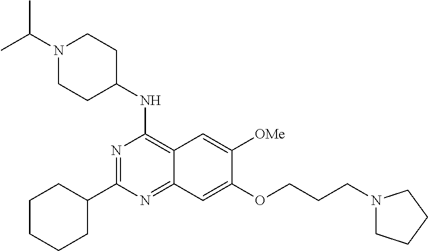 [SMILES: COc1cc2c(NC3CCN(C(C)C)CC3)nc(C3CCCCC3)nc2cc1OCCCN1CCCC1] |
| UNC0646; N-(1-Cyclohexyl-4- piperidinyl)-2-[hexahydro-4-(1- methylethyl)-1H-1,4-diazepin-1-yl]- 6-methoxy-7-[3-(1- piperidinyl)propoxy]-4- quinazolinamine | 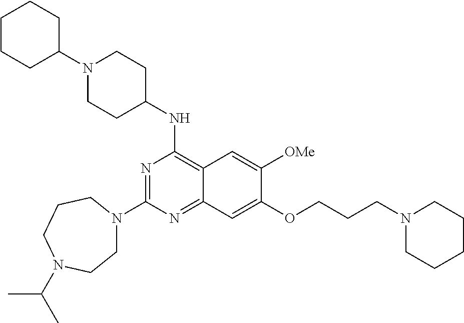 [SMILES: COc1cc2c(NC3CCN(C4CCCCC4)CC3)nc(N3CCCN(C(C)C)CC3)nc2cc1OCCCN1CCCCC1] |
| UNC0631; N-(1- (cyclohexylmethyl)piperidin-4-yl)-2- (4-isopropyl-1,4-diazepan-1-yl)-6- methoxy-7-(3-(piperidin-1- yl)propoxy)quinazolin-4-amine | 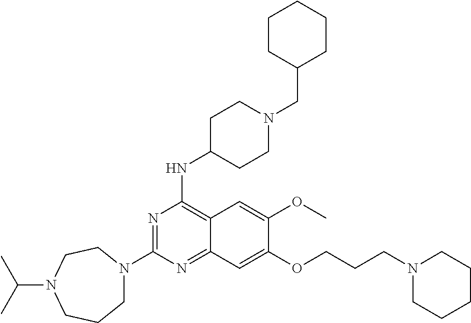 [SMILES: COc1cc2c(NC3CCN(CC4CCCCC4)CC3)nc(N3CCCN(C(C)C)CC3)nc2cc1OCCCN1CCCCC1] |
| TM2-115 | 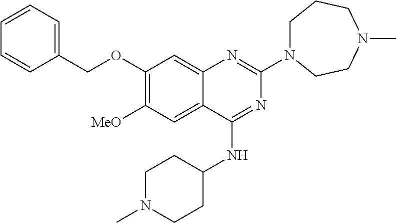 [SMILES: COc1cc2c(NC3CCN(C)CC3)nc(N3CCCN(C)CC3)nc2cc1OCc1ccccc1] |
| UNC0642; 2-(4,4-difluoropiperidin- 1-yl)-6-methoxy-N-[1-(propan-2- yl)piperidin-4-yl]-7-[3-(pyrrolidin-1- yl)propoxy]quinazolin-4-amine | 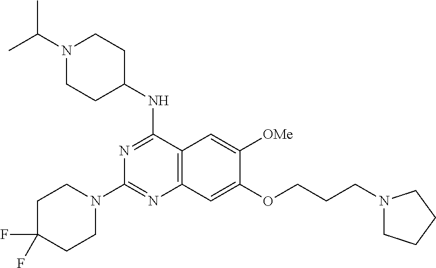 [SMILES: COc1cc2c(NC3CCN(C(C)C)CC3)nc(N3CCC(F)(F)CC3)nc2cc1OCCCN1CCCC1] |
| BIX-01338 | 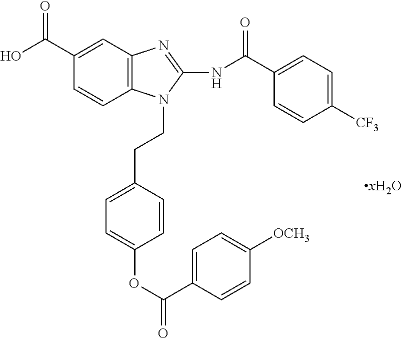 [SMILES: COc1ccc(C(=O)Oc2ccc(CCn3c(NC(=O)c4ccc(C(F)(F)F)cc4)nc4cc(C(=O)O)ccc43)cc2)cc1] |
| E72 | 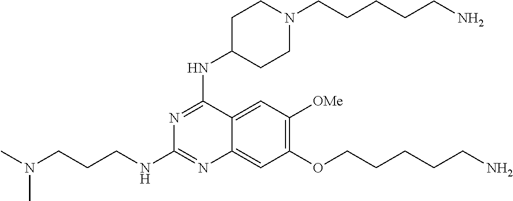 [SMILES: COc1cc2c(NC3CCN(CCCCCN)CC3)nc(NCCCN(C)C)nc2cc1OCCCCCN] |
| A-366 | 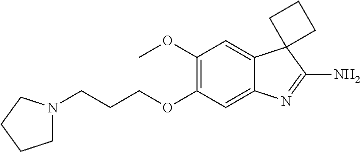 [SMILES: COc1cc2c(cc1OCCCN1CCCC1)N=C(N)C21CCC1] |
| BRD4770; 2-(Benzoylamino)-1-(3- phenylpropyl)-1H-benzimidazole-5- carboxylic acid methyl ester | 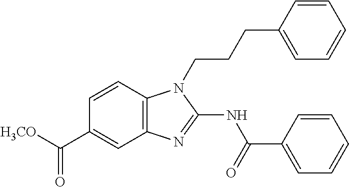 [SMILES: COC(=O)c1ccc2c(c1)nc(NC(=O)c1ccccc1)n2CCCc1ccccc1] |
| Sinefungin | 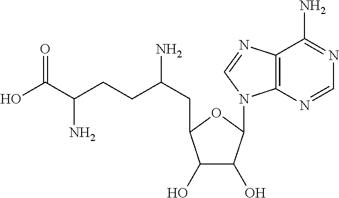 [SMILES: Nc1ncnc2c1ncn2C1OC(CC(N)CCC(N)C(=O)O)C(O)C1O] |
| Sinefungin analogue: 5′-desoxy-5′-ethyladenosine | 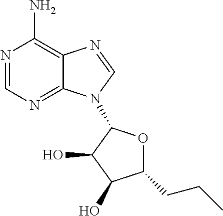 [SMILES: CCC[C@H]1O[C@@H](n2cnc3c(N)ncnc32)[C@H](O)[C@@H]1O] |
| Sinefungin analogue: 5′-desoxy-5′-butyladenosine | 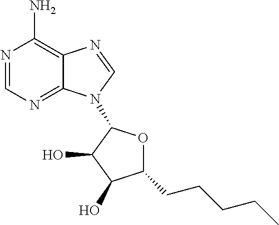 [SMILES: CCCCC[C@H]1O[C@@H](n2cnc3c(N)ncnc32)[C@H](O)[C@@H]1O] |
| Sinefungin analogue: 5′-desoxy-5′-(2- ethoxycarbonylethyl)adenosine | 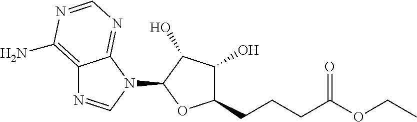 [SMILES: CCOC(=O)CCC[C@H]1O[C@@H](n2cnc3c(N)ncnc32)[C@H](O)[C@@H]1O] |
| Sinefungin analogue: 5′-desoxy-5′-(2- aminocarbonylethyl)adenosine | 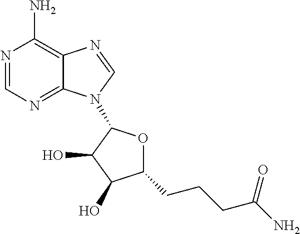 [SMILES: NC(=O)CCC[C@H]1O[C@@H](n2cnc3c(N)ncnc32)[C@H](O)[C@@H]1O] |
| Sinefungin analogue: 5′-desoxy-5′-(2-phenylethyl) adenosine | 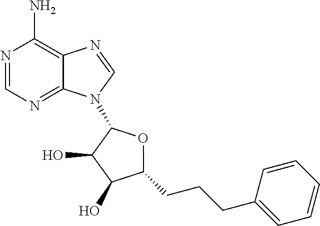 [SMILES: Nc1ncnc2c1ncn2[C@@H]1O[C@H](CCCc2ccccc2)[C@@H](O)[C@H]1O] |
| Sinefungin analogue: 5′-desoxy-5′-benzyladenosine | 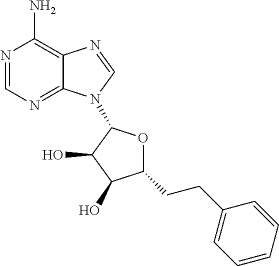 [SMILES: Nc1ncnc2c1ncn2[C@@H]1O[C@H](CCc2ccccc2)[C@@H](O)[C@H]1O] |
| Sinefungin analogue: 5′-desoxy-5′-(2- fluorobenzyl)adenosine | 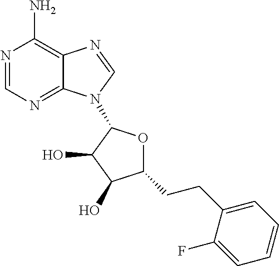 [SMILES: Nc1ncnc2c1ncn2[C@@H]1O[C@H](CCc2ccccc2F)[C@@H](O)[C@H]1O] |
| Sinefungin analogue: 5′-desoxy-5′-(4- fluorobenzyl)adenosine | 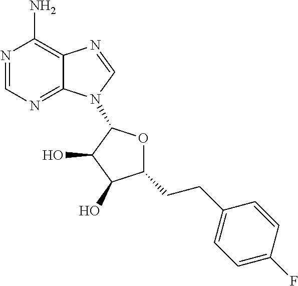 [SMILES: Nc1ncnc2c1ncn2[C@@H]1O[C@H](CCc2ccc(F)cc2)[C@@H](O)[C@H]1O] |
| Sinefungin analogue: 5′-desoxy-5′-(1- thiazolylmethyl)adenosine | 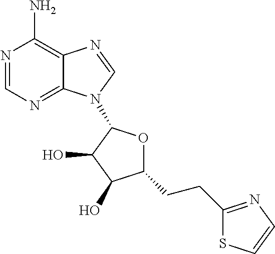 [SMILES: Nc1ncnc2c1ncn2[C@@H]1O[C@H](CCc2nccs2)[C@@H](O)[C@H]1O] |
| Sinefungin analogue: 5′-desoxy-5′-(3- phenylpropyl)adenosine | 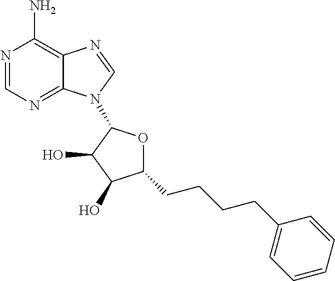 [SMILES: Nc1ncnc2c1ncn2[C@@H]1O[C@H](CCCCc2ccccc2)[C@@H](O)[C@H]1O] |
| Sinefungin analogue: 5′-desoxy-5′-(2- cyanobenzyl)adenosine | 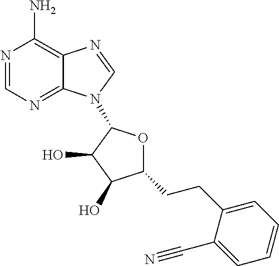 [SMILES: N#Cc1ccccc1CC[C@H]1O[C@@H](n2cnc3c(N)ncnc32)[C@H](O)[C@@H]1O] |
| Sinefungin analogue: 5′-desoxy-5′-(3″-methyl-propyl-1″)- adenosine | 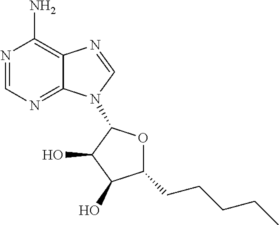 [SMILES: CCCCC[C@H]1O[C@@H](n2cnc3c(N)ncnc32)[C@H](O)[C@@H]1O] |
| Sinefungin analogue: 5′-desoxy-5′-(1″-isopropyl- 1″aminomethyl)-adenosine | 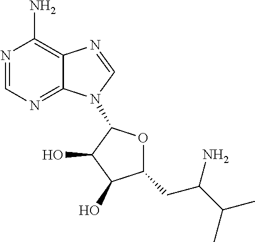 [SMILES: CC(C)C(N)C[C@H]1O[C@@H](n2cnc3c(N)ncnc32)[C@H](O)[C@@H]1O] |
| Sinefungin analogue: 5′-desoxy-5′-(2″-cyclopentyl- 1″aminoethyl)-adenosine | 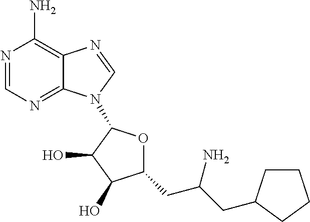 [SMILES: Nc1ncnc2c1ncn2[C@@H]1O[C@H](CC(N)CC2CCCC2)[C@@H](O)[C@H]1O] |
| Sinefungin analogue: 5′-desoxy-5′-(2″-cyclohexyl- 1″aminoethyl)-adenosine | 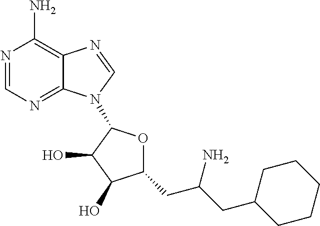 [SMILES: Nc1ncnc2c1ncn2[C@@H]1O[C@H](CC(N)CC2CCCCC2)[C@@H](O)[C@H]1O] |
| Sinefungin analogue: 5′-desoxy-5′-(2″-3,4- dimethoxyphenyl-1″aminoethyl)- adenosine | 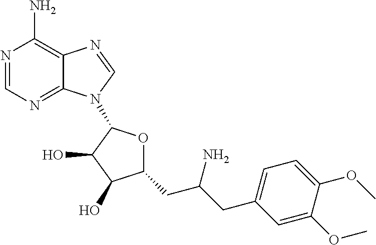 [SMILES: COc1ccc(CC(N)C[C@H]2O[C@@H](n3cnc4c(N)ncnc43)[C@H](O)[C@@H]2O)cc1OC] |
| Sinefungin analogue: 5′-desoxy-5′-(2″-4-ethylphenyl- 1″aminoethyl)adenosine | 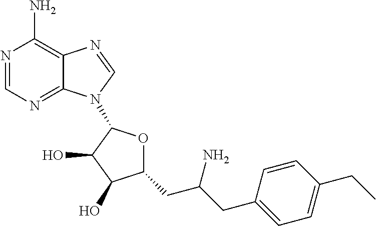 [SMILES: CCc1ccc(CC(N)C[C@H]2O[C@@H](n3cnc4c(N)ncnc43)[C@H](O)[C@@H]2O)cc1] |
| Sinefungin analogue: 5′-desoxy-5′-(1″-cyclohexyl- 1″aminomethyl)-adenosine | 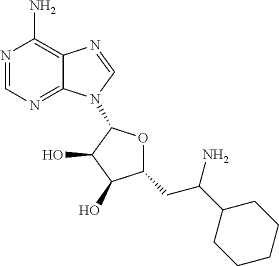 [SMILES: Nc1ncnc2c1ncn2[C@@H]1O[C@H](CC(N)C2CCCCC2)[C@@H](O)[C@H]1O] |

### Route of Delivery

The compositions (e.g., oligonucleotides, expression vectors, inhibitors) described herein can be delivered to a subject by a variety of routes. Exemplary routes include: intrathecal, intraneural, intracerebral, intramuscular, oral, intravenous, intradermal, topical, rectal, parenteral, anal, intravaginal, intranasal, pulmonary, or ocular. The term “therapeutically effective amount” is the amount of active agent (e.g., oligonucleotide, expression vector, inhibitor) present in the composition that is needed to provide the desired level of FXN expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The oligonucleotides, expression vectors, and inhibitors of the invention can be incorporated into pharmaceutical compositions suitable for administration. Such compositions typically include one or more species of oligonucleotide, expression vector, or inhibitor and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the composition in aerosol form. The vascular endothelial cells could be targeted by coating a balloon catheter with the composition and mechanically introducing the composition. Targeting of neuronal cells could be accomplished by intrathecal, intraneural, intracerebral administration.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver compositions to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g., neuronal tissue, neuromuscular tissue).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as asorbic acid, EDTA or benzylchronium chloride, and the usual quantities of diluents and/or carriers. The oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. A oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide.

In one embodiment, unit doses or measured doses of a composition that includes oligonucleotide are dispensed by an implanted device. The device can include a sensor that monitors a parameter within a subject. For example, the device can include pump, e.g., and, optionally, associated electronics.

Tissue, e.g., cells or organs can be treated with an oligonucleotide or expression vector, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, the oligonucleotide or expression vector treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

### Dosage

In one aspect, the invention features a method of administering an oligonucleotide, expression vector, or inhibitor to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with a reduced level of FXN. The unit dose, for example, can be administered by injection (e.g., intrathecal, intraneural, intracerebral, intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of an oligonucleotide, expression vector, or inhibitor. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, a pharmaceutical composition includes a plurality of active species (e.g, a plurality of oligonucleotides, expression vectors and/or inhibitors). In some embodiment, an oligonucleotide species has sequences that are non-overlapping and non-adjacent to another oligonucleotide species with respect to a target sequence (e.g., an mRNA of a negative epigenetic regulator of FXN). In another embodiment, the plurality of oligonucleotide species is specific for different mRNAs of different negative epigenetic regulators of FXN. In another embodiment, the oligonucleotide is allele specific.

In some cases, a patient is treated with an oligonucleotide, expression vector, or inhibitor in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of the oligonucleotide or inhibitor composition is an amount sufficient to be effective in treating or preventing a disorder or to regulate a physiological condition in humans. The concentration or amount of oligonucleotide or inhibitor administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide and/or inhibitor can include a single treatment or, preferably, can include a series of treatments. It will also be appreciated that the effective dosage of an oligonucleotide and/or inhibitor used for treatment may increase or decrease over the course of a particular treatment. For example, the subject can be monitored after administering an oligonucleotide or inhibitor composition. Based on information from the monitoring, an additional amount of the oligonucleotide and/or inhibitor composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of FXN expression levels in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human FXN and/or a human negative epigenetic regulator of FXN. In another embodiment, a composition for testing in an animal model includes an oligonucleotide that is complementary, at least in an internal region, to a sequence that is conserved between an mRNA of a negative epigenetic regulator of FXN in the animal model and the mRNA of the negative epigenetic regulator of FXN in a human.

In one embodiment, the administration of a composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, ocular, intraneuronal, intrathecal, or intracerebral. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising an oligonucleotide, expression vector, or inhibitor. In some embodiments, the composition is a pharmaceutical composition comprising an oligonucleotide, expression vector, or inhibitor and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for oligonucleotides or inhibitors, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting.

### EXAMPLES

### Example 1

### Knockdown of Epigenetic Factors and FXN Expression

### Introduction

An RNAi based genetic screen was performed in cells from FRDA patients to identify regulators of FXN. Several genes were identified as being negative regulators of FXN expression. When expression of these negative regulators is knocked down in cells, FXN expression increases in the cells. Several other genes were identified as being positive regulators of FXN expression. When expression of these positive regulators is knocked down in the cells, FXN expression decreases in the cells. Thus, described herein are certain regulatory factors that modulate expression of FXN in cells.

### Materials and Methods:

siRNA Screen An siRNA screen was performed in the GM03816 cell line, which is a fibroblast cell line from a patient with Friedriech's ataxia (FRDA). Cells were treated with the Human Epigenetics siGENOME® SMARTpool® siRNA Library (Dharmacon) according to the manufacturer's instructions. RNA was harvested (at day 4 after treatment) and real time PCR performed to measure the level of FXN mRNA after treatment of the cells with the siRNA library.

### Real Time PCR

RNA was harvested from the cells using Promega SV 96 Total RNA Isolation system or Trizol omitting the DNAse step. RNA harvested from cells was normalized so that 50 ng of RNA was input to each reverse transcription reaction. For the few samples that were too dilute to reach this limit, the maximum input volume was added. Reverse transcriptase reaction was performed using the Superscript II kit and real time PCR performed on cDNA samples using icycler SYBR green chemistry (Biorad). A baseline level of mRNA expression for each target gene was determined through quantitative PCR as outlined above. Baseline levels were also determined for mRNA of various housekeeping genes which are constitutively expressed. A “control” housekeeping gene with approximately the same level of baseline expression as the target gene was chosen for comparison purposes.

### Cell Lines

Cells were cultured using conditions known in the art (see, e.g., Current Protocols in Cell Biology). Details of the cell lines used in the experiments described herein are provided in Table 3.

TABLE 3
Cell lines

| | Clinically | | # of GAA | |
|---|---|---|---|---|
| Cell lines | affected | Cell type | repeats | Notes |
| GM03816 | Yes | Fibroblast | 330/380 | Coriell Cell |
| | | | | Repository |

### Pathway Enrichment Analysis

Genes identified in the siRNA screen that caused greater than two-fold upregulation or downregulation of FXN mRNA were analyzed using the Database for Annotation, Visualization and Integrated Discovery (DAVID, available to through DAVID Bioinformatics Resources website) to identify pathways that were enriched in the gene set. The Functional Annotation DAVID tool was used to perform the enrichment analysis.

### Oligonucleotide Design

Oligonucleotides were designed to target a subset of the genes identified in the siRNA screen. The sequence and structure of each oligonucleotide is shown in Table 4. Table 5 provides a description of the nucleotide analogs, modifications and internucleoside linkages used for certain oligonucleotides described in Table 4.

TABLE 4
Oligonucleotides designed to target negative epigenetic regulators of FXN

| SEQ | Oligo | | Gene | | |
|---|---|---|---|---|---|
| ID NO | Name | Base Sequence | Name | Organism | Formatted Sequence |
| 1 | ACTL6 | GGCAACAAAGCGGC | ACTL6A | human | InaGs;InaGs;InaCs;dAs;dAs;dCs;dAs;dAs;dAs; |
| | A-01 | G | | | dGs;dCs;dGs;InaGs;InaCs;InaG-Sup |
| | m08 | | | | |
| 2 | ACTL6 | ATCGCCATCTATTTC | ACTL6A | human | InaAs;InaTs;InaCs;dGs;dCs;dCs;dAs;dTs;dCs; |
| | A-02 | | | | dTs;dAs;dTs;InaTs;InaTs;InaC-Sup |
| | m08 | | | | |
| 3 | ACTL6 | GAACGACCATTAGC | ACTL6A | human | InaGs;InaAs;InaAs;dCs;dGs;dAs;dCs;dCs;dAs; |
| | A-03 | A | | | dTs;dTs;dAs;InaGs;InaCs;InaA-Sup |
| | m08 | | | | |
| 4 | ACTL6 | GCCCAGTAGAACGA | ACTL6A | human | InaGs;InaCs;InaCs;dCs;dAs;dGs;dTs;dAs;dGs; |
| | A-04 | C | | | dAs;dAs;dCs;InaGs;InaAs;InaC-Sup |
| | m08 | | | | |
| 5 | ACTL6 | CATCGTGGACTGGA | ACTL6A | human | InaCs;InaAs;InaTs;dCs;dGs;dTs;dGs;dGs;dAs; |
| | A-05 | A | | | dCs;dTs;dGs;InaGs;InaAs;InaA-Sup |
| | m08 | | | | |
| 6 | ACTL6 | TTTCAACCGCATACT | ACTL6A | human | InaTs;InaTs;InaTs;dCs;dAs;dAs;dCs;dCs;dGs; |
| | A-06 | | | | dCs;dAs;dTs;InaAs;InaCs;InaT-Sup |
| | m08 | | | | |
| 7 | ACTL6 | GAGCTAAACCTCCGT | ACTL6A | human | InaGs;InaAs;InaGs;dCs;dTs;dAs;dAs;dAs;dCs; |
| | A-07 | | | | dCs;dTs;dCs;InaCs;InaGs;InaT-Sup |
| | m08 | | | | |
| 8 | ACTL6 | GAGCCGCCAATCCA | ACTL6A | human | InaGs;InaAs;InaGs;dCs;dCs;dGs;dCs;dCs;dAs; |
| | A-08 | T | | | dAs;dTs;dCs;InaCs;InaAs;InaT-Sup |
| | m08 | | | | |
| 9 | EID1- | AAATTCCTCGCCCTC | EID1 | human | InaAs;InaAs;InaAs;dTs;dTs;dCs;dCs;dTs;dCs; |
| | 01 | | | | dGs;dCs;dCs;InaCs;InaTs;InaC-Sup |
| | m08 | | | | |
| 10 | EID1- | CGTAGTCGTCCTCCC | EID1 | human | InaCs;InaGs;InaTs;dAs;dGs;dTs;dCs;dGs;dTs; |
| | 02 | | | | dCs;dCs;dTs;InaCs;InaCs;InaC-Sup |
| | m08 | | | | |
| 11 | EID1- | CTGAAACCCGCCATC | EID1 | human | InaCs;InaTs;InaGs;dAs;dAs;dAs;dCs;dCs;dCs; |
| | 03 | | | | dGs;dCs;dCs;InaAs;InaTs;InaC-Sup |
| | m08 | | | | |
| 12 | EID1- | AGCTCTTCGATAAAA | EID1 | human | InaAs;InaGs;InaCs;dTs;dCs;dTs;dTs;dCs;dGs; |
| | 04 | | | | dAs;dTs;dAs;InaAs;InaAs;InaA-Sup |
| | m08 | | | | |
| 13 | EID1- | TCGGTCAGACGATT | EID1 | human | InaTs;InaCs;InaGs;dGs;dTs;dCs;dAs;dGs;dAs; |
| | 05 | G | | | dCs;dGs;dAs;InaTs;InaTs;InaG-Sup |
| | m08 | | | | |
| 14 | EID1- | CTCATCACAGCCGA | EID1 | human | InaCs;InaTs;InaCs;dAs;dTs;dCs;dAs;dCs;dAs; |
| | 06 | G | | | dGs;dCs;dCs;InaGs;InaAs;InaG-Sup |
| | m08 | | | | |
| 15 | IDH1- | ACGATTCTCTATGCC | IDH1 | human | InaAs;InaCs;InaGs;dAs;dTs;dTs;dCs;dTs;dCs; |
| | 01 | | | | dTs;dAs;dTs;InaGs;InaCs;InaC-Sup |
| | m08 | | | | |
| 16 | IDH1- | TGGCATCACGATTCT | IDH1 | human | InaTs;InaGs;InaGs;dCs;dAs;dTs;dCs;dAs;dCs; |
| | 02 | | | | dGs;dAs;dTs;InaTs;InaCs;InaT-Sup |
| | m08 | | | | |
| 17 | IDH1- | TCAATTGACTTATCT | IDH1 | human | InaTs;InaCs;InaAs;dAs;dTs;dTs;dGs;dAs;dCs; |
| | 03 | | | | dTs;dTs;dAs;InaTs;InaCs;InaT-Sup |
| | m08 | | | | |
| 18 | IDH1- | ACGCCCATCATATTT | IDH1 | human | InaAs;InaCs;InaGs;dCs;dCs;dCs;dAs;dTs;dCs; |
| | 04 | | | | dAs;dTs;dAs;InaTs;InaTs;InaT-Sup |
| | m08 | | | | |
| 19 | IDH1- | TGTCTTTAAAACGCC | IDH1 | human | InaTs;InaGs;InaTs;dCs;dTs;dTs;dTs;dAs;dAs; |
| | 05 | | | | dAs;dAs;dCs;InaGs;InaCs;InaC-Sup |
| | m08 | | | | |
| 20 | IDH1- | TTATCAAGCTTTGCT | IDH1 | human | InaTs;InaTs;InaAs;dTs;dCs;dAs;dAs;dGs;dCs; |
| | 06 | | | | dTs;dTs;dTs;InaGs;InaCs;InaT-Sup |
| | m08 | | | | |
| 21 | JAK2- | GTCATCGTAAGGCA | JAK2 | human | InaGs;InaTs;InaCs;dAs;dTs;dCs;dGs;dTs;dAs; |
| | 01 | G | | | dAs;dGs;dGs;InaCs;InaAs;InaG-Sup |
| | m08 | | | | |
| 22 | JAK2- | GGATCTTTGCTCGAA | JAK2 | human | InaGs;InaGs;InaAs;dTs;dCs;dTs;dTs;dTs;dGs; |
| | 02 | | | | dCs;dTs;dCs;InaGs;InaAs;InaA-Sup |
| | m08 | | | | |
| 23 | JAK2- | TAGTCTTGGATCTTT | JAK2 | human | InaTs;InaAs;InaGs;dTs;dCs;dTs;dTs;dGs;dGs; |
| | 03 | | | | dAs;dTs;dCs;InaTs;InaTs;InaT-Sup |
| | m08 | | | | |
| 24 | JAK2- | TGCGAAATCTGTACC | JAK2 | human | InaTs;InaGs;InaCs;dGs;dAs;dAs;dAs;dTs;dCs; |
| | 04 | | | | dTs;dGs;dTs;InaAs;InaCs;InaC-Sup |
| | m08 | | | | |
| 25 | JAK2- | TGAATTCCACCGTTT | JAK2 | human | InaTs;InaGs;InaAs;dAs;dTs;dTs;dCs;dCs;dAs; |
| | 05 | | | | dCs;dCs;dGs;InaTs;InaTs;InaT-Sup |
| | m08 | | | | |
| 26 | JAK2- | ATCGCAATATAACTG | JAK2 | human | InaAs;InaTs;InaCs;dGs;dCs;dAs;dAs;dTs;dAs; |
| | 06 | | | | dTs;dAs;dAs;InaCs;InaTs;InaG-Sup |
| | m08 | | | | |
| 27 | JAK2- | TGACATTTTCTCGCT | JAK2 | human | InaTs;InaGs;InaAs;dCs;dAs;dTs;dTs;dTs;dTs; |
| | 07 | | | | dCs;dTs;dCs;InaGs;InaCs;InaT-Sup |
| | m08 | | | | |
| 28 | JAK2- | TCATACCGGCACATC | JAK2 | human | InaTs;InaCs;InaAs;dTs;dAs;dCs;dCs;dGs;dGs; |
| | 08 | | | | dCs;dAs;dCs;InaAs;InaTs;InaC-Sup |
| | m08 | | | | |
| 29 | JAK2- | GTCTCGTAAACTTCC | JAK2 | human | InaGs;InaTs;InaCs;dTs;dCs;dGs;dTs;dAs;dAs; |
| | 09 | | | | dAs;dCs;dTs;InaTs;InaCs;InaC-Sup |
| | m08 | | | | |
| 30 | JAK2- | TGATCTATCCGTTCT | JAK2 | human | InaTs;InaGs;InaAs;dTs;dCs;dTs;dAs;dTs;dCs; |
| | 10 | | | | dCs;dGs;dTs;InaTs;InaCs;InaT-Sup |
| | m08 | | | | |
| 31 | KAT2A- | AGGTCGAGCCGGAT | KAT2A | human | InaAs;InaGs;InaGs;dTs;dCs;dGs;dAs;dGs;dCs; |
| | 01 | C | | | dCs;dGs;dGs;InaAs;InaTs;InaC-Sup |
| | m08 | | | | |
| 32 | KAT2A- | CCGGACTTGCGCCTT | KAT2A | human | InaCs;InaCs;InaGs;dGs;dAs;dCs;dTs;dTs;dGs; |
| | 02 | | | | dCs;dGs;dCs;InaCs;InaTs;InaT-Sup |
| | m08 | | | | |
| 33 | KAT2A- | TGAGAGCTCGAACA | KAT2A | human | InaTs;InaGs;InaAs;dGs;dAs;dGs;dCs;dTs;dCs; |
| | 03 | T | | | dGs;dAs;dAs;InaCs;InaAs;InaT-Sup |
| | m08 | | | | |
| 34 | KAT2A- | CACGGAGCCGCTTG | KAT2A | human | InaCs;InaAs;InaCs;dGs;dGs;dAs;dGs;dCs;dCs; |
| | 04 | G | | | dGs;dCs;dTs;InaTs;InaGs;InaG-Sup |
| | m08 | | | | |
| 35 | KAT2A- | CATTGACCAGCTCCA | KAT2A | human | InaCs;InaAs;InaTs;dTs;dGs;dAs;dCs;dCs;dAs; |
| | 05 | | | | dGs;dCs;dTs;InaCs;InaCs;InaA-Sup |
| | m08 | | | | |
| 36 | KAT2A- | GGCGATATACTCCTT | KAT2A | human | InaGs;InaGs;InaCs;dGs;dAs;dTs;dAs;dTs;dAs; |
| | 06 | | | | dCs;dTs;dCs;InaCs;InaTs;InaT-Sup |
| | m08 | | | | |
| 37 | KAT2A- | CACCGATGACCCGC | KAT2A | human | InaCs;InaAs;InaCs;dCs;dGs;dAs;dTs;dGs;dAs; |
| | 07 | C | | | dCs;dCs;dCs;InaGs;InaCs;InaC-Sup |
| | m08 | | | | |
| 38 | KAT2A- | CCATCAGCGTCGCTC | KAT2A | human | InaCs;InaCs;InaAs;dTs;dCs;dAs;dGs;dCs;dGs; |
| | 08 | | | | dTs;dCs;dGs;InaCs;InaTs;InaC-Sup |
| | m08 | | | | |
| 39 | KAT2A- | CTGTTTGCGCTCAAT | KAT2A | human | InaCs;InaTs;InaGs;dTs;dTs;dTs;dGs;dCs;dGs; |
| | 09 | | | | dCs;dTs;dCs;InaAs;InaAs;InaT-Sup |
| | m08 | | | | |
| 40 | KAT2A- | GGCGATGACCCGCT | KAT2A | human | InaGs;InaGs;InaCs;dGs;dAs;dTs;dGs;dAs;dCs; |
| | 10 | G | | | dCs;dCs;dGs;InaCs;InaTs;InaG-Sup |
| | m08 | | | | |
| 41 | PRKCD- | CATGGTCGGCTTCTT | PRKCD | human | InaCs;InaAs;InaTs;dGs;dGs;dTs;dCs;dGs;dGs; |
| | 01 | | | | dCs;dTs;dTs;InaCs;InaTs;InaT-Sup |
| | m08 | | | | |
| 42 | PRKCD- | TGCGCATAGACTGTT | PRKCD | human | InaTs;InaGs;InaCs;dGs;dCs;dAs;dTs;dAs;dGs; |
| | 02 | | | | dAs;dCs;dTs;InaGs;InaTs;InaT-Sup |
| | m08 | | | | |
| 43 | PRKCD- | GGTGGCGATAAACT | PRKCD | human | InaGs;InaGs;InaTs;dGs;dGs;dCs;dGs;dAs;dTs; |
| | 03 | C | | | dAs;dAs;dAs;InaCs;InaTs;InaC-Sup |
| | m08 | | | | |
| 44 | PRKCD- | ATCTTGTCGATGCAT | PRKCD | human | InaAs;InaTs;InaCs;dTs;dTs;dGs;dTs;dCs;dGs; |
| | 04 | | | | dAs;dTs;dGs;InaCs;InaAs;InaT-Sup |
| | m08 | | | | |
| 45 | PRKCD- | TGTTGAAGCGTTCTT | PRKCD | human | InaTs;InaGs;InaTs;dTs;dGs;dAs;dAs;dGs;dCs; |
| | 05 | | | | dGs;dTs;dTs;InaCs;InaTs;InaT-Sup |
| | m08 | | | | |
| 46 | PRKCD- | CGATGTTGAAGCGT | PRKCD | human | InaCs;InaGs;InaAs;dTs;dGs;dTs;dTs;dGs;dAs; |
| | 06 | T | | | dAs;dGs;dCs;InaGs;InaTs;InaT-Sup |
| | m08 | | | | |
| 47 | PRKCD- | AAGCGGCCTTTGTCC | PRKCD | human | InaAs;InaAs;InaGs;dCs;dGs;dGs;dCs;dCs;dTs; |
| | 07 | | | | dTs;dTs;dGs;InaTs;InaCs;InaC-Sup |
| | m08 | | | | |
| 48 | PRKCD- | TAGAGTTCAAAGCG | PRKCD | human | InaTs;InaAs;InaGs;dAs;dGs;dTs;dTs;dCs;dAs; |
| | 08 | G | | | dAs;dAs;dGs;InaCs;InaGs;InaG-Sup |
| | m08 | | | | |
| 49 | PRKCD- | CCCCGAAAGACCAC | PRKCD | human | InaCs;InaCs;InaCs;dCs;dGs;dAs;dAs;dAs;dGs; |
| | 09 | C | | | dAs;dCs;dCs;InaAs;InaCs;InaC-Sup |
| | m08 | | | | |
| 50 | PRKCD- | CACGGATGGACTCG | PRKCD | human | InaCs;InaAs;InaCs;dGs;dGs;dAs;dTs;dGs;dGs; |
| | 10 | A | | | dAs;dCs;dTs;InaCs;InaGs;InaA-Sup |
| | m08 | | | | |
| 51 | PRKCD- | AGTCGATGAGGTTC | PRKCD | human | InaAs;InaGs;InaTs;dCs;dGs;dAs;dTs;dGs;dAs; |
| | 11 | T | | | dGs;dGs;dTs;InaTs;InaCs;InaT-Sup |
| | m08 | | | | |
| 52 | TNFSF9- | GTCAGAGGCGTATT | TNFSF9 | human | InaGs;InaTs;InaCs;dAs;dGs;dAs;dGs;dGs;dCs; |
| | 01 | C | | | dGs;dTs;dAs;InaTs;InaTs;InaC-Sup |
| | m08 | | | | |
| 53 | TNFSF9- | AGCAGCCCCGCGAC | TNFSF9 | human | InaAs;InaGs;InaCs;dAs;dGs;dCs;dCs;dCs;dCs; |
| | 02 | C | | | dGs;dCs;dGs;InaAs;InaCs;InaC-Sup |
| | m08 | | | | |
| 54 | TNFSF9- | GACGGCGCAGGCGG | TNFSF9 | human | InaGs;InaAs;InaCs;dGs;dGs;dCs;dGs;dCs;dAs |
| | 03 | C | | | ;dGs;dGs;dCs;InaGs;InaGs;InaC-Sup |
| | m08 | | | | |
| 55 | TNFSF9- | CTGAGCCCTCGCCG | TNFSF9 | human | InaCs;InaTs;InaGs;dAs;dGs;dCs;dCs;dCs;dTs; |
| | 04 | G | | | dCs;dGs;dCs;InaCs;InaGs;InaG-Sup |
| | m08 | | | | |
| 56 | TNFSF9- | GGTCCACGGTCAAA | TNFSF9 | human | InaGs;InaGs;InaTs;dCs;dCs;dAs;dCs;dGs;dGs; |
| | 05 | G | | | dTs;dCs;dAs;InaAs;InaAs;InaG-Sup |
| | m08 | | | | |
| 57 | TNFSF9- | AAACCGAAGGCCGA | TNFSF9 | human | InaAs;InaAs;InaAs;dCs;dCs;dGs;dAs;dAs;dGs; |
| | 06 | G | | | dGs;dCs;dCs;InaGs;InaAs;InaG-Sup |
| | m08 | | | | |
| 58 | TNFSF9- | AGGTGCAGCAAGCG | TNFSF9 | human | InaAs;InaGs;InaGs;dTs;dGs;dCs;dAs;dGs;dCs; |
| | 07 | G | | | dAs;dAs;dGs;InaCs;InaGs;InaG-Sup |
| | m08 | | | | |
| 59 | TNFSF9- | GTCACCCGGAAGAG | TNFSF9 | human | InaGs;InaTs;InaCs;dAs;dCs;dCs;dCs;dGs;dGs; |
| | 08 | T | | | dAs;dAs;dGs;InaAs;InaGs;InaT-Sup |
| | m08 | | | | |
| 60 | TNFSF9- | AGTAGGATTCGGAC | TNFSF9 | human | InaAs;InaGs;InaTs;dAs;dGs;dGs;dAs;dTs;dTs; |
| | 09 | T | | | dCs;dGs;dGs;InaAs;InaCs;InaT-Sup |
| | m08 | | | | |
| 61 | JUND- | CCGTAGAAGGGTGT | JUND | human | InaCs;InaCs;InaGs;dTs;dAs;dGs;dAs;dAs;dGs; |
| | 01 | T | | | dGs;dGs;dTs;InaGs;InaTs;InaT-Sup |
| | m08 | | | | |
| 62 | JUND- | TTCATCATGCTGCCG | JUND | human | InaTs;InaTs;InaCs;dAs;dTs;dCs;dAs;dTs;dGs; |
| | 02 | | | | dCs;dTs;dGs;InaCs;InaCs;InaG-Sup |
| | m08 | | | | |
| 63 | JUND- | CTGTGAGCTCGTCG | JUND | human | InaCs;InaTs;InaGs;dTs;dGs;dAs;dGs;dCs;dTs; |
| | 03 | G | | | dCs;dGs;dTs;InaCs;InaGs;InaG-Sup |
| | m08 | | | | |
| 64 | JUND- | GGAACTGTGAGCTC | JUND | human | InaGs;InaGs;InaAs;dAs;dCs;dTs;dGs;dTs;dGs; |
| | 04 | G | | | dAs;dGs;dCs;InaTs;InaCs;InaG-Sup |
| | m08 | | | | |
| 65 | JUND- | GCTCGTCCTTGAGCG | JUND | human | InaGs;InaCs;InaTs;dCs;dGs;dTs;dCs;dCs;dTs; |
| | 05 | | | | dTs;dGs;dAs;InaGs;InaCs;InaG-Sup |
| | m08 | | | | |
| 66 | JUND- | TGGCTCGTCCTTGAG | JUND | human | InaTs;InaGs;InaGs;dCs;dTs;dCs;dGs;dTs;dCs; |
| | 06 | | | | dCs;dTs;dTs;InaGs;InaAs;InaG-Sup |
| | m08 | | | | |
| 67 | JUND- | CCCGTTGGACTGGA | JUND | human | InaCs;InaCs;InaCs;dGs;dTs;dTs;dGs;dGs;dAs; |
| | 07 | T | | | dCs;dTs;dGs;InaGs;InaAs;InaT-Sup |
| | m08 | | | | |
| 68 | JUND- | CGCTCCGCCTTGATG | JUND | human | InaCs;InaGs;InaCs;dTs;dCs;dCs;dGs;dCs;dCs; |
| | 08 | | | | dTs;dTs;dGs;InaAs;InaTs;InaG-Sup |
| | m08 | | | | |
| 69 | JUND- | CACCTGCTCGCGCA | JUND | human | InaCs;InaAs;InaCs;dCs;dTs;dGs;dCs;dTs;dCs; |
| | 09 | G | | | dGs;dCs;dGs;InaCs;InaAs;InaG-Sup |
| | m08 | | | | |
| 70 | HIC1- | GGCCGGTGTAGATG | HIC1 | human | InaGs;InaGs;InaCs;dCs;dGs;dGs;dTs;dGs;dTs; |
| | 01 | A | | | dAs;dGs;dAs;InaTs;InaGs;InaA-Sup |
| | m08 | | | | |
| 71 | HIC1- | TGACCGCGGCCTCT | HIC1 | human | InaTs;InaGs;InaAs;dCs;dCs;dGs;dCs;dGs;dGs; |
| | 02 | G | | | dCs;dCs;dTs;InaCs;InaTs;InaG-Sup |
| | m08 | | | | |
| 72 | HIC1- | TTGACCGCGGCCTCT | HIC1 | human | InaTs;InaTs;InaGs;dAs;dCs;dCs;dGs;dCs;dGs; |
| | 03 | | | | dGs;dCs;dCs;InaTs;InaCs;InaT-Sup |
| | m08 | | | | |
| 73 | HIC1- | TACCGGTCTCCTCGC | HIC1 | human | InaTs;InaAs;InaCs;dCs;dGs;dGs;dTs;dCs;dTs; |
| | 04 | | | | dCs;dCs;dTs;InaCs;InaGs;InaC-Sup |
| | m08 | | | | |
| 74 | HIC1- | ACGTACAGGTTGTC | HIC1 | human | InaAs;InaCs;InaGs;dTs;dAs;dCs;dAs;dGs;dGs; |
| | 05 | A | | | dTs;dTs;dGs;InaTs;InaCs;InaA-Sup |
| | m08 | | | | |
| 75 | HIC1- | ACACGTACAGGTTG | HIC1 | human | InaAs;InaCs;InaAs;dCs;dGs;dTs;dAs;dCs;dAs; |
| | 06 | T | | | dGs;dGs;dTs;InaTs;InaGs;InaT-Sup |
| | m08 | | | | |
| 76 | HIC1- | TCTTGTCGCACGACG | HIC1 | human | InaTs;InaCs;InaTs;dTs;dGs;dTs;dCs;dGs;dCs; |
| | 07 | | | | dAs;dCs;dGs;InaAs;InaCs;InaG-Sup |
| | m08 | | | | |
| 77 | HIC1- | AGCTCTTGTCGCACG | HIC1 | human | InaAs;InaGs;InaCs;dTs;dCs;dTs;dTs;dGs;dTs; |
| | 08 | | | | dCs;dGs;dCs;InaAs;InaCs;InaG-Sup |
| | m08 | | | | |
| 78 | HIC1- | CCGCACGCGTCGCA | HIC1 | human | InaCs;InaCs;InaGs;dCs;dAs;dCs;dGs;dCs;dGs; |
| | 09 | C | | | dTs;dCs;dGs;InaCs;InaAs;InaC-Sup |
| | m08 | | | | |
| 79 | HIC1- | TGTGCGAACTTGCC | HIC1 | human | InaTs;InaGs;InaTs;dGs;dCs;dGs;dAs;dAs;dCs; |
| | 10 | G | | | dTs;dTs;dGs;InaCs;InaCs;InaG-Sup |
| | m08 | | | | |
| 80 | HIC1- | GCTGTGCGAACTTG | HIC1 | human | InaGs;InaCs;InaTs;dGs;dTs;dGs;dCs;dGs;dAs; |
| | 11 | C | | | dAs;dCs;dTs;InaTs;InaGs;InaC-Sup |
| | m08 | | | | |
| 81 | HIC1- | TCGAGCTTGCCCTTG | HIC1 | human | InaTs;InaCs;InaGs;dAs;dGs;dCs;dTs;dTs;dGs; |
| | 12 | | | | dCs;dCs;dCs;InaTs;InaTs;InaG-Sup |
| | m08 | | | | |
| 82 | HIC1- | AGAAACGGTCGATG | HIC1 | human | InaAs;InaGs;InaAs;dAs;dAs;dCs;dGs;dGs;dTs; |
| | 13 | G | | | dCs;dGs;dAs;InaTs;InaGs;InaG-Sup |
| | m08 | | | | |
| 83 | YEATS4- | TCGGCCATTCTCTTG | YEATS4 | human | InaTs;InaCs;InaGs;dGs;dCs;dCs;dAs;dTs;dTs; |
| | 01 | | | | dCs;dTs;dCs;InaTs;InaTs;InaG-Sup |
| | m08 | | | | |
| 84 | YEATS4 | ATTCGGCCATTCTCT | YEATS4 | human | InaAs;InaTs;InaTs;dCs;dGs;dGs;dCs;dCs;dAs; |
| | -02 | | | | dTs;dTs;dCs;InaTs;InaCs;InaT-Sup |
| | m08 | | | | |
| 85 | YEATS4 | CCCGCCGGAGTCAG | YEATS4 | human | InaCs;InaCs;InaCs;dGs;dCs;dCs;dGs;dGs;dAs; |
| | -03 | G | | | dGs;dTs;dCs;InaAs;InaGs;InaG-Sup |
| | m08 | | | | |
| 86 | YEATS4- | ATATGGAGGTTTAG | YEATS4 | human | InaAs;InaTs;InaAs;dTs;dGs;dGs;dAs;dGs;dGs; |
| | 04 | T | | | dTs;dTs;dTs;InaAs;InaGs;InaT-Sup |
| | m08 | | | | |
| 87 | YEATS4- | TCGAATTCACCCCAT | YEATS4 | human | InaTs;InaCs;InaGs;dAs;dAs;dTs;dTs;dCs;dAs; |
| | 05 | | | | dCs;dCs;dCs;InaCs;InaAs;InaT-Sup |
| | m08 | | | | |
| 88 | YEATS4- | TTCGAATTCACCCCA | YEATS4 | human | InaTs;InaTs;InaCs;dGs;dAs;dAs;dTs;dTs;dCs; |
| | 06 | | | | dAs;dCs;dCs;InaCs;InaCs;InaA-Sup |
| | m08 | | | | |
| 89 | YEATS4- | TGACGAGATGTTGT | YEATS4 | human | InaTs;InaGs;InaAs;dCs;dGs;dAs;dGs;dAs;dTs; |
| | 07 | C | | | dGs;dTs;dTs;InaGs;InaTs;InaC-Sup |
| | m08 | | | | |
| 90 | YEATS4- | TAGCTGACGAGATG | YEATS4 | human | InaTs;InaAs;InaGs;dCs;dTs;dGs;dAs;dCs;dGs; |
| | 08 | T | | | dAs;dGs;dAs;InaTs;InaGs;InaT-Sup |
| | m08 | | | | |
| 91 | YEATS4- | AGTTTCACGACTTGC | YEATS4 | human | InaAs;InaGs;InaTs;dTs;dTs;dCs;dAs;dCs;dGs; |
| | 09 | | | | dAs;dCs;dTs;InaTs;InaGs;InaC-Sup |
| | m08 | | | | |

TABLE 5
Oligonucleotide Modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |

In Vitro Transfection of Cells with Oligonucleotides

Cells are seeded into each well of 24-well plates at a density of 25,000 cells per 500 uL and transfections are performed with Lipofectamine and the oligonucleotides. Control wells contain Lipofectamine alone. At time points post-transfection, approximately 200 uL of cell culture supernatants is stored at −80 C for ELISA and RNA is harvested from another aliquot of cells and quantitative PCR is carried out as outlined above. The percent induction of FXN mRNA expression by each oligonucleotide is determined by normalizing mRNA levels in the presence of the oligonucleotide to the mRNA levels in the presence of control (Lipofectamine alone).

### Results:

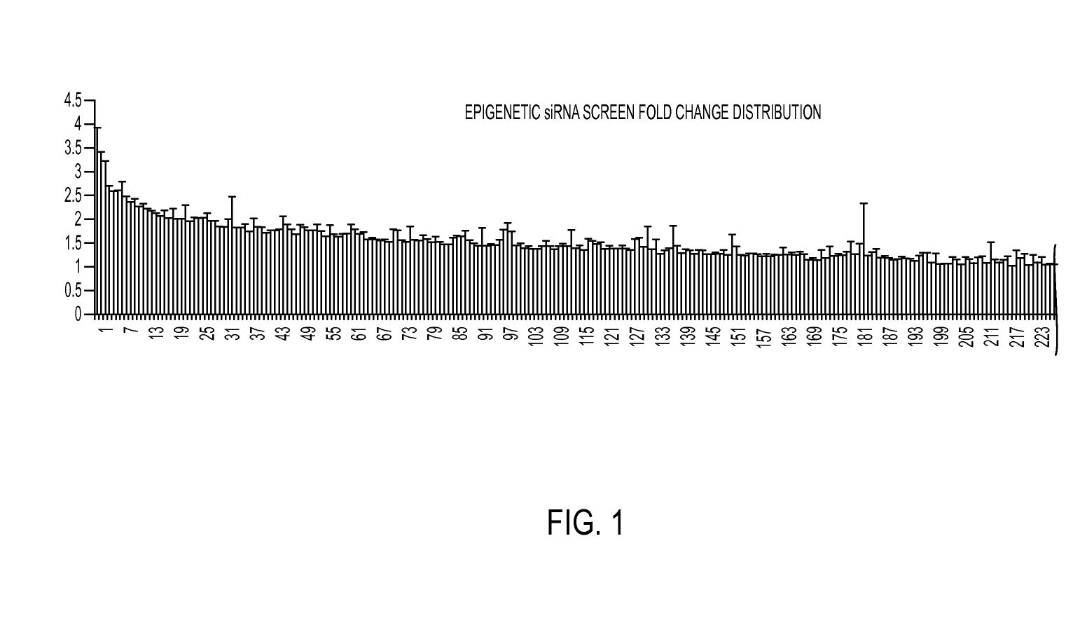

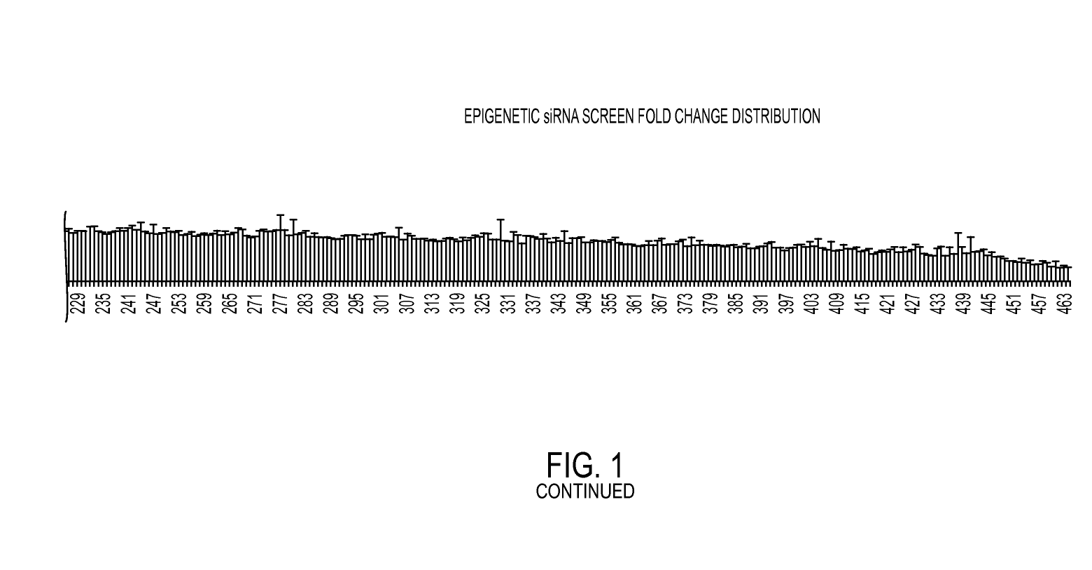

An siRNA screen was performed in FRDA fibroblasts to identify epigenetic regulators that upregulate or downregulate FXN expression when knocked down. The results of the screen are provided in Table 6 and FIG. 1 as the fold change in FXN mRNA expression compared to untreated cells. Knockdown of several epigenetic regulators caused upregulation of FXN mRNA expression, indicating that FXN expression is at least partially regulated by epigenetic factors and that some of the screened epigenetic factors are negative epigenetic regulators of FXN.

TABLE 6
siRNA evaluation results.

| | Fold | |
|---|---|---|
| Gene | Change | STDEV |
| YEATS4 | 3.987011 | 0.090224 |
| TNFSF9 | 3.296512 | 0.12244 |
| JUND | 2.990481 | 0.243108 |
| ACTL6A | 2.895875 | 0.195745 |
| HIC1 | 2.599866 | 0.120826 |
| PRKCD | 2.576589 | 0.011642 |
| MEAF6 | 2.516965 | 0.038728 |
| JAK2 | 2.441371 | 0.352924 |
| EID1 | 2.373967 | 0.121625 |
| CFTR | 2.370923 | 0.005513 |
| TADA3 | 2.329775 | 0.120086 |
| MYBL2 | 2.273287 | 0.010571 |
| KAT2A | 2.229702 | 0.102989 |
| IDH1 | 2.16832 | 0.065606 |
| EPC2 | 2.125487 | 0.065875 |
| SUMO1 | 2.119475 | 0.052152 |
| DMAP1 | 2.115848 | 0.14984 |
| SPEN | 2.082621 | 0.04706 |
| PRKDC | 2.054387 | 0.020214 |
| KIR2DL4 | 2.022482 | 0.168389 |
| APC | 2.020118 | 0.014562 |
| MEF2D | 2.018638 | 0.205228 |
| TRRAP | 1.985487 | 0.034685 |
| BAZ2A | 1.963241 | 0.057413 |
| SUV39H1 | 1.958921 | 0.343539 |
| GTF3C4 | 1.956398 | 0.011402 |
| PADI4 | 1.936776 | 0.110304 |
| ZNF148 | 1.926952 | 0.101175 |
| NCOA2 | 1.914404 | 0.13113 |
| BAZ2B | 1.912741 | 0.213506 |
| KAT5 | 1.899023 | 0.042116 |
| GSTP1 | 1.872775 | 0.103465 |
| C20orf20 | 1.841951 | 0.132104 |
| SMAD3 | 1.835045 | 0.000715 |
| NCOA1 | 1.822696 | 0.033774 |
| CDY1B | 1.811376 | 0.66951 |
| PHF16 | 1.791378 | 0.032315 |
| MLH1 | 1.774602 | 0.041801 |
| NR2C1 | 1.763209 | 0.143539 |
| CDKN1A | 1.75747 | 0.009267 |
| GABPA | 1.745357 | 0.26862 |
| GMNN | 1.744971 | 0.106884 |
| GSG2 | 1.743216 | 0.094324 |
| ATAD2 | 1.737997 | 0.014856 |
| IFI16 | 1.731342 | 0.037746 |
| CCND1 | 1.728521 | 0.03016 |
| PLA2G16 | 1.710823 | 0.064799 |
| TWIST1 | 1.70272 | 0.360724 |
| MBD2 | 1.693068 | 0.20412 |
| IFNB1 | 1.686817 | 0.117278 |
| CHD3 | 1.685103 | 0.025845 |
| SPI1 | 1.680621 | 0.19609 |
| HIF1A | 1.669651 | 0.088802 |
| RCOR1 | 1.667394 | 0.113233 |
| RUNX1T1 | 1.66331 | 0.22652 |
| CFLAR | 1.663004 | 0.100039 |
| CDY2B | 1.632484 | 0.246241 |
| TFCP2 | 1.620896 | 0.066323 |
| ZMYM2 | 1.600876 | 0.048111 |
| CDY1 | 1.595909 | 0.034201 |
| MAPK8 | 1.595626 | 0.105843 |
| E2F1 | 1.581609 | 0.317282 |
| IDH2 | 1.571751 | 0.224545 |
| CCNB1 | 1.558334 | 0.149589 |
| CLGN | 1.534736 | 0.19762 |
| TP53 | 1.528972 | 0.061943 |
| TARDBP | 1.527738 | 0.074554 |
| KAT2B | 1.522754 | 0.055204 |
| RERE | 1.517957 | 0.036769 |
| HDAC5 | 1.514749 | 0.052821 |
| TGFB1 | 1.512247 | 0.009211 |
| MKI67 | 1.509564 | 0.279975 |
| SP2 | 1.507101 | 0.26521 |
| WDR82 | 1.50316 | 0.068023 |
| H2AFZ | 1.493633 | 0.029669 |
| FOXP3 | 1.487303 | 0.358275 |
| KRT23 | 1.484133 | 0.092025 |
| MICB | 1.476221 | 0.083149 |
| CCDC101 | 1.471987 | 0.192941 |
| RFC1 | 1.470751 | 0.107359 |
| RAP1GAP | 1.47004 | 0.055147 |
| EGFR | 1.466418 | 0.160559 |
| HDAC8 | 1.459179 | 0.070388 |
| NIPBL | 1.458736 | 0.019252 |
| LATS1 | 1.45133 | 0.02422 |
| MLL | 1.446532 | 0.161901 |
| TGFBR1 | 1.446509 | 0.2189 |
| HNRNPU | 1.444872 | 0.195762 |
| PKN2 | 1.443438 | 0.312938 |
| BCAS3 | 1.442768 | 0.115198 |
| TERT | 1.441849 | 0.060187 |
| BRMS1 | 1.437768 | 0.019876 |
| CDY2A | 1.436019 | 0.380472 |
| NCOR2 | 1.430601 | 0.027334 |
| SATB2 | 1.428661 | 0.052337 |
| SALL1 | 1.428215 | 0.042382 |
| SETD1B | 1.418378 | 0.352216 |
| RARA | 1.409808 | 0.511328 |
| MEF2C | 1.408539 | 0.342254 |
| CDK1 | 1.406172 | 0.049133 |
| BCL11B | 1.396615 | 0.106804 |
| YEATS2 | 1.392736 | 0.000645 |
| CHD1 | 1.385509 | 0.054394 |
| ELP4 | 1.381654 | 0.012584 |
| NOC2L | 1.381621 | 0.005973 |
| MLL3 | 1.381313 | 0.069742 |
| BRPF3 | 1.378898 | 0.167998 |
| NFKB1 | 1.360332 | 0.069843 |
| CASP8 | 1.359734 | 0.021175 |
| PEA15 | 1.3581 | 0.077961 |
| CD4 | 1.357769 | 0.123564 |
| CBX5 | 1.356671 | 0.093802 |
| USF1 | 1.356343 | 0.423359 |
| MAP2K4 | 1.356072 | 0.033336 |
| NFKB2 | 1.352113 | 0.117321 |
| EP300 | 1.351336 | 0.008618 |
| HDAC2 | 1.34946 | 0.244825 |
| IFNG | 1.340469 | 0.214563 |
| RUVBL2 | 1.337488 | 0.148837 |
| RHOB | 1.336061 | 0.186296 |
| MEF2A | 1.325135 | 0.058873 |
| CTR9 | 1.324755 | 0.120487 |
| NCOA3 | 1.316417 | 0.09717 |
| IL5 | 1.31449 | 0.074414 |
| BRCA2 | 1.307471 | 0.146177 |
| HNF4A | 1.303522 | 0.083443 |
| DDX53 | 1.300401 | 0.058196 |
| EED | 1.294184 | 0.290409 |
| IL24 | 1.284432 | 0.336421 |
| PROM1 | 1.281521 | 0.135175 |
| VEGFA | 1.275085 | 0.578396 |
| SUPT3H | 1.273981 | 0.109976 |
| ATXN7 | 1.266507 | 0.320093 |
| DNMT1 | 1.265959 | 0.02695 |
| SDC1 | 1.263003 | 0.079211 |
| TAF7 | 1.261491 | 0.130196 |
| KDM4A | 1.259146 | 0.613633 |
| AES | 1.256752 | 0.192033 |
| NUDT21 | 1.249718 | 0.058988 |
| NFYB | 1.249438 | 0.130802 |
| UHRF1BP1 | 1.246025 | 0.121585 |
| BRD1 | 1.236461 | 0.058755 |
| RUVBL1 | 1.235495 | 0.124098 |
| SMAD7 | 1.232964 | 0.12095 |
| BAZ1B | 1.232312 | 0.045874 |
| RBPJ | 1.231345 | 0.033005 |
| MTA2 | 1.230373 | 0.045221 |
| SUPT7L | 1.230167 | 0.060147 |
| ZMYM3 | 1.228456 | 0.113153 |
| MAGEA2 | 1.227242 | 0.030533 |
| CDK5R1 | 1.224767 | 0.449125 |
| MLL2 | 1.223347 | 0.207863 |
| RB1 | 1.220636 | 0.027249 |
| PHF21A | 1.219026 | 0.041777 |
| TAF6L | 1.216089 | 0.071181 |
| SMYD2 | 1.213695 | 0.057812 |
| CLOCK | 1.208267 | 0.065789 |
| SETDB2 | 1.206353 | 0.029933 |
| PML | 1.200961 | 0.071679 |
| PRDM1 | 1.199998 | 0.031529 |
| DPY30 | 1.19371 | 0.059896 |
| MGMT | 1.189418 | 0.050988 |
| MYC | 1.185714 | 0.215821 |
| SALL3 | 1.185291 | 0.085353 |
| IL13 | 1.168584 | 0.141312 |
| CXXC1 | 1.162825 | 0.100761 |
| TOP2B | 1.160575 | 0.164227 |
| ZNF217 | 1.156789 | 0.113157 |
| KLF14 | 1.156194 | 0.006712 |
| NKX3-1 | 1.153988 | 0.02949 |
| TRIM29 | 1.152696 | 0.001042 |
| KDM1A | 1.148962 | 0.210084 |
| RECK | 1.145797 | 0.037568 |
| SETD3 | 1.145261 | 0.285076 |
| CSRP2BP | 1.138262 | 0.100684 |
| SETD1A | 1.136995 | 0.134904 |
| USP22 | 1.136202 | 0.114927 |
| ING2 | 1.134242 | 0.182222 |
| KAT6A | 1.13154 | 0.402474 |
| C16orf53 | 1.128591 | 0.149928 |
| TADA1 | 1.126079 | 0.364332 |
| SUZ12 | 1.119057 | 1.223702 |
| N6AMT1 | 1.1181 | 0.146334 |
| KPNA2 | 1.117656 | 0.217184 |
| PEX14 | 1.117068 | 0.255198 |
| ARID1A | 1.115998 | 0.078403 |
| MBD3 | 1.113038 | 0.105622 |
| SF3B3 | 1.108815 | 0.08127 |
| SIN3B | 1.105758 | 0.054678 |
| NKAP | 1.10296 | 0.071532 |
| CAMTA2 | 1.099637 | 0.111942 |
| F3 | 1.094147 | 0.094744 |
| CIR1 | 1.088826 | 0.085504 |
| CECR2 | 1.077054 | 0.064017 |
| ANKRD1 | 1.076987 | 0.168455 |
| NFE2L2 | 1.075071 | 0.228188 |
| NR3C1 | 1.07376 | 0.223865 |
| TUBA8 | 1.073504 | 0.01955 |
| MTF1 | 1.070018 | 0.222578 |
| EHMT2 | 1.068657 | 0.004415 |
| FGFR3 | 1.066121 | 0.004651 |
| MECOM | 1.062529 | 0.019563 |
| CREBBP | 1.057707 | 0.164042 |
| BRCA1 | 1.055937 | 0.097398 |
| DAXX | 1.052737 | 0.001142 |
| ABCB1 | 1.052273 | 0.146848 |
| NSD1 | 1.047448 | 0.125504 |
| MIER1 | 1.043516 | 0.036453 |
| BHLHE41 | 1.042951 | 0.164137 |
| CDC73 | 1.039137 | 0.181696 |
| TNFRSF9 | 1.039129 | 0.058632 |
| PAX6 | 1.037401 | 0.468393 |
| KAT7 | 1.037004 | 0.123777 |
| VDR | 1.033722 | 0.060818 |
| PRKAB1 | 1.033714 | 0.117138 |
| DCC | 1.03253 | 0.178887 |
| PRDX1 | 1.030073 | 0.010926 |
| CITED2 | 1.029938 | 0.315317 |
| NCOR1 | 1.027946 | 0.163666 |
| ESR1 | 1.027744 | 0.25989 |
| KRAS | 1.027201 | 0.003579 |
| HDAC6 | 1.024242 | 0.234 |
| AURKB | 1.023532 | 0.074672 |
| UHRF1 | 1.021851 | 0.190562 |
| MSH2 | 1.016581 | 0.039809 |
| TADA2B | 1.016425 | 0.044812 |
| MLL5 | 1.015729 | 0.047391 |
| CDK2 | 1.009869 | 0.096811 |
| MORF4L1 | 1.008464 | 0.213184 |
| KAT6B | 1.008352 | 0.020562 |
| BIRC5 | 1.003109 | 0.047286 |
| FES | 1.002961 | 0.06576 |
| CDYL | 1.00149 | 0.132779 |
| HRAS | 1.000696 | 0.154032 |
| CIITA | 0.999334 | 0.045764 |
| SENP1 | 0.99401 | 0.04888 |
| PRDM14 | 0.992507 | 0.00532 |
| RBBP4 | 0.990023 | 0.041874 |
| SUN1 | 0.988567 | 0.067196 |
| PKN1 | 0.985086 | 0.122444 |
| RELN | 0.985057 | 0.134355 |
| BRPF1 | 0.981796 | 0.080468 |
| TAF10 | 0.97957 | 0.192907 |
| CSNK2A1 | 0.978534 | 0.101121 |
| TAF5L | 0.975694 | 0.257281 |
| SUV39H2 | 0.971945 | 0.072917 |
| TRAF6 | 0.971874 | 0.036465 |
| SPOP | 0.971624 | 0.221885 |
| MYB | 0.970653 | 0.020559 |
| MMP9 | 0.97026 | 0.032324 |
| TUBA1B | 0.961907 | 0.158951 |
| SETD8 | 0.961421 | 0.090324 |
| NAT10 | 0.953699 | 0.085111 |
| DIP2B | 0.949158 | 0.117463 |
| HMG20B | 0.946187 | 0.038692 |
| EZH1 | 0.94278 | 0.059411 |
| PRMT7 | 0.942367 | 0.092663 |
| BRMS1L | 0.941621 | 0.00168 |
| TOP2A | 0.941452 | 0.029488 |
| MED24 | 0.939427 | 0.064759 |
| HNF1A | 0.939188 | 0.042303 |
| TUBA3C | 0.934268 | 0.074515 |
| PRMT1 | 0.932275 | 0.139063 |
| HPGD | 0.927544 | 0.047463 |
| MYOCD | 0.926614 | 0.130867 |
| PRMT2 | 0.925298 | 0.066321 |
| SAP18 | 0.92247 | 0.099177 |
| JARID2 | 0.919535 | 0.202206 |
| SUV420H1 | 0.918594 | 0.173778 |
| CXADR | 0.916546 | 0.053083 |
| PRF1 | 0.904105 | 0.011029 |
| SLC2A4 | 0.904059 | 0.009424 |
| DOT1L | 0.902864 | 0.16045 |
| TNFSF10 | 0.901029 | 0.18135 |
| EDF1 | 0.900237 | 0.014248 |
| SUV420H2 | 0.897968 | 0.172116 |
| LEO1 | 0.896723 | 0.050959 |
| PTGS2 | 0.894102 | 0.497395 |
| MIF | 0.893397 | 0.194554 |
| AR | 0.886335 | 0.078388 |
| H2AFX | 0.883926 | 0.422759 |
| NQO1 | 0.883865 | 0.111466 |
| NFYA | 0.883787 | 0.137681 |
| RUNX2 | 0.883346 | 0.174298 |
| SETDB1 | 0.882963 | 0.062165 |
| PHF20 | 0.88212 | 0.121558 |
| HINFP | 0.881388 | 0.051187 |
| JUN | 0.8802 | 0.059006 |
| EPAS1 | 0.879998 | 0.033154 |
| ARRB1 | 0.878782 | 0.009635 |
| TGFBR2 | 0.875804 | 0.019383 |
| PDK4 | 0.874145 | 0.012672 |
| YWHAB | 0.873961 | 0.082014 |
| RUNX1 | 0.873116 | 0.011271 |
| ABCC1 | 0.87122 | 0.09479 |
| CDK11A | 0.870977 | 0.003716 |
| SIRT2 | 0.859159 | 0.02776 |
| PHB | 0.857689 | 0.119633 |
| DNMT3L | 0.856446 | 0.116672 |
| SUDS3 | 0.856007 | 0.031787 |
| ING4 | 0.854957 | 0.14643 |
| PRMT6 | 0.85259 | 0.015261 |
| TCF21 | 0.849189 | 0.095611 |
| RBBP7 | 0.848903 | 0.079747 |
| CXCL12 | 0.84876 | 0.079965 |
| TUBA4A | 0.844379 | 0.279532 |
| HIC2 | 0.844321 | 0.025511 |
| HPSE | 0.843136 | 0.160091 |
| WHSC1 | 0.842313 | 0.122163 |
| CXCR4 | 0.840796 | 0.058992 |
| PRKCA | 0.839601 | 0.066119 |
| E2F6 | 0.838928 | 0.065776 |
| CHRNE | 0.836508 | 0.038432 |
| STAT3 | 0.835259 | 0.017378 |
| HEY2 | 0.825149 | 0.023005 |
| NFYC | 0.822831 | 0.015783 |
| EZH2 | 0.821495 | 0.085455 |
| TAF12 | 0.820841 | 0.053659 |
| TPPP | 0.820309 | 0.008001 |
| BCOR | 0.818979 | 0.006866 |
| SP1 | 0.818448 | 0.09332 |
| PAXIP1 | 0.818335 | 0.047976 |
| ULBP2 | 0.813605 | 0.099239 |
| MLL4 | 0.813084 | 0.148387 |
| MAGEA1 | 0.811527 | 0.118212 |
| TBL1XR1 | 0.810227 | 0.193963 |
| RELA | 0.80782 | 0.08096 |
| DEK | 0.806225 | 0.088955 |
| AKT1 | 0.805037 | 0.496321 |
| WNK4 | 0.802826 | 0.060557 |
| ETV6 | 0.797355 | 0.050518 |
| BMI1 | 0.795599 | 0.236267 |
| NRIP1 | 0.79462 | 0.179617 |
| ANKRA2 | 0.791872 | 0.004883 |
| SOD2 | 0.791698 | 0.164835 |
| GATA3 | 0.790259 | 0.1663 |
| TAL1 | 0.787454 | 0.141897 |
| N6AMT2 | 0.786784 | 0.106426 |
| PHF17 | 0.785948 | 0.206761 |
| NFATC1 | 0.781722 | 0.116544 |
| TFAP4 | 0.780982 | 0.038572 |
| GADD45A | 0.780126 | 0.145771 |
| TUBA1A | 0.777987 | 0.070961 |
| HIPK2 | 0.777929 | 0.270004 |
| ELP3 | 0.771886 | 0.025216 |
| HAT1 | 0.768189 | 0.056701 |
| ING5 | 0.76759 | 0.133088 |
| TH | 0.765825 | 0.161472 |
| POU2F1 | 0.765584 | 0.056692 |
| HR | 0.764332 | 0.055399 |
| LOXL1 | 0.763227 | 0.091752 |
| BAZ1A | 0.763213 | 0.083356 |
| CYP7A1 | 0.760439 | 0.093737 |
| HDAC10 | 0.759193 | 0.076176 |
| DUSP4 | 0.757087 | 0.10024 |
| SNAI1 | 0.752073 | 0.159888 |
| GPS2 | 0.751587 | 0.070912 |
| BRD8 | 0.750801 | 0.03568 |
| SRCAP | 0.750639 | 0.032659 |
| CD70 | 0.747283 | 0.029157 |
| GCM1 | 0.742235 | 0.04318 |
| BANP | 0.741337 | 0.004149 |
| GLI3 | 0.739888 | 0.021878 |
| ASH2L | 0.738826 | 0.107893 |
| RUNX3 | 0.738624 | 0.028648 |
| PAF1 | 0.738335 | 0.119545 |
| AMD1 | 0.733483 | 0.175237 |
| CYBB | 0.728539 | 0.045344 |
| CSK | 0.727852 | 0.044279 |
| HDAC1 | 0.726997 | 0.062123 |
| SIRT1 | 0.725727 | 0.112278 |
| HDAC7 | 0.724565 | 0.147498 |
| SETD2 | 0.721119 | 0.024466 |
| TUBA1C | 0.717963 | 0.198771 |
| HDAC3 | 0.717661 | 0.038437 |
| MCRS1 | 0.713804 | 0.140658 |
| EP400 | 0.713145 | 0.070764 |
| PAEP | 0.710022 | 0.066282 |
| PYGO2 | 0.706929 | 0.028742 |
| CD7 | 0.706233 | 0.045954 |
| PPARG | 0.702346 | 0.05323 |
| OGT | 0.692176 | 0.039326 |
| TAF5 | 0.690125 | 0.067591 |
| PTPN6 | 0.68906 | 0.066074 |
| DNMT3B | 0.683883 | 0.044812 |
| HDAC9 | 0.68266 | 0.047391 |
| VIM | 0.682407 | 0.096811 |
| ATM | 0.679982 | 0.013184 |
| PRMT5 | 0.675565 | 0.020562 |
| DSG1 | 0.67532 | 0.047286 |
| ING3 | 0.672389 | 0.06576 |
| CASP2 | 0.669253 | 0.132779 |
| PWWP2A | 0.669005 | 0.154032 |
| EHMT1 | 0.666011 | 0.045764 |
| YWHAE | 0.663513 | 0.04888 |
| ZBTB7A | 0.659107 | 0.00532 |
| C10orf90 | 0.65765 | 0.041874 |
| TADA2A | 0.648606 | 0.067196 |
| SAP30 | 0.646996 | 0.122444 |
| AURKA | 0.643001 | 0.134355 |
| BPTF | 0.63983 | 0.080468 |
| ZBTB16 | 0.634699 | 0.192907 |
| PHF15 | 0.63377 | 0.101121 |
| HDAC4 | 0.632405 | 0.257281 |
| NSUN5 | 0.631909 | 0.072917 |
| RAD9A | 0.631834 | 0.036465 |
| DNMT3A | 0.628056 | 0.221885 |
| ORC1 | 0.62 | 0.020559 |
| SP3 | 0.617935 | 0.032324 |
| EPC1 | 0.61629 | 0.158951 |
| SMYD3 | 0.612742 | 0.090324 |
| MEN1 | 0.596165 | 0.085111 |
| PCNA | 0.595327 | 0.117463 |
| CARM1 | 0.594609 | 0.038692 |
| MBD5 | 0.59023 | 0.059411 |
| CTNNB1 | 0.589965 | 0.092663 |
| TAF1 | 0.588343 | 0.00168 |
| HDAC11 | 0.588213 | 0.029488 |
| EGR1 | 0.588046 | 0.064759 |
| CDKN2A | 0.586904 | 0.042303 |
| HES1 | 0.58637 | 0.074515 |
| RFXANK | 0.58438 | 0.139063 |
| HDLBP | 0.582397 | 0.047463 |
| ARID4B | 0.582345 | 0.130867 |
| HBB | 0.573392 | 0.066321 |
| TAF1L | 0.571685 | 0.099177 |
| TRIM68 | 0.569824 | 0.202206 |
| SKI | 0.55455 | 0.173778 |
| MTA1 | 0.547317 | 0.053083 |
| HMGA2 | 0.545919 | 0.011029 |
| ATG7 | 0.545569 | 0.009424 |
| ACTB | 0.540832 | 0.16045 |
| MAPT | 0.537573 | 0.18135 |
| HOXA10 | 0.535626 | 0.014248 |
| EFCAB6 | 0.533825 | 0.172116 |
| HIST4H4 | 0.526152 | 0.050959 |
| STAT1 | 0.525319 | 0.497395 |
| WDR5 | 0.52448 | 0.194554 |
| CTAG1B | 0.512581 | 0.078388 |
| PWWP2B | 0.510513 | 0.422759 |
| PRKCB | 0.509845 | 0.111466 |
| MGEA5 | 0.500946 | 0.137681 |
| CTBP1 | 0.496068 | 0.174298 |
| TRDMT1 | 0.491156 | 0.062165 |
| KIAA1310 | 0.482535 | 0.121558 |
| CBX4 | 0.469116 | 0.051187 |
| KIAA1267 | 0.461575 | 0.059006 |
| CBFA2T2 | 0.460239 | 0.033154 |
| ARID4A | 0.419073 | 0.009635 |
| CHEK1 | 0.416027 | 0.019383 |
| SIN3A | 0.404453 | 0.012672 |
| YY1 | 0.394037 | 0.082014 |
| SIRT6 | 0.389797 | 0.011271 |
| SAP130 | 0.345884 | 0.09479 |
| SETD7 | 0.345217 | 0.003716 |
| SAP30L | 0.332915 | 0.02776 |
| ATF2 | 0.315087 | 0.119633 |
| KAT8 | 0.290235 | 0.116672 |
| RBBP5 | 0.287091 | 0.031787 |
| C12orf41 | 0.286897 | 0.14643 |
| HCFC1 | 0.274921 | 0.015261 |
| TBL1X | 0.236665 | 0.095611 |
| CTCF | 0.219316 | 0.079747 |

Gene: Target of an siRNA,
Fold Change: Fold change in FXN mRNA expression compared to control,
STDEV: standard deviation

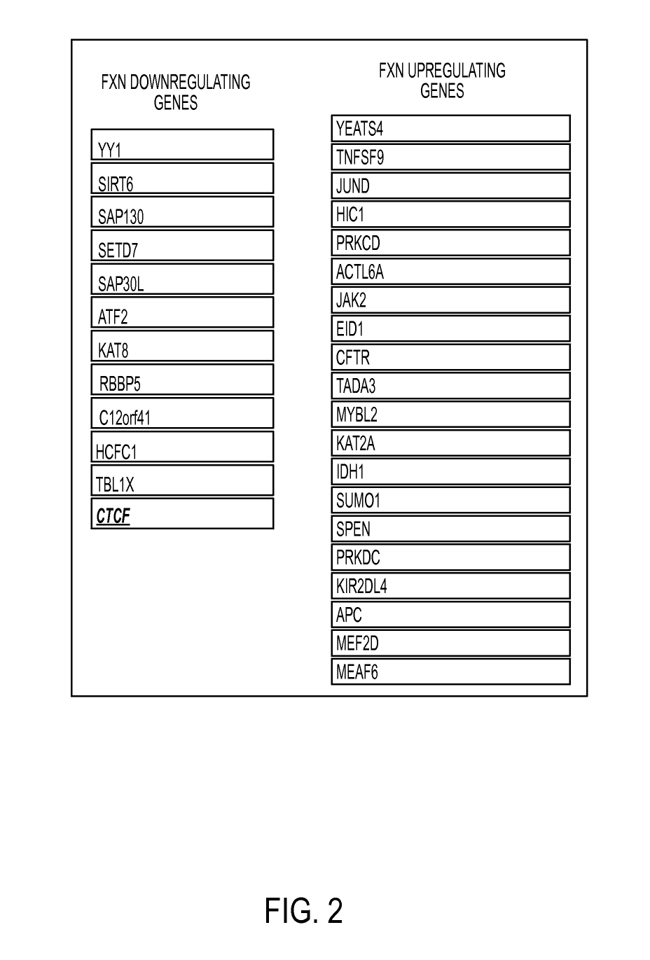

FIG. 2 depicts a list of the genes that upon knockdown downregulated or upregulated FXN mRNA at least two-fold. These genes were analyzed using the Database for Annotation, Visualization and Integrated Discovery (DAVID) Functional annotation tool to identify pathways that were enriched in the FXN upregulating and downregulating gene sets. Tables 7 and 8 show the pathways identified.

TABLE 7
Pathways identified using the FXN upregulating gene set

| Category | Term | Count | % | PValue | Genes |
|---|---|---|---|---|---|
| GOTERM_BP_FAT | GO: 0043968~histone H2A | 4 | 18.181818 | 0.017418 | MEAF6, YEATS4, ACTL6A, |
| | acetylation | | | | DMAP1 |
| GOTERM_CC_FAT | GO: 0035267~NuA4 | 4 | 18.181818 | 0.027984 | MEAF6, YEATS4, ACTL6A, |
| | histone acetyltransferase | | | | DMAP1 |
| | complex | | | | |
| GOTERM_BP_FAT | GO: 0006473~protein | 6 | 27.272727 | 0.031248 | KAT2A, MEAF6, YEATS4, |
| | amino acid acetylation | | | | TADA3, ACTL6A, DMAP1 |
| GOTERM_BP_FAT | GO: 0016573~histone | 6 | 27.272727 | 0.031248 | KAT2A, MEAF6, YEATS4, |
| | acetylation | | | | TADA3, ACTL6A, DMAP1 |
| GOTERM_BP_FAT | GO: 0043543~protein | 6 | 27.272727 | 0.031248 | KAT2A, MEAF6, YEATS4, |
| | amino acid acylation | | | | TADA3, ACTL6A, DMAP1 |
| GOTERM_CC_FAT | GO: 0043189~H4/H2A | 4 | 18.181818 | 0.033873 | MEAF6, YEATS4, ACTL6A, |
| | histone acetyltransferase | | | | DMAP1 |
| | complex | | | | |
| GOTERM_MF_FAT | GO: 0000166~nucleotide | 8 | 36.363636 | 0.037148 | MEF2D, PRKDC, IDH1, |
| | binding | | | | ACTL6A, JAK2, CFTR, SPEN, |
| | | | | | PRKCD |
| GOTERM_BP_FAT | GO: 0043967~histone H4 | 4 | 18.181818 | 0.038931 | MEAF6, YEATS4, ACTL6A, |
| | acetylation | | | | DMAP1 |
| GOTERM_CC_FAT | GO: 0000123~histone | 6 | 27.272727 | 0.039436 | KAT2A, MEAF6, YEATS4, |
| | acetyltransferase | | | | TADA3, ACTL6A, DMAP1 |
| | complex | | | | |
| GOTERM_MF_FAT | GO: 0043560~insulin | 2 | 9.0909091 | 0.0815 | JAK2, PRKCD |
| | receptor substrate | | | | |
| | binding | | | | |
| SP_PIR_KEYWORDS | membrane | 6 | 27.272727 | 0.089156 | SUMO1, JAK2, CFTR, |
| | | | | | TNFSF9, PRKCD, KIR2DL4 |

Category: Database/resource where the terms orient;
Term: Enriched terms associated with the gene set;
Count: Number of genes involved in the term;
%: Percentage of the genes involved out of the total gene set;
PValue: Modified Fisher Exact P-value, EASE Score;
Genes: Genes involved in the term

TABLE 8
Pathways identified using the FXN downregulating gene set

| Category | Term | Count | % | PValue | Genes |
|---|---|---|---|---|---|
| GOTERM_MF_FAT | GO: 0003712~transcription | 9 | 40.90909091 | 0.006557335 | SIN3A, SAP130, YY1, CBX4, |
| | cofactor activity | | | | HCFC1, CTCF, TBL1X, |
| | | | | | CBFA2T2, ATF2 |
| GOTERM_MF_FAT | GO: 0003714~transcription | 6 | 27.27272727 | 0.009655309 | SIN3A, YY1, CBX4, CTCF, |
| | corepressor activity | | | | TBL1X, CBFA2T2 |
| GOTERM_MF_FAT | GO: 0008134~transcription | 10 | 45.45454545 | 0.013184213 | CTBP1, SIN3A, SAP130, YY1, |
| | factor binding | | | | CBX4, HCFC1, CTCF, TBL1X, |
| | | | | | CBFA2T2, ATF2 |
| GOTERM_MF_FAT | GO: 0016564~transcription | 8 | 36.36363636 | 0.018617445 | CTBP1, SIN3A, ARID4A, YY1, |
| | repressor activity | | | | CBX4, CTCF, TBL1X, |
| | | | | | CBFA2T2 |
| GOTERM_BP_FAT | GO: 0031327~negative | 9 | 40.90909091 | 0.046413359 | CTBP1, SIN3A, ARID4A, |
| | regulation of cellular | | | | MGEA5, CBX4, SIRT6, CTCF, |
| | biosynthetic process | | | | TBL1X, CBFA2T2 |
| GOTERM_BP_FAT | GO: 0009890~negative | 9 | 40.90909091 | 0.046413359 | CTBP1, SIN3A, ARID4A, |
| | regulation of biosynthetic | | | | MGEA5, CBX4, SIRT6, CTCF, |
| | process | | | | TBL1X, CBFA2T2 |
| GOTERM_BP_FAT | GO: 0010558~negative | 9 | 40.90909091 | 0.046413359 | CTBP1, SIN3A, ARID4A, |
| | regulation of | | | | MGEA5, CBX4, SIRT6, CTCF, |
| | macromolecule | | | | TBL1X, CBFA2T2 |
| | biosynthetic process | | | | |
| GOTERM_BP_FAT | GO: 0051253~negative | 8 | 36.36363636 | 0.047407616 | CTBP1, SIN3A, ARID4A, |
| | regulation of RNA | | | | CBX4, SIRT6, CTCF, TBL1X, |
| | metabolic process | | | | CBFA2T2 |
| GOTERM_BP_FAT | GO: 0045892~negative | 8 | 36.36363636 | 0.047407616 | CTBP1, SIN3A, ARID4A, |
| | regulation of | | | | CBX4, SIRT6, CTCF, TBL1X, |
| | transcription, DNA- | | | | CBFA2T2 |
| | dependent | | | | |
| GOTERM_CC_FAT | GO: 0005694~chromosome | 7 | 31.81818182 | 0.060583982 | SIN3A, ARID4A, CBX4, |
| | | | | | SETD7, SIRT6, CHEK1, CTCF |
| GOTERM_BP_FAT | GO: 0010605~negative | 9 | 40.90909091 | 0.074797395 | CTBP1, SIN3A, ARID4A, |
| | regulation of | | | | MGEA5, CBX4, SIRT6, CTCF, |
| | macromolecule metabolic | | | | TBL1X, CBFA2T2 |
| | process | | | | |
| SP_PIR_KEYWORDS | phosphoprotein | 19 | 86.36363636 | 0.097277404 | CTBP1, RBBP5, ARID4A, YY1, |
| | | | | | CBX4, HCFC1, SIRT6, CHEK1, |
| | | | | | CTCF, CBFA2T2, C12ORF41, |
| | | | | | ATF2, PRKCB, KIAA1310, |
| | | | | | SIN3A, KIAA1267, SAP130, |
| | | | | | MGEA5, SAP30L |
| GOTERM_BP_FAT | GO: 0016481~negative | 8 | 36.36363636 | 0.099303629 | CTBP1, SIN3A, ARID4A, |
| | regulation of | | | | CBX4, SIRT6, CTCF, TBL1X, |
| | transcription | | | | CBFA2T2 |

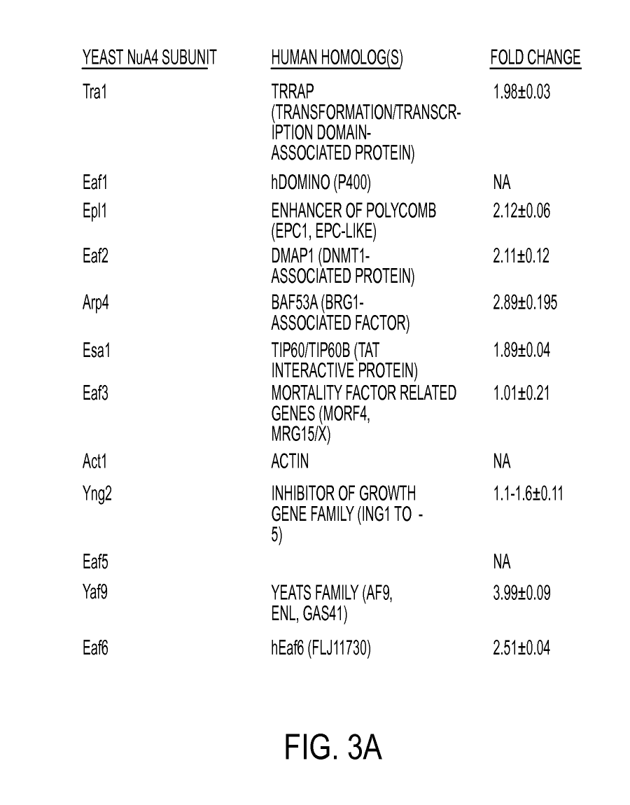

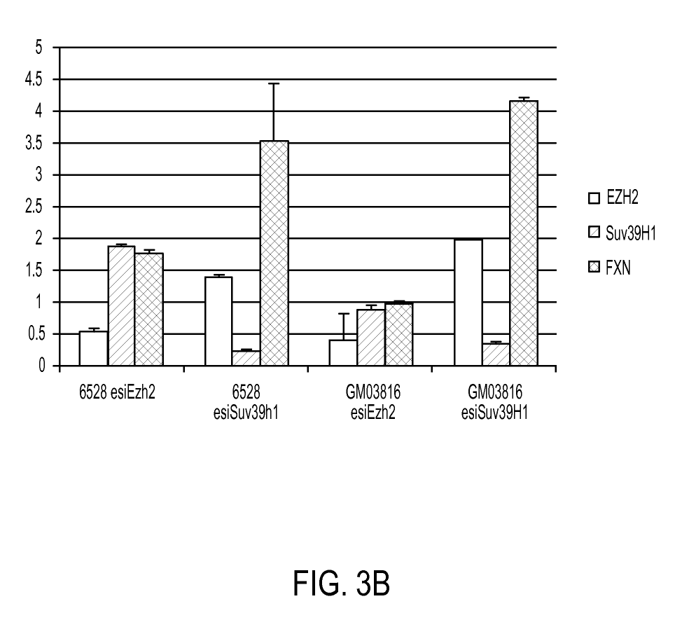

Knockdown of the YEATS gene was found to upregulate FXN to the greatest extent under the conditions evaluated. YEATS is known to be a component of the NuA4 Histone Acetyltransferase complex, which was identified as an enriched pathway by DAVID analysis. The siRNA results for other components of the NuA4 Histone Acetyltransferase complex were examined to see if knockdown of other NuA4 Histone Acetyltransferase complex components also resulted in upregulation of FXN mRNA. FIG. 3A shows that knockdown of several of the components of the NuA4 Histone Acetyltransferase complex caused upregulation of FXN mRNA. These results suggest that the NuA4 Histone Acetyltransferase complex is involved in downregulating FXN expression, and that knockdown of one or more components of the complex may result in FXN mRNA upregulation.

Knockdown of the histone-lysine N-methyltransferase SUV39H1 was also found to upregulate FXN mRNA levels (Table 6, FIG. 3B). These results suggest that histone-lysine N-methyltransferases, such as SUV39H1, are also involved in downregulating FXN expression and that knockdown of one or more histone-lysine N-methyltransferases may result in FXN mRNA upregulation.

### Example 2

### Validation of siRNA Hits in a Second Cell Line

The same siRNA pool was tested in a second FRDA cell line (GM04078) using the same methods as described in Example 1. The summary of the data is provided in Table 9. The correlation of fold change of FXN mRNA for each siRNA target between the first and second cell lines was very high (0.85) and all the top upregulating/downregulating responders for FXN mRNA were 100% reproducible in both lines. These results indicate that the negative epigenetic regulators of FXN identified in Example 1 are capable of regulating FXN levels in multiple cell lines.

TABLE 9
siRNA evaluation results in GM04078 FRDA cell line

| | Fold | |
|---|---|---|
| Gene | Change | STDEV |
| TNFSF9 | 5.910628 | 1.442592 |
| HIC1 | 3.79222 | 0.509594 |
| ACTL6A | 3.649537 | 0.918474 |
| TADA3 | 3.63286 | 1.324446 |
| JUND | 3.424003 | 0.721256 |
| MEF2D | 3.170798 | 0.226705 |
| DMAP1 | 3.168941 | 0.235867 |
| PRKCD | 3.085134 | 0.093737 |
| YEATS4 | 2.9974 | 0.785606 |
| MYBL2 | 2.982222 | 0.582387 |
| CFLAR | 2.848965 | 0.537174 |
| SUMO1 | 2.671226 | 0.006546 |
| TWIST1 | 2.669304 | 0.169964 |
| KAT2A | 2.646844 | 0.505434 |
| CFTR | 2.605991 | 0.125125 |
| TP53 | 2.587413 | 0.01395 |
| IFNB1 | 2.572815 | 0.434231 |
| RAP1GAP | 2.555712 | 0.063877 |
| MEF2C | 2.547859 | 0.546463 |
| SMAD3 | 2.534138 | 0.312204 |
| CDK1 | 2.51694 | 0.108525 |
| HNF1A | 2.48174 | 0.706655 |
| HNRNPU | 2.477997 | 0.34863 |
| SALL1 | 2.445191 | 0.611896 |
| NFKB1 | 2.414303 | 0.449406 |
| SATB2 | 2.387531 | 0.017553 |
| MAPK8 | 2.358503 | 0.443562 |
| IDH1 | 2.314138 | 0.171112 |
| USF1 | 2.281319 | 0.203232 |
| FOXP3 | 2.251071 | 0.577201 |
| RERE | 2.235115 | 0.108411 |
| BCL11B | 2.228773 | 0.433105 |
| SUV39H1 | 2.213635 | 0.703845 |
| C20orf20 | 2.18294 | 0.173146 |
| NIPBL | 2.164491 | 0.019096 |
| CCND1 | 2.163809 | 0.030755 |
| SPEN | 2.158165 | 0.190154 |
| RARA | 2.132107 | 0.245035 |
| ATXN7 | 2.111207 | 0.028972 |
| CIR1 | 2.107452 | 0.001033 |
| BAZ2A | 2.105362 | 0.083562 |
| CHD1 | 2.104623 | 0.177214 |
| PEA15 | 2.104046 | 0.456311 |
| TADA1 | 2.079549 | 1.081316 |
| IFNG | 2.068108 | 0.504758 |
| YWHAE | 2.055071 | 0.920124 |
| NCOA2 | 2.045579 | 0.132251 |
| GMNN | 2.035796 | 0.188315 |
| ZMYM3 | 2.025216 | 0.282962 |
| H2AFZ | 2.021116 | 0.241134 |
| SPI1 | 2.014361 | 0.244248 |
| NCOA1 | 2.000822 | 0.18508 |
| CREBBP | 1.987823 | 0.666173 |
| RCOR1 | 1.986894 | 0.345896 |
| MEAF6 | 1.984914 | 0.31389 |
| CDY1 | 1.975774 | 0.300964 |
| KIR2DL4 | 1.972702 | 0.134292 |
| SMYD2 | 1.972411 | 0.218997 |
| RB1 | 1.971005 | 0.177512 |
| TCF21 | 1.970225 | 0.547094 |
| KRAS | 1.96191 | 0.414211 |
| GSTP1 | 1.960572 | 0.171787 |
| TERT | 1.945413 | 0.339613 |
| GSG2 | 1.94361 | 0.222426 |
| IFI16 | 1.936336 | 0.155478 |
| TRRAP | 1.935236 | 0.125117 |
| CDY1B | 1.928019 | 0.504413 |
| KAT5 | 1.924108 | 0.207072 |
| UHRF1BP1 | 1.898266 | 0.163547 |
| MBD2 | 1.896313 | 0.788364 |
| TAF7 | 1.894265 | 0.331599 |
| EP300 | 1.887058 | 0.33762 |
| NKAP | 1.880505 | 0.718167 |
| USP22 | 1.878313 | 0.404658 |
| MLL3 | 1.877268 | 0.289596 |
| PLA2G16 | 1.866196 | 0.031098 |
| SRCAP | 1.864067 | 0.435432 |
| CTR9 | 1.862841 | 0.006391 |
| RUVBL2 | 1.860471 | 0.242777 |
| MLL2 | 1.858536 | 0.403066 |
| SETDB2 | 1.846362 | 0.105822 |
| CDY2B | 1.840375 | 3.3E−15 |
| E2F1 | 1.834249 | 0.197402 |
| NFYB | 1.833774 | 0.542814 |
| ATAD2 | 1.8298 | 0.104873 |
| RFC1 | 1.828699 | 0.187891 |
| JAK2 | 1.827241 | 0.378771 |
| CDY2A | 1.823435 | 0.170451 |
| SP2 | 1.820392 | 0.082948 |
| HNF4A | 1.77444 | 0.780363 |
| HIF1A | 1.771572 | 0.549254 |
| MEF2A | 1.76059 | 0.022435 |
| ZNF217 | 1.758519 | 0.203813 |
| BAZ1B | 1.75718 | 0.958769 |
| PRKAB1 | 1.743587 | 0.022219 |
| TAF6L | 1.743031 | 0.473319 |
| BIRC5 | 1.729221 | 0.224809 |
| EED | 1.728013 | 0.184284 |
| IL24 | 1.724212 | 0.087851 |
| PADI4 | 1.724158 | 0.435553 |
| ELP4 | 1.718547 | 0.18495 |
| TAF12 | 1.716845 | 0.216524 |
| NCOR2 | 1.713332 | 0.339531 |
| MICB | 1.706158 | 0.485012 |
| PRDM1 | 1.697253 | 0.277432 |
| RBPJ | 1.696552 | 0.093915 |
| EPC2 | 1.685061 | 0.134478 |
| TFCP2 | 1.68223 | 0.103822 |
| SETD1A | 1.681001 | 0.119366 |
| BRMS1 | 1.673389 | 0.616959 |
| BCAS3 | 1.671848 | 0.137507 |
| KLF14 | 1.670757 | 0.004094 |
| MORF4L1 | 1.669224 | 0.458002 |
| IDH2 | 1.665195 | 0.038356 |
| CITED2 | 1.664975 | 0.062006 |
| MTA2 | 1.659185 | 0.122683 |
| ZMYM2 | 1.657127 | 0.349069 |
| PRMT2 | 1.652586 | 0.402557 |
| NR2C1 | 1.649787 | 0.189604 |
| DNMT1 | 1.642562 | 0.411042 |
| RUVBL1 | 1.642233 | 0.153513 |
| CDKN1A | 1.635584 | 0.14411 |
| CDYL | 1.631171 | 0.276091 |
| NSD1 | 1.629025 | 0.007984 |
| EID1 | 1.626959 | 0.446406 |
| HPGD | 1.626224 | 0.249293 |
| BRCA2 | 1.623737 | 0.266985 |
| SETD1B | 1.622057 | 0.230569 |
| BRCA1 | 1.616865 | 0.038035 |
| KAT2B | 1.615289 | 0.154147 |
| KRT23 | 1.593617 | 0.298943 |
| TARDBP | 1.593323 | 0.168367 |
| WDR82 | 1.590596 | 0.086493 |
| MKI67 | 1.587659 | 0.079339 |
| MLL | 1.572763 | 0.251758 |
| HDAC2 | 1.568287 | 0.168779 |
| FGFR3 | 1.564608 | 0.045238 |
| GTF3C4 | 1.559982 | 0.083301 |
| MLH1 | 1.558135 | 0.179829 |
| SALL3 | 1.557784 | 0.208577 |
| PHF16 | 1.554078 | 0.019804 |
| EGFR | 1.549861 | 0.277291 |
| BRPF3 | 1.549197 | 0.319648 |
| RECK | 1.546451 | 0.356814 |
| MSH2 | 1.541732 | 0.256483 |
| PTGS2 | 1.541399 | 0.320257 |
| BAZ2B | 1.541001 | 0.492843 |
| CD4 | 1.539739 | 0.088248 |
| HDAC8 | 1.534718 | 0.126978 |
| KAT6B | 1.524366 | 0.154392 |
| TOP2B | 1.519738 | 0.04543 |
| NCOA3 | 1.515915 | 0.234586 |
| ZNF148 | 1.515718 | 0.002972 |
| EHMT2 | 1.507618 | 0.10558 |
| CYP7A1 | 1.501413 | 0.106614 |
| SUV420H2 | 1.499707 | 0.218269 |
| PKN1 | 1.495558 | 0.315035 |
| CLGN | 1.489705 | 0.214646 |
| DCC | 1.489164 | 0.003649 |
| TAF5L | 1.484746 | 0.036382 |
| TNFRSF9 | 1.473452 | 0.034662 |
| HDAC5 | 1.470134 | 0.196127 |
| NAT10 | 1.462026 | 0.694932 |
| NOC2L | 1.451464 | 0.09384 |
| MAGEA2 | 1.448303 | 0.320333 |
| CBX5 | 1.43789 | 0.249626 |
| NUDT21 | 1.434775 | 0.203947 |
| PML | 1.425367 | 0.043307 |
| TFAP4 | 1.418233 | 0.454898 |
| GABPA | 1.416678 | 0.533298 |
| ING2 | 1.413519 | 0.040177 |
| SUV420H1 | 1.411008 | 0.082251 |
| PROM1 | 1.408753 | 0.071088 |
| DOT1L | 1.407563 | 0.033112 |
| NR3C1 | 1.407268 | 0.087541 |
| CASP8 | 1.405206 | 0.039253 |
| PRKCA | 1.404985 | 0.055076 |
| ING5 | 1.404806 | 0.283788 |
| SLC2A4 | 1.394821 | 0.214496 |
| C16orf53 | 1.392853 | 0.18717 |
| CLOCK | 1.387177 | 0.1655 |
| RUNX1T1 | 1.383064 | 0.123211 |
| CSK | 1.382241 | 0.006775 |
| CIITA | 1.381362 | 0.116989 |
| MYC | 1.3759 | 0.084916 |
| RELA | 1.374278 | 0.138521 |
| MGMT | 1.371708 | 0.071233 |
| DNMT3L | 1.370523 | 0.417233 |
| TUBA1B | 1.369124 | 0.036232 |
| CCDC101 | 1.359121 | 0.136993 |
| MBD3 | 1.357939 | 0.068524 |
| SETD3 | 1.357248 | 0.2859 |
| CHRNE | 1.356685 | 0.276021 |
| GLI3 | 1.355936 | 0.081031 |
| NQO1 | 1.350978 | 0.255378 |
| AKT1 | 1.350557 | 0.016548 |
| SENP1 | 1.349455 | 0.043645 |
| DSG1 | 1.341291 | 0.456941 |
| SF3B3 | 1.33678 | 0.036031 |
| ARID1A | 1.335636 | 0.100065 |
| RHOB | 1.335009 | 0.169666 |
| SMAD7 | 1.332608 | 0.035266 |
| TGFB1 | 1.328402 | 0.030598 |
| KAT7 | 1.325775 | 0.355004 |
| TUBA8 | 1.321374 | 0.163435 |
| MECOM | 1.319809 | 0.105974 |
| HR | 1.31965 | 0.056254 |
| AES | 1.317987 | 0.11677 |
| PRF1 | 1.308304 | 0.089703 |
| ANKRA2 | 1.305973 | 0.328053 |
| CXXC1 | 1.304489 | 0.16769 |
| KDM1A | 1.301986 | 0.327669 |
| TRAF6 | 1.299649 | 0.168962 |
| SDC1 | 1.294594 | 0.000635 |
| YEATS2 | 1.288982 | 0.188225 |
| SP1 | 1.288461 | 0.087082 |
| CDK2 | 1.288352 | 0.336452 |
| CD7 | 1.284259 | 0.044682 |
| CECR2 | 1.283142 | 0.18675 |
| RUNX1 | 1.282277 | 0.030793 |
| MIER1 | 1.281159 | 0.045202 |
| BRD1 | 1.280489 | 0.153395 |
| SOD2 | 1.276576 | 0.146712 |
| MAGEA1 | 1.27447 | 0.278228 |
| TAL1 | 1.273042 | 0.146306 |
| TUBA4A | 1.272406 | 0.271077 |
| TADA2B | 1.268166 | 0.555467 |
| EP400 | 1.265391 | 0.157126 |
| HPSE | 1.26532 | 0.003101 |
| EDF1 | 1.264755 | 0.102171 |
| PRDX1 | 1.262873 | 0.270256 |
| APC | 1.261094 | 0.027812 |
| DEK | 1.259664 | 0.133109 |
| PKN2 | 1.257164 | 1.030992 |
| RAD9A | 1.252458 | 0.033759 |
| NKX3-1 | 1.249019 | 0.088081 |
| RFXANK | 1.246019 | 0.119515 |
| HIC2 | 1.242777 | 0.067579 |
| PHF21A | 1.241645 | 0.032859 |
| BRD8 | 1.241255 | 0.250153 |
| MTF1 | 1.239474 | 0.133996 |
| VDR | 1.237271 | 0.095719 |
| SETDB1 | 1.236989 | 0.305437 |
| IL5 | 1.235128 | 0.313773 |
| CHD3 | 1.229314 | 0.075268 |
| CSRP2BP | 1.223999 | 0.12218 |
| EZH2 | 1.221456 | 0.081359 |
| ABCC1 | 1.220525 | 0.002991 |
| MYB | 1.213922 | 0.026772 |
| LATS1 | 1.207816 | 0.040248 |
| EHMT1 | 1.206821 | 0.356664 |
| UHRF1 | 1.2045 | 0.199216 |
| BANP | 1.199461 | 0.097482 |
| DIP2B | 1.195987 | 0.195495 |
| BMI1 | 1.194471 | 0.085986 |
| SUPT7L | 1.189915 | 0.191049 |
| ZBTB16 | 1.189615 | 0.159281 |
| F3 | 1.188948 | 0.068143 |
| NCOR1 | 1.18696 | 0.083124 |
| N6AMT2 | 1.186816 | 0.048267 |
| DNMT3B | 1.186363 | 0.176691 |
| HIPK2 | 1.173761 | 0.02301 |
| MYOCD | 1.17372 | 0.018408 |
| ESR1 | 1.171536 | 0.155722 |
| RBBP4 | 1.169651 | 0.077336 |
| BCOR | 1.164514 | 0.125892 |
| PAX6 | 1.159433 | 0.072691 |
| MMP9 | 1.159397 | 0.071555 |
| FES | 1.156963 | 0.126767 |
| ANKRD1 | 1.155667 | 0.028885 |
| SUV39H2 | 1.155644 | 0.079236 |
| LEO1 | 1.152133 | 0.152024 |
| CSNK2A1 | 1.146438 | 0.351157 |
| YWHAB | 1.141826 | 0.125109 |
| E2F6 | 1.141423 | 0.241531 |
| WNK4 | 1.140949 | 0.029076 |
| PHF17 | 1.13958 | 0.094842 |
| CYBB | 1.136424 | 0.119533 |
| EZH1 | 1.135539 | 0.036727 |
| PHF15 | 1.132499 | 0.073218 |
| CDK11A | 1.131643 | 0.057105 |
| JUN | 1.129327 | 0.071908 |
| DPY30 | 1.126064 | 0.067294 |
| TRIM29 | 1.116065 | 0.345501 |
| CXADR | 1.112672 | 0.009816 |
| ARRB1 | 1.112455 | 0.077363 |
| SUN1 | 1.111646 | 0.076219 |
| MAP2K4 | 1.110548 | 0.030478 |
| PYGO2 | 1.110056 | 0.176079 |
| PAXIP1 | 1.109915 | 0.03916 |
| TUBA1A | 1.101101 | 0.275545 |
| KAT6A | 1.100018 | 0.042044 |
| CXCR4 | 1.09997 | 0.070039 |
| TH | 1.097428 | 0.045707 |
| ASH2L | 1.096056 | 0.07783 |
| EFCAB6 | 1.089647 | 0.234229 |
| SP3 | 1.085129 | 0.034565 |
| SAP30 | 1.082599 | 0.120729 |
| PRMT1 | 1.081514 | 0.237139 |
| DDX53 | 1.079228 | 0.001058 |
| DAXX | 1.079173 | 0.154447 |
| TGFBR1 | 1.072997 | 0.021561 |
| VIM | 1.072353 | 0.129505 |
| CDC73 | 1.072147 | 0.002627 |
| CCNB1 | 1.067277 | 0.278213 |
| VEGFA | 1.063288 | 0.1051 |
| CBX4 | 1.060953 | 0.122965 |
| PTPN6 | 1.059981 | 0.118723 |
| CD70 | 1.059567 | 0.241441 |
| ARID4B | 1.059367 | 0.248975 |
| HDAC11 | 1.058263 | 0.346318 |
| ETV6 | 1.055826 | 0.076005 |
| PRKDC | 1.055754 | 0.201604 |
| SUZ12 | 1.055297 | 0.071324 |
| NRIP1 | 1.046621 | 0.119775 |
| CXCL12 | 1.044301 | 0.010749 |
| JARID2 | 1.041966 | 0.485277 |
| NSUN5 | 1.041342 | 0.121705 |
| NFE2L2 | 1.041249 | 0.049485 |
| HMGA2 | 1.038839 | 0.158746 |
| TUBA3C | 1.038519 | 0.095052 |
| AURKB | 1.034848 | 0.06488 |
| TAF10 | 1.033087 | 0.216131 |
| ABCB1 | 1.028707 | 0.254008 |
| RBBP7 | 1.025944 | 0.242586 |
| PRDM14 | 1.023945 | 0.020073 |
| PWWP2B | 1.02175 | 0.245001 |
| MGEA5 | 1.021146 | 0.342213 |
| BRMS1L | 1.020885 | 0.117824 |
| NFKB2 | 1.020123 | 0.088886 |
| TBL1XR1 | 1.019141 | 0.105209 |
| EPAS1 | 1.019089 | 0.11712 |
| HDAC3 | 1.015263 | 0.134457 |
| IL13 | 1.010273 | 0.161229 |
| PEX14 | 1.007146 | 0.079884 |
| SIRT2 | 1.006013 | 0.067993 |
| RELN | 1.005239 | 0.010346 |
| CASP2 | 1.004814 | 0.09785 |
| HIST4H4 | 1.003723 | 0.116332 |
| GADD45A | 1.003043 | 0.049633 |
| SIRT1 | 0.993451 | 0.156621 |
| PAF1 | 0.992577 | 0.252626 |
| KPNA2 | 0.992185 | 0.144404 |
| PRMT5 | 0.99125 | 0.070388 |
| HDAC9 | 0.988737 | 0.29638 |
| MCRS1 | 0.988526 | 0.27915 |
| CDK5R1 | 0.984716 | 0.180926 |
| WHSC1 | 0.984353 | 0.057862 |
| BHLHE41 | 0.980776 | 0.089288 |
| ULBP2 | 0.980401 | 0.080637 |
| SAP18 | 0.977365 | 0.336118 |
| TPPP | 0.977044 | 0.120371 |
| KDM4A | 0.975707 | 0.216738 |
| PRMT7 | 0.974304 | 0.080135 |
| ORC1 | 0.972961 | 0.227276 |
| TUBA1C | 0.958293 | 0.009863 |
| HDAC1 | 0.954666 | 0.037891 |
| SUPT3H | 0.954629 | 0.005615 |
| SIN3B | 0.954596 | 0.034148 |
| AR | 0.954527 | 0.153256 |
| SMYD3 | 0.953981 | 0.062143 |
| HRAS | 0.952721 | 0.03968 |
| DNMT3A | 0.949286 | 0.077614 |
| PHB | 0.944421 | 0.0786 |
| POU2F1 | 0.943371 | 0.205946 |
| TOP2A | 0.941112 | 0.177447 |
| MED24 | 0.939721 | 0.118972 |
| HAT1 | 0.938107 | 0.133348 |
| MBD5 | 0.937029 | 0.053246 |
| PAEP | 0.93251 | 0.087624 |
| TGFBR2 | 0.931022 | 0.164776 |
| HOXA10 | 0.92795 | 0.242772 |
| EPC1 | 0.924411 | 0.272768 |
| PRMT6 | 0.918015 | 0.123808 |
| BPTF | 0.917084 | 0.051215 |
| BRPF1 | 0.916583 | 0.081207 |
| AMD1 | 0.914359 | 0.043902 |
| TADA2A | 0.913515 | 0.001343 |
| N6AMT1 | 0.912432 | 0.153556 |
| H2AFX | 0.909985 | 0.074401 |
| TNFSF10 | 0.90863 | 0.041848 |
| PHF20 | 0.907036 | 0.132451 |
| CARM1 | 0.906589 | 0.164824 |
| C10orf90 | 0.904815 | 0.108391 |
| GCM1 | 0.895003 | 0.093703 |
| GATA3 | 0.889519 | 0.132912 |
| MIF | 0.887288 | 0.202184 |
| MLL4 | 0.884682 | 0.18084 |
| ATG7 | 0.884404 | 0.257644 |
| RUNX2 | 0.878571 | 0.179591 |
| CDKN2A | 0.876777 | 0.04081 |
| TRDMT1 | 0.874763 | 0.316913 |
| PRKCB | 0.865363 | 0.027988 |
| ELP3 | 0.852046 | 0.002506 |
| SNAI1 | 0.848042 | 0.47434 |
| NFYA | 0.846109 | 0.069178 |
| SKI | 0.840185 | 0.317046 |
| STAT3 | 0.837697 | 0.000411 |
| TAF1L | 0.828767 | 0.092826 |
| EGR1 | 0.825164 | 0.319625 |
| PCNA | 0.822489 | 0.032645 |
| HDAC7 | 0.81853 | 0.062524 |
| ATM | 0.818324 | 0.130596 |
| PWWP2A | 0.817376 | 0.200661 |
| GPS2 | 0.814992 | 0.130853 |
| SIRT6 | 0.81375 | 0.163215 |
| MLL5 | 0.812817 | 0.00239 |
| HDAC6 | 0.810317 | 0.172632 |
| OGT | 0.807573 | 0.272133 |
| HDAC10 | 0.801948 | 0.060865 |
| HDLBP | 0.79396 | 0.150848 |
| HINFP | 0.792256 | 0.097991 |
| MEN1 | 0.791006 | 0.103989 |
| PDK4 | 0.789981 | 0.081937 |
| CBFA2T2 | 0.787079 | 0.059737 |
| TBL1X | 0.780768 | 0.370097 |
| MTA1 | 0.775538 | 0.133515 |
| LOXL1 | 0.773899 | 0.034506 |
| DUSP4 | 0.772888 | 0.046944 |
| SPOP | 0.769197 | 0.026385 |
| CAMTA2 | 0.768023 | 0.053034 |
| CTNNB1 | 0.765634 | 0.082771 |
| HEY2 | 0.752609 | 0.039083 |
| SUDS3 | 0.752453 | 0.075844 |
| CTAG1B | 0.747344 | 0.023073 |
| PPARG | 0.736318 | 0.024536 |
| ING4 | 0.73434 | 0.005399 |
| KIAA1310 | 0.728351 | 0.138733 |
| SETD7 | 0.724616 | 0.168574 |
| NFYC | 0.722623 | 0.236815 |
| SETD2 | 0.719231 | 0.02749 |
| NFATC1 | 0.714875 | 0.19817 |
| KAT8 | 0.713947 | 0.023441 |
| HDAC4 | 0.704703 | 0.141005 |
| BAZ1A | 0.704298 | 0.066868 |
| HBB | 0.687953 | 0.233731 |
| ACTB | 0.67941 | 0.190259 |
| TRIM68 | 0.659491 | 0.022622 |
| CHEK1 | 0.647456 | 0.039642 |
| HMG20B | 0.6231 | 0.130657 |
| RBBP5 | 0.609692 | 0.01255 |
| TAF5 | 0.602427 | 0.048666 |
| ZBTB7A | 0.598118 | 0.001466 |
| YY1 | 0.585861 | 0.275667 |
| KIAA1267 | 0.584697 | 0.076867 |
| ATF2 | 0.573167 | 0.005057 |
| ING3 | 0.567088 | 0.011395 |
| MAPT | 0.559047 | 0.103793 |
| AURKA | 0.558069 | 0.097153 |
| SETD8 | 0.554762 | 0.035052 |
| ARID4A | 0.55208 | 0.040552 |
| STAT1 | 0.54645 | 0.042275 |
| C12orf41 | 0.538001 | 0.078301 |
| TAF1 | 0.523895 | 0.025411 |
| HES1 | 0.506581 | 0.206968 |
| CTBP1 | 0.473647 | 0.187538 |
| WDR5 | 0.462155 | 0.016306 |
| SAP30L | 0.457775 | 0.119981 |
| HCFC1 | 0.410236 | 0.062887 |
| SAP130 | 0.331476 | 0.085623 |
| SIN3A | 0.309358 | 0.015005 |
| CTCF | 0.164847 | 0.071473 |
| RUNX3 | No data | No data |

Gene: Target of an siRNA,
Fold change: Fold change in FXN mRNA expression compared to control,
STDEV: standard deviation

### Example 3

### Upregulation of FXN Expression in Cells Treated with a Histone Lysine Methyltransferase Inhibitor

### Epigenetic Inhibitors

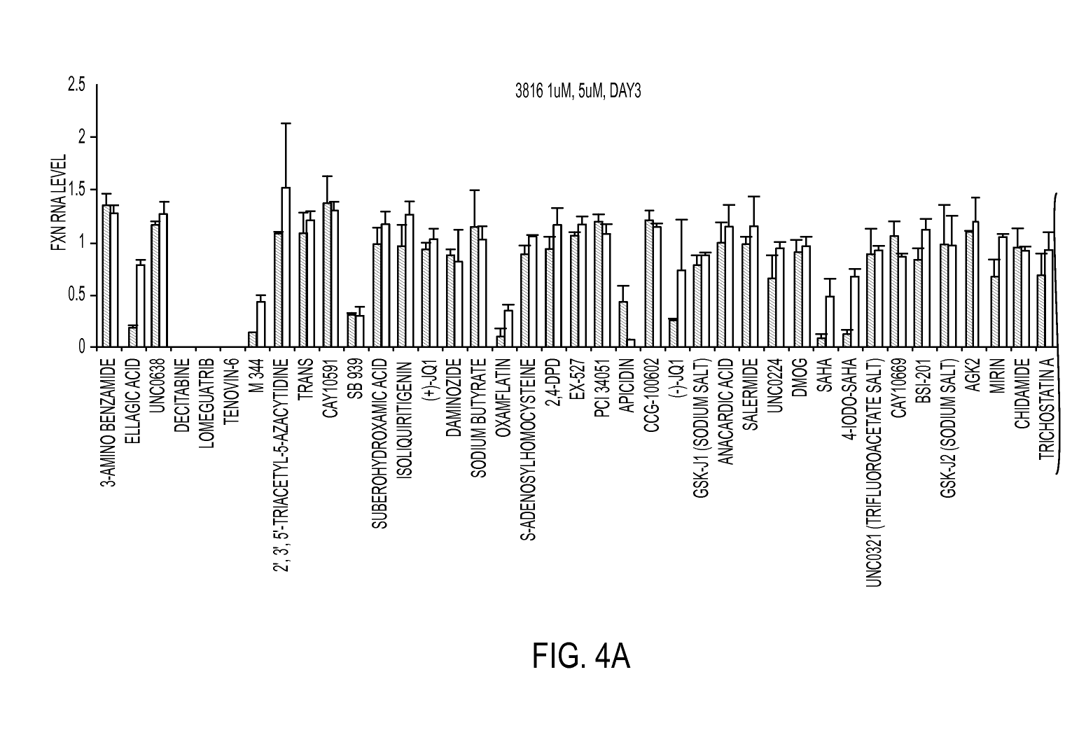

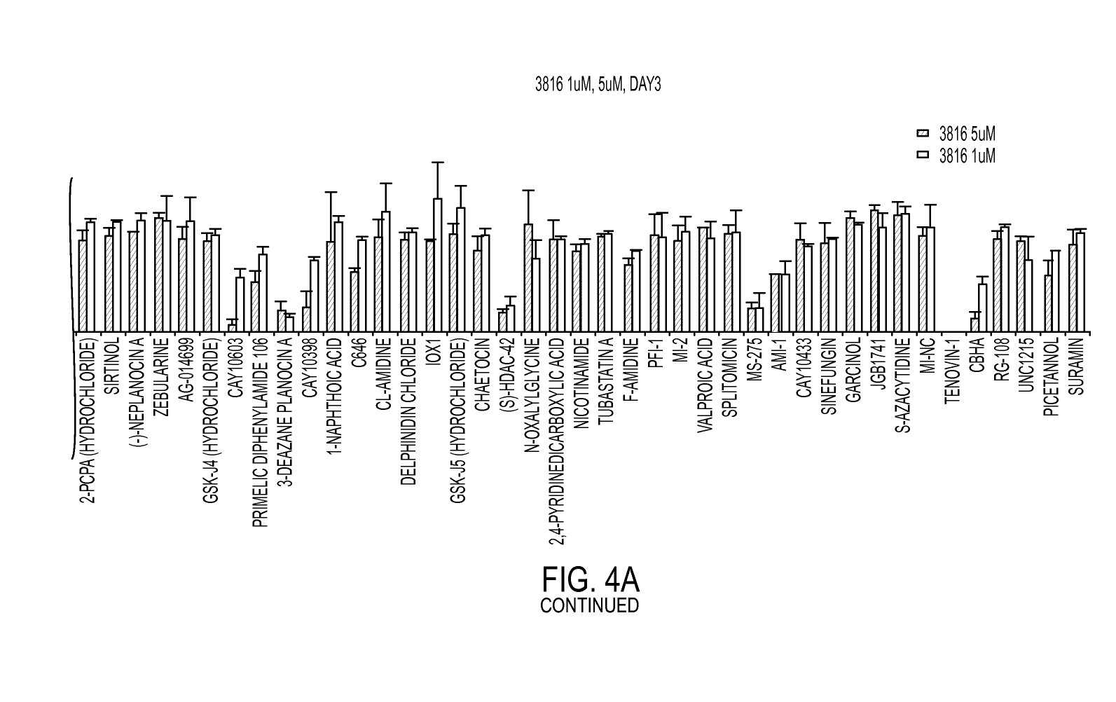

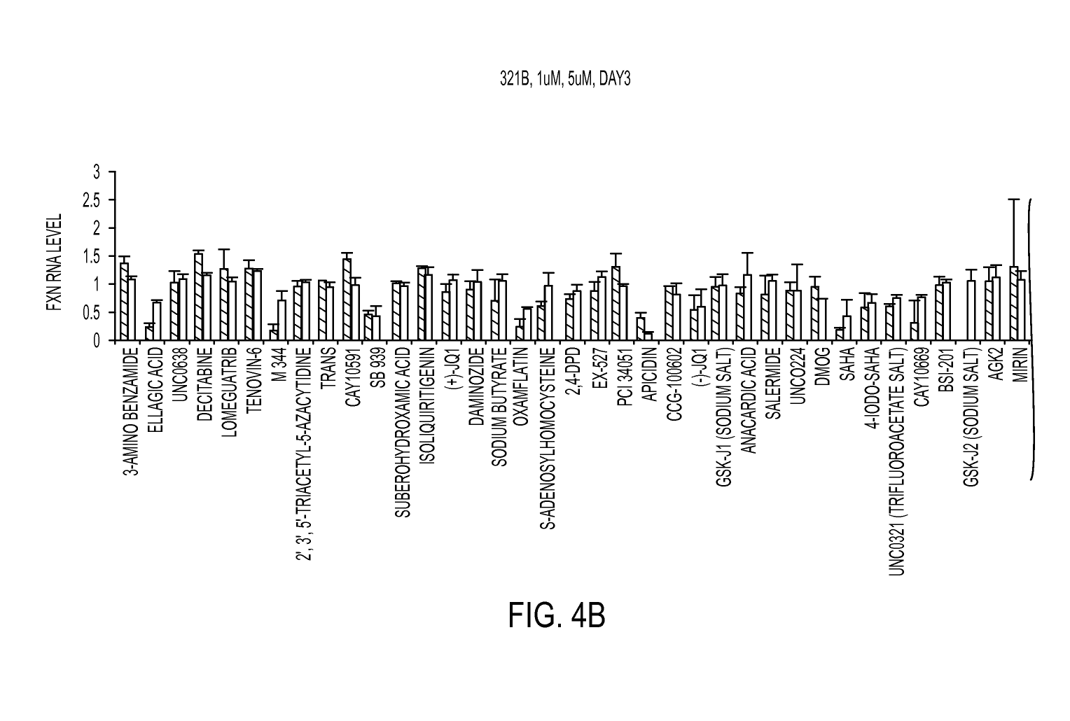

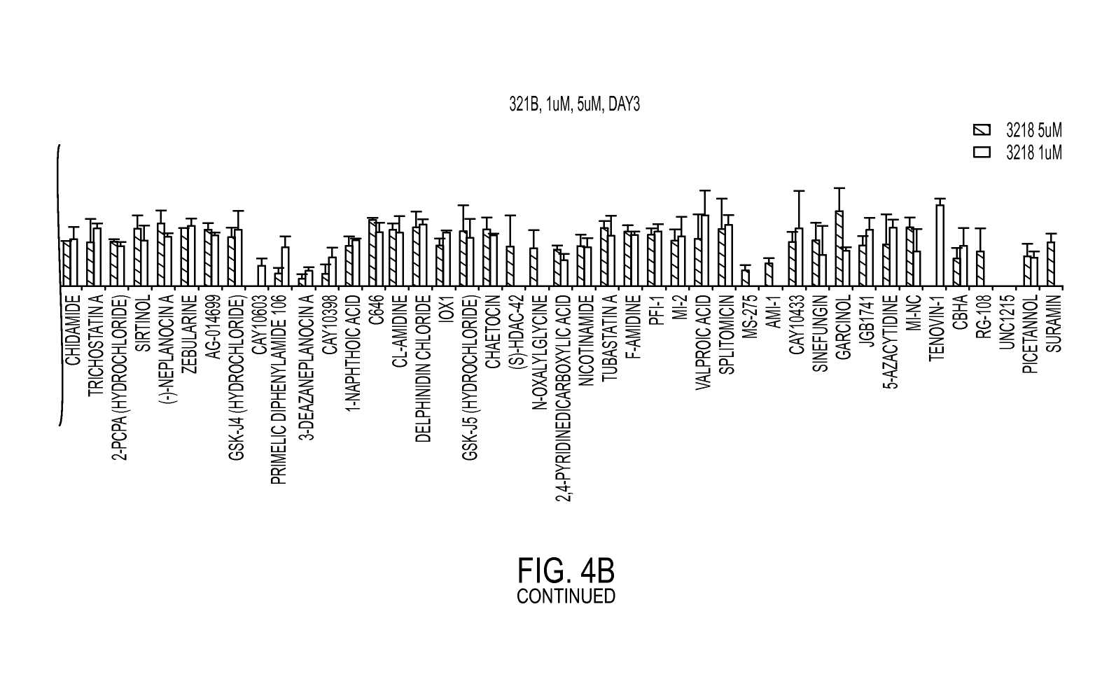

A screen of a library of eighty epigenetic inhibitors (Cayman Chemical) was performed in GM03816 FRDA fibroblasts to identify epigenetic regulators that upregulate FXN expression. The results of the screen are provided in FIG. 4 and Tables 10 and 11. The data shows FXN mRNA fold changes in response to 1 μM and 5 μM inhibitor treatment following 3 days of treatment.

FXN RNA Measurements in Cells Treated with Histone Lysine Methyltransferase Inhibitors

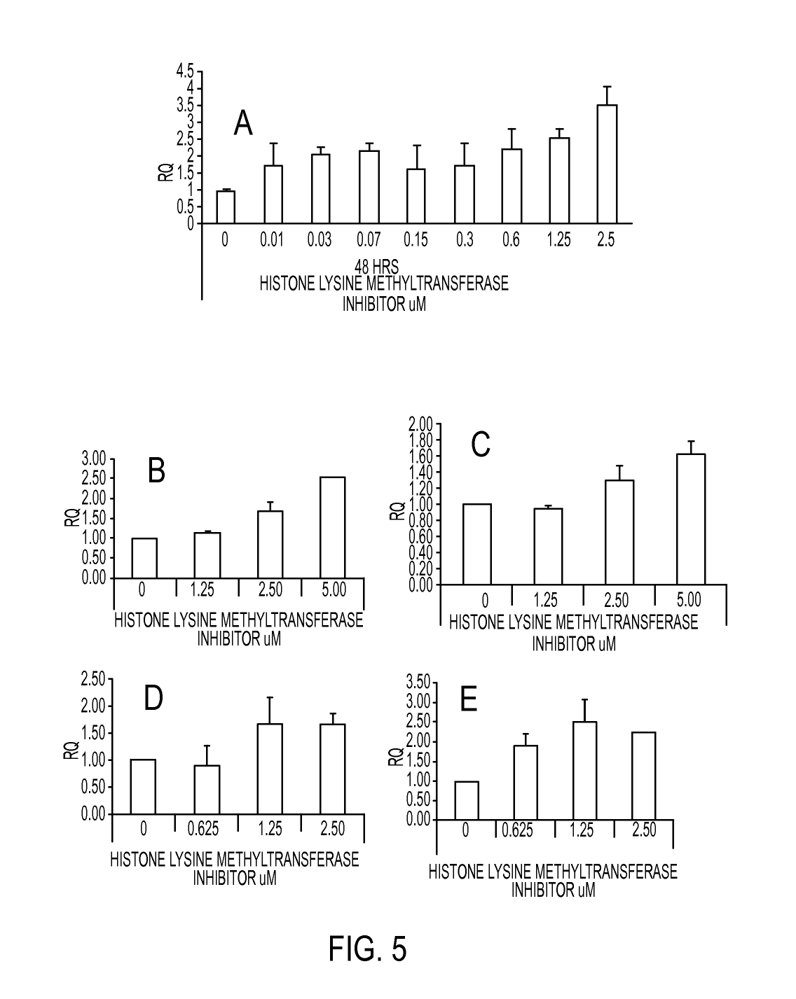

GM03816 and GM04078 cells were plated at 4000 cells/well. Sarsero mouse model derived fibroblasts were plated at 6000/well. Sarsero mouse model (B6.Cg-Tg(FXN)1Sars Fxn^{tm1Mkn}/J; see catalog from The Jackson Laboratory at jaxmice.jax.org/strain/008586.html) was generated by inserting a human BAC containing FXN genomic region with repeat expansion into mouse genome. The resulting Sarsero mouse model and cell lines derived from it expressed mouse FXN and human FXN mRNAs. The histone lysine methyltranferase inhibitor, 2-(Hexahydro-4-methyl-1H-1,4-diazepin-1-yl)-6,7-dimethoxy-N-(1-(phenylmethyl)-4-piperidinyl)-4-quinazolinamine, was dissolved in DMSO and cells were treated at various concentrations and times shown in FIG. 5A-5E. Cells-to-Ct (Life Technologies) procedure was used to analyze RNA levels of FXN following manufacturer's protocol. The Taqman probes used were from Life Technologies are: FXN Hs00175940_m1, Actin Hs01060665_g1, Gapdh Hs02758991_g1, Gusb Hs00939627_m1, PPIB Hs00168719_m1, HPRT1 Hs01003267_m1.

FXN Protein Measurements in Cells Treated with Histone Lysine Methyltransferase Inhibitors

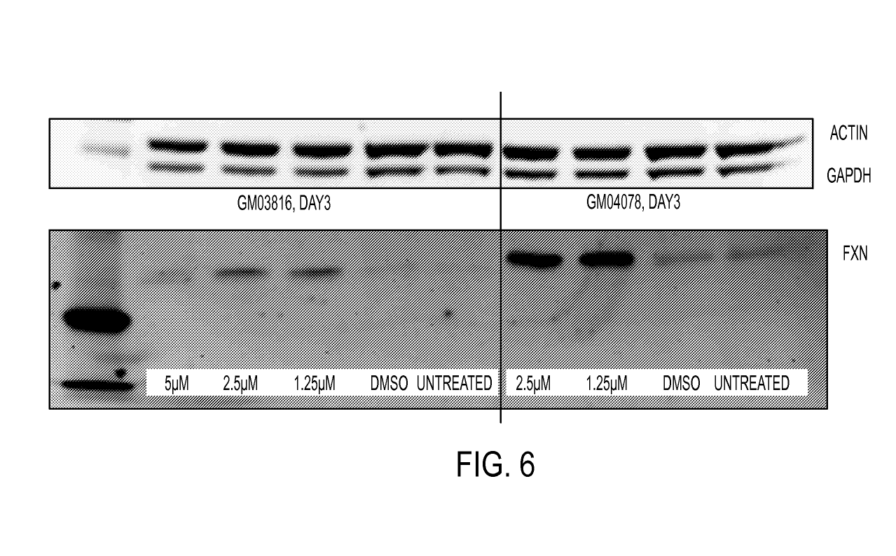

Human FRDA diseased cell lines GM03816 and GM04078 were plated at 150000 cells/well. The cells were treated at various concentrations and times with a histone lysine methyltransferase inhibitor dissolved in DMSO. The antibody used for detection of FXM protein was Abcam human FXN antibody (ab48281). FIG. 6 shows that 3 days of HLMi treatment of human FRDA diseased cell lines GM03816 and GM04078 result in FXN human protein upregulation.

TABLE 10
Data from screen using GM03816 FRDA diseased fibroblasts

| Agent | 3816 5 uM | stdev | 3816 1 uM | stdev |
|---|---|---|---|---|
| 3-amino Benzamide | 1.357058458 | 0.105939139 | 1.289386933 | 0.063408013 |
| Ellagic Acid | 0.189498667 | 0.018586462 | 0.788222048 | 0.048405731 |
| UNC0638 | 1.169575316 | 0.027763557 | 1.270873138 | 0.115930289 |
| Decitabine | UD | UD | UD | UD |
| Lomeguatrib | UD | UD | UD | UD |
| Tenovin-6 | UD | UD | UD | UD |
| M 344 | 0.140039867 | 0.002638068 | 0.439008339 | 0.061045102 |
| 2′,3′,5′-triacetyl-5- | 1.090385376 | 0.013661266 | 1.529058078 | 0.605165289 |
| Azacytidine | | | | |
| trans | 1.091704774 | 0.19667012 | 1.213873223 | 0.085824951 |
| CAY10591 | 1.377307853 | 0.256002786 | 1.311005747 | 0.080510552 |
| SB 939 | 0.314738414 | 0.01103722 | 0.297774901 | 0.093518952 |
| Suberohydroxamic Acid | 0.982446519 | 0.160322671 | 1.175958515 | 0.124049261 |
| Isoliquiritigenin | 0.962642999 | 0.203440779 | 1.274104348 | 0.119334079 |
| (+)-JQ1 | 0.93841674 | 0.060871039 | 1.036695152 | 0.097224531 |
| Daminozide | 0.881052964 | 0.057150098 | 0.82384919 | 0.298986999 |
| Sodium Butyrate | 1.156398821 | 0.350188559 | 1.034100346 | 0.116138665 |
| Oxamflatin | 0.107990912 | 0.067581086 | 0.350603686 | 0.053979587 |
| S-Adenosylhomocysteine | 0.886498805 | 0.086084969 | 1.066820322 | 0.003856229 |
| 2,4-DPD | 0.945561423 | 0.110712384 | 1.179139924 | 0.150788127 |
| EX-527 | 1.071782266 | 0.027083761 | 1.176537129 | 0.068798948 |
| PCI 34051 | 1.209377942 | 0.063136653 | 1.083645505 | 0.09157642 |
| Apicidin | 0.434910853 | 0.152083815 | 0.079096291 | 0.002766458 |
| CCG-100602 | 1.212762649 | 0.090600593 | 1.14724342 | 0.036191887 |
| (−)-JQ1 | 0.25511335 | 0.016048968 | 0.742213224 | 0.480450992 |
| GSK-J1 (sodium salt) | 0.789151407 | 0.083942759 | 0.886633027 | 0.014502947 |
| Anacardic Acid | 1.009764707 | 0.181421749 | 1.162010476 | 0.195769117 |
| Salermide | 0.996206778 | 0.058284207 | 1.164658132 | 0.269625279 |
| UNC0224 | 0.655624418 | 0.225943318 | 0.957749577 | 0.04803681 |
| DMOG | 0.913144669 | 0.108750574 | 0.968521291 | 0.081256263 |
| SAHA | 0.09160427 | 0.041037035 | 0.48573854 | 0.166066317 |
| 4-iodo-SAHA | 0.130243602 | 0.039806546 | 0.687836429 | 0.060814441 |
| UNC0321 (trifluoroacetate | 0.892927144 | 0.243500789 | 0.932277376 | 0.03534697 |
| salt) | | | | |
| CAY10669 | 1.073941455 | 0.127900599 | 0.87089951 | 0.023634093 |
| BSI-201 | 0.843668592 | 0.098833864 | 1.125801896 | 0.096238551 |
| GSK-J2 (sodium salt) | 0.993767738 | 0.366135225 | 0.971069284 | 0.28533974 |
| AGK2 | 1.101357256 | 0.001855317 | 1.206247755 | 0.225478637 |
| Mirin | 0.679489952 | 0.157893195 | 1.057750106 | 0.02404031 |
| Chidamide | 0.967882215 | 0.165489129 | 0.924187158 | 0.032437385 |
| Trichostatin A | 0.692090238 | 0.209259791 | 0.93376589 | 0.17184603 |
| 2-PCPA (hydrochloride) | 0.878612395 | 0.086658859 | 1.057774688 | 0.02611407 |
| Sirtinol | 0.919798649 | 0.078151446 | 1.055841853 | 0.015847805 |
| (−)-Neplanocin A | 0.94296969 | 0.002108911 | 1.067286134 | 0.071807707 |
| Zebularine | 1.092659413 | 0.043133835 | 1.067746106 | 0.235460396 |
| AG-014699 | 0.892105436 | 0.114867995 | 1.06102006 | 0.219749097 |
| GSK-J4 (hydrochloride) | 0.876192868 | 0.06583111 | 0.927049179 | 0.057838291 |
| CAY10603 | 0.070044821 | 0.054894784 | 0.520549919 | 0.085497828 |
| Pimelic Diphenylamide 106 | 0.485794097 | 0.090529435 | 0.745731428 | 0.072483905 |
| 3-Deazaneplanocin A | 0.210248073 | 0.083585131 | 0.151359548 | 0.027764356 |
| CAY10398 | 0.233878822 | 0.156585213 | 0.692101588 | 0.031409382 |
| 1-Naphthoic Acid | 0.868571662 | 0.46279875 | 1.054960967 | 0.052008788 |
| C646 | 0.579816754 | 0.035944263 | 0.871311642 | 0.044661075 |
| Cl-Amidine | 0.902809074 | 0.175193443 | 1.152685863 | 0.264103724 |
| Delphinidin chloride | 0.884641057 | 0.063871379 | 0.953502066 | 0.035334428 |
| IOX1 | 0.872237598 | 0.00494326 | 1.271688367 | 0.350500763 |
| GSK-J5 (hydrochloride) | 0.945088335 | 0.089468976 | 1.183072281 | 0.212452207 |
| Chaetocin | 0.777759373 | 0.132934472 | 0.930191108 | 0.065426579 |
| (S)-HDAC-42 | 0.189368291 | 0.037760292 | 0.241864641 | 0.095041055 |
| N-Oxalylglycine | 1.034288774 | 0.313210364 | 0.707956911 | 0.167271222 |
| 2,4-Pyridinedicarboxylic | 0.887726645 | 0.17653368 | 0.884632145 | 0.027040586 |
| Acid | | | | |
| Nicotinamide | 0.775449369 | 0.068537188 | 0.852389108 | 0.039413998 |
| Tubastatin A | 0.919227107 | 0.022271163 | 0.927598152 | 0.042551002 |
| F-Amidine | 0.648423217 | 0.056320466 | 0.781768651 | 0.001388982 |
| PFI-1 | 0.922957467 | 0.205213618 | 0.917510237 | 0.217110895 |
| MI-2 | 0.873563465 | 0.146408285 | 0.966216849 | 0.135292559 |
| Valproic acid | 1.00366769 | 0.000706899 | 0.900512098 | 0.153127208 |
| Splitomicin | 0.938571429 | 0.081980311 | 0.957073894 | 0.205902804 |
| MS-275 | 0.233294275 | 0.039550745 | 0.226635876 | 0.14653073 |
| AMI-1 | 0.565963811 | 0.000433569 | 0.55791848 | 0.119762235 |
| CAY10433 | 0.882676256 | 0.151293133 | 0.830938143 | 0.000967259 |
| Sinefungin | 0.861913953 | 0.176780362 | 0.894909785 | 0.001918967 |
| Garcinol | 1.094467111 | 0.056066205 | 1.008756637 | 0.0273752 |
| JGB1741 | 1.169911294 | 0.048402996 | 1.006483881 | 0.124181838 |
| 5-azacytidine | 1.110725939 | 0.126874492 | 1.13359584 | 0.062404931 |
| MI-nc | 0.924552089 | 0.077595364 | 1.002692191 | 0.207187812 |
| tenovin-1 | UD | UD | UD | UD |
| CBHA | 0.132678455 | 0.062288615 | 0.467814763 | 0.061188269 |
| RG-108 | 0.89656673 | 0.069990726 | 1.001076646 | 0.02581795 |
| UNC1215 | 0.874283351 | 0.043449042 | 0.691060773 | 0.220171187 |
| Picetannol | 0.543519173 | 0.137963482 | 0.786661105 | 0.001686848 |
| Suramin | 0.835317044 | 0.142772218 | 0.954129468 | 0.033488309 |

UD = undetermined

TABLE 11
Data from screen using GM0321 normal fibroblasts

| Agent | 321B 5 uM | stdev | 321B 1 uM | stdev |
|---|---|---|---|---|
| 3-amino Benzamide | 1.365421477 | 0.123805307 | 1.088829696 | 0.03901872 |
| Ellagic Acid | 0.247513256 | 0.052167582 | 0.67475149 | 0.027815198 |
| UNC0638 | 1.023533057 | 0.201972766 | 1.094572256 | 0.071767316 |
| Decitabine | 1.532477156 | 0.056508338 | 1.15261439 | 0.046802866 |
| Lomeguatrib | 1.280122846 | 0.330210306 | 1.051726264 | 0.066519738 |
| Tenovin-6 | 1.282124175 | 0.137513526 | 1.230434979 | 0.038511916 |
| M 344 | 0.181643964 | 0.099129192 | 0.709765777 | 0.153633556 |
| 2′,3′,5′-triacetyl-5- | 0.958029413 | 0.091074106 | 1.035528777 | 0.04052709 |
| Azacytidine | | | | |
| trans | 1.058033983 | 0.005833919 | 0.951802276 | 0.077880064 |
| CAY10591 | 1.436624341 | 0.114826946 | 0.982784766 | 0.122574646 |
| SB 939 | 0.464956661 | 0.058808663 | 0.434519993 | 0.166758827 |
| Suberohydroxamic Acid | 1.023486458 | 0.01843429 | 0.959104061 | 0.060661555 |
| Isoliquiritigenin | 1.287363832 | 0.014354371 | 1.160719136 | 0.132340316 |
| (+)-JQ1 | 0.859719214 | 0.135850013 | 1.069873736 | 0.091198943 |
| Daminozide | 0.897590624 | 0.154418615 | 1.039533894 | 0.201071974 |
| Sodium Butyrate | 0.706874263 | 0.373013261 | 1.063888474 | 0.09744099 |
| Oxamflatin | 0.239116409 | 0.129244928 | 0.561244925 | 0.018391471 |
| S-Adenosylhomocysteine | 0.619357747 | 0.068842916 | 0.975272402 | 0.213904215 |
| 2,4-DPD | 0.737211525 | 0.080146885 | 0.877012936 | 0.111514801 |
| EX-527 | 0.8741177 | 0.162184635 | 1.135923277 | 0.085585858 |
| PCI 34051 | 1.305690309 | 0.226517996 | 0.974305357 | 0.018560677 |
| Apicidin | 0.397352432 | 0.090875561 | 0.119436545 | 0.015999276 |
| CCG-100602 | 0.95920826 | 0.010225261 | 0.832495695 | 0.178102726 |
| (−)-JQ1 | 0.547035828 | 0.24438329 | 0.58748671 | 0.312211583 |
| GSK-J1 (sodium salt) | 0.942264129 | 0.173466759 | 0.977918426 | 0.2046503 |
| Anacardic Acid | 0.835640147 | 0.098986232 | 1.168894153 | 0.383135062 |
| Salermide | 0.81494605 | 0.333045164 | 1.063741493 | 0.094176986 |
| UNC0224 | 0.874937277 | 0.151366306 | 0.876080902 | 0.468527904 |
| DMOG | 0.943307215 | 0.190899522 | 0.731723103 | 0.009012364 |
| SAHA | 0.192332256 | 0.016922721 | 0.425634261 | 0.280691833 |
| 4-iodo-SAHA | 0.590087532 | 0.242476585 | 0.667981929 | 0.152571564 |
| UNC0321 (trifluoroacetate | 0.605016162 | 0.034453671 | 0.751678309 | 0.046343556 |
| salt) | | | | |
| CAY10669 | 0.30939022 | 0.394654889 | 0.779613873 | 0.005299845 |
| BSI-201 | 0.985335599 | 0.139941948 | 1.027793404 | 0.046394853 |
| GSK-J2 (sodium salt) | UD | UD | 1.062992432 | 0.187553234 |
| AGK2 | 1.052526568 | 0.242975783 | 1.115129842 | 0.217839055 |
| Mirin | 1.305145804 | 1.197784414 | 1.078594045 | 0.148150954 |
| Chidamide | 0.79851937 | 0.0065555 | 0.835394192 | 0.203443672 |
| Trichostatin A | 0.772139968 | 0.41928721 | 1.022984586 | 0.084573946 |
| 2-PCPA (hydrochloride) | 0.788826909 | 0.039516071 | 0.714929222 | 0.053957012 |
| Sirtinol | 1.024038665 | 0.229799104 | 0.809656129 | 0.256257926 |
| (−)-Neplanocin A | 1.104954517 | 0.228649677 | 0.873369703 | 0.062499549 |
| Zebularine | 1.030528935 | 0.005429716 | 1.070144275 | 0.125660328 |
| AG-014699 | 1.000307564 | 0.112404334 | 0.89512822 | 0.04347404 |
| GSK-J4 (hydrochloride) | 0.874380992 | 0.161812285 | 1.001650686 | 0.323202006 |
| CAY10603 | UD | UD | 0.359151916 | 0.124031472 |
| Pimelic Diphenylamide 106 | 0.209357829 | 0.103834077 | 0.694010453 | 0.178324186 |
| 3-Deazaneplanocin A | 0.126529782 | 0.075077401 | 0.264148931 | 0.061938623 |
| CAY10398 | 0.210602925 | 0.157813128 | 0.504871673 | 0.169165631 |
| 1-Naphthoic Acid | 0.719662274 | 0.150818584 | 0.819305969 | 0.017213854 |
| C646 | 1.194906621 | 0.00951685 | 0.9473841 | 0.173551083 |
| Cl-Amidine | 0.993094863 | 0.112319322 | 0.943203062 | 0.291605431 |
| Delphinidin chloride | 1.036030794 | 0.2792543 | 1.087397481 | 0.100124886 |
| IOX1 | 0.718470223 | 0.119958931 | 0.947879144 | 0.033846977 |
| GSK-J5 (hydrochloride) | 0.974105226 | 0.452288393 | 0.859683667 | 0.325237732 |
| Chaetocin | 0.995203166 | 0.21904671 | 0.908355762 | 0.005729835 |
| (S)-HDAC-42 | 0.69412966 | 0.551769336 | UD | UD |
| N-Oxalylglycine | 0.680352211 | 0.307987658 | UD | UD |
| 2,4-Pyridinedicarboxylic | 0.645136143 | 0.061643985 | 0.458469394 | 0.112579758 |
| Acid | | | | |
| Nicotinamide | 0.701881607 | 0.206529425 | 0.686626406 | 0.171214858 |
| Tubastatin A | 1.037805434 | 0.111309254 | 0.899268368 | 0.344604097 |
| F-Amidine | 0.969736884 | 0.117230202 | 0.915167018 | 0.054748715 |
| PFI-1 | 0.907082488 | 0.100382273 | 0.957719401 | 0.126896814 |
| MI-2 | 0.796570274 | 0.20585434 | 0.880135212 | 0.347344616 |
| Valproic acid | 0.846928872 | 0.426766495 | 1.263618913 | 0.42865371 |
| Splitomicin | 1.005944436 | 0.545406716 | 1.082820732 | 0.174970499 |
| MS-275 | 0.271301857 | 0.096951699 | UD | UD |
| AMI-1 | 0.407259491 | 0.094404401 | UD | UD |
| CAY10433 | 0.778212312 | 0.187992917 | 1.03043719 | 0.643456098 |
| Sinefungin | 0.821339027 | 0.302536905 | 0.550738646 | 0.507012577 |
| Garcinol | 1.327373705 | 0.403942211 | 0.629526279 | 0.057578097 |
| JGB1741 | 0.722420283 | 0.164874607 | 0.996271558 | 0.197368868 |
| 5-azacytidine | 0.753502134 | 0.516661665 | 1.045672178 | 0.124313534 |
| MI-nc | 1.043599944 | 0.17903364 | 0.613264168 | 0.386029314 |
| tenovin-1 | UD | UD | 1.444268369 | 0.10811216 |
| CBHA | 0.48745022 | 0.185880937 | 0.720833131 | 0.299071952 |
| RG-108 | 0.622247164 | 0.398108985 | UD | UD |
| UNC1215 | UD | UD | UD | UD |
| Picetannol | 0.537994883 | 0.205766438 | 0.500760875 | 0.09776034 |
| Suramin | 0.772191365 | 0.146805196 | UD | UD |

UD = undetermined

### Example 4

### Data for Gapmers to JunD, YEATS4, HIC1, ACTL6A, EID1, IDH1, TNFSF9, JAK2, KAT2A and PRKCD

Gapmers for human JunD, YEATS4, HIC1, ACTL6A, EID1, IDH1, TNFSF9, JAK2, KAT2A and PRKCD were designed against the genes identified within the epigenetic siRNA screen, whose knockdown was hypothesized to lead to FXN mRNA upregulation. The oligo sequences are shown in Table 12.

TABLE 12
Gapmers targeting various genes identified with the epigenetic siRNA screen.

| SEQ | Oligo | Base | Gene | | |
|---|---|---|---|---|---|
| ID NO | Name | Sequence | Name | Organism | Formatted Sequence |
| 92 | JUND-01 | CCGTAGAAGGG | JUND | human | InaCs;InaCs;InaGs;dTs;dAs;dGs;dAs;d |
| | m08 | TGTT | | | As;dGs;dGs;dGs;dTs;InaGs;InaTs;InaT- |
| | | | | | Sup |
| 93 | JUND-02 | TTCATCATGCTG | JUND | human | InaTs;InaTs;InaCs;dAs;dTs;dCs;dAs;dT |
| | m08 | CCG | | | s;dGs;dCs;dTs;dGs;InaCs;InaCs;InaG- |
| | | | | | Sup |
| 94 | JUND-03 | CTGTGAGCTCG | JUND | human | InaCs;InaTs;InaGs;dTs;dGs;dAs;dGs;d |
| | m08 | TCGG | | | Cs;dTs;dCs;dGs;dTs;InaCs;InaGs;InaG- |
| | | | | | Sup |
| 95 | JUND-04 | GGAACTGTGAG | JUND | human | InaGs;InaGs;InaAs;dAs;dCs;dTs;dGs;d |
| | m08 | CTCG | | | Ts;dGs;dAs;dGs;dCs;InaTs;InaCs;InaG- |
| | | | | | Sup |
| 96 | JUND-05 | GCTCGTCCTTGA | JUND | human | InaGs;InaCs;InaTs;dCs;dGs;dTs;dCs;d |
| | m08 | GCG | | | Cs;dTs;dTs;dGs;dAs;InaGs;InaCs;InaG- |
| | | | | | Sup |
| 97 | JUND-06 | TGGCTCGTCCTT | JUND | human | InaTs;InaGs;InaGs;dCs;dTs;dCs;dGs;d |
| | m08 | GAG | | | Ts;dCs;dCs;dTs;dTs;InaGs;InaAs;InaG- |
| | | | | | Sup |
| 98 | JUND-07 | CCCGTTGGACT | JUND | human | InaCs;InaCs;InaCs;dGs;dTs;dTs;dGs;d |
| | m08 | GGAT | | | Gs;dAs;dCs;dTs;dGs;InaGs;InaAs;InaT- |
| | | | | | Sup |
| 99 | JUND-08 | CGCTCCGCCTTG | JUND | human | InaCs;InaGs;InaCs;dTs;dCs;dCs;dGs;d |
| | m08 | ATG | | | Cs;dCs;dTs;dTs;dGs;InaAs;InaTs;InaG- |
| | | | | | Sup |
| 100 | JUND-09 | CACCTGCTCGC | JUND | human | InaCs;InaAs;InaCs;dCs;dTs;dGs;dCs;d |
| | m08 | GCAG | | | Ts;dCs;dGs;dCs;dGs;InaCs;InaAs;InaG- |
| | | | | | Sup |
| 101 | HIC1-01 | GGCCGGTGTAG | HIC1 | human | InaGs;InaGs;InaCs;dCs;dGs;dGs;dTs;d |
| | m08 | ATGA | | | Gs;dTs;dAs;dGs;dAs;InaTs;InaGs;InaA- |
| | | | | | Sup |
| 102 | HIC1-02 | TGACCGCGGCC | HIC1 | human | InaTs;InaGs;InaAs;dCs;dCs;dGs;dCs;d |
| | m08 | TCTG | | | Gs;dGs;dCs;dCs;dTs;InaCs;InaTs;InaG- |
| | | | | | Sup |
| 103 | HIC1-03 | TTGACCGCGGC | HIC1 | human | InaTs;InaTs;InaGs;dAs;dCs;dCs;dGs;d |
| | m08 | CTCT | | | Cs;dGs;dGs;dCs;dCs;InaTs;InaCs;InaT- |
| | | | | | Sup |
| 104 | HIC1-04 | TACCGGTCTCCT | HIC1 | human | InaTs;InaAs;InaCs;dCs;dGs;dGs;dTs;d |
| | m08 | CGC | | | Cs;dTs;dCs;dCs;dTs;InaCs;InaGs;InaC- |
| | | | | | Sup |
| 105 | HIC1-05 | ACGTACAGGTT | HIC1 | human | InaAs;InaCs;InaGs;dTs;dAs;dCs;dAs;d |
| | m08 | GTCA | | | Gs;dGs;dTs;dTs;dGs;InaTs;InaCs;InaA- |
| | | | | | Sup |
| 106 | HIC1-06 | ACACGTACAGG | HIC1 | human | InaAs;InaCs;InaAs;dCs;dGs;dTs;dAs;d |
| | m08 | TTGT | | | Cs;dAs;dGs;dGs;dTs;InaTs;InaGs;InaT- |
| | | | | | Sup |
| 107 | HIC1-07 | TCTTGTCGCACG | HIC1 | human | InaTs;InaCs;InaTs;dTs;dGs;dTs;dCs;d |
| | m08 | ACG | | | Gs;dCs;dAs;dCs;dGs;InaAs;InaCs;InaG- |
| | | | | | Sup |
| 108 | HIC1-08 | AGCTCTTGTCGC | HIC1 | human | InaAs;InaGs;InaCs;dTs;dCs;dTs;dTs;d |
| | m08 | ACG | | | Gs;dTs;dCs;dGs;dCs;InaAs;InaCs;InaG- |
| | | | | | Sup |
| 109 | HIC1-09 | CCGCACGCGTC | HIC1 | human | InaCs;InaCs;InaGs;dCs;dAs;dCs;dGs;d |
| | m08 | GCAC | | | Cs;dGs;dTs;dCs;dGs;InaCs;InaAs;InaC- |
| | | | | | Sup |
| 110 | HIC1-10 | TGTGCGAACTT | HIC1 | human | InaTs;InaGs;InaTs;dGs;dCs;dGs;dAs;d |
| | m08 | GCCG | | | As;dCs;dTs;dTs;dGs;InaCs;InaCs;InaG- |
| | | | | | Sup |
| 111 | HIC1-11 | GCTGTGCGAAC | HIC1 | human | InaGs;InaCs;InaTs;dGs;dTs;dGs;dCs;d |
| | m08 | TTGC | | | Gs;dAs;dAs;dCs;dTs;InaTs;InaGs;InaC- |
| | | | | | Sup |
| 112 | HIC1-12 | TCGAGCTTGCC | HIC1 | human | InaTs;InaCs;InaGs;dAs;dGs;dCs;dTs;d |
| | m08 | CTTG | | | Ts;dGs;dCs;dCs;dCs;InaTs;InaTs;InaG- |
| | | | | | Sup |
| 113 | HIC1-13 | AGAAACGGTCG | HIC1 | human | InaAs;InaGs;InaAs;dAs;dAs;dCs;dGs;d |
| | m08 | ATGG | | | Gs;dTs;dCs;dGs;dAs;InaTs;InaGs;InaG- |
| | | | | | Sup |
| 114 | YEATS4- | TCGGCCATTCTC | YEATS4 | human | InaTs;InaCs;InaGs;dGs;dCs;dCs;dAs;d |
| | 01 m08 | TTG | | | Ts;dTs;dCs;dTs;dCs;InaTs;InaTs;InaG- |
| | | | | | Sup |
| 115 | YEATS4- | ATTCGGCCATTC | YEATS4 | human | InaAs;InaTs;InaTs;dCs;dGs;dGs;dCs;d |
| | 02 m08 | TCT | | | Cs;dAs;dTs;dTs;dCs;InaTs;InaCs;InaT- |
| | | | | | Sup |
| 116 | YEATS4- | CCCGCCGGAGT | YEATS4 | human | InaCs;InaCs;InaCs;dGs;dCs;dCs;dGs;d |
| | 03 m08 | CAGG | | | Gs;dAs;dGs;dTs;dCs;InaAs;InaGs;InaG- |
| | | | | | Sup |
| 117 | YEATS4- | ATATGGAGGTT | YEATS4 | human | InaAs;InaTs;InaAs;dTs;dGs;dGs;dAs;d |
| | 04 m08 | TAGT | | | Gs;dGs;dTs;dTs;dTs;InaAs;InaGs;InaT- |
| | | | | | Sup |
| 118 | YEATS4- | TCGAATTCACCC | YEATS4 | human | InaTs;InaCs;InaGs;dAs;dAs;dTs;dTs;d |
| | 05 m08 | CAT | | | Cs;dAs;dCs;dCs;dCs;InaCs;InaAs;InaT- |
| | | | | | Sup |
| 119 | YEATS4- | TTCGAATTCACC | YEATS4 | human | InaTs;InaTs;InaCs;dGs;dAs;dAs;dTs;d |
| | 06 m08 | CCA | | | Ts;dCs;dAs;dCs;dCs;InaCs;InaCs;InaA- |
| | | | | | Sup |
| 120 | YEATS4- | TGACGAGATGT | YEATS4 | human | InaTs;InaGs;InaAs;dCs;dGs;dAs;dGs;d |
| | 07 m08 | TGTC | | | As;dTs;dGs;dTs;dTs;InaGs;InaTs;InaC- |
| | | | | | Sup |
| 121 | YEATS4- | TAGCTGACGAG | YEATS4 | human | InaTs;InaAs;InaGs;dCs;dTs;dGs;dAs;d |
| | 08 m08 | ATGT | | | Cs;dGs;dAs;dGs;dAs;InaTs;InaGs;InaT- |
| | | | | | Sup |
| 122 | YEATS4- | AGTTTCACGACT | YEATS4 | human | InaAs;InaGs;InaTs;dTs;dTs;dCs;dAs;d |
| | 09 m08 | TGC | | | Cs;dGs;dAs;dCs;dTs;InaTs;InaGs;InaC- |
| | | | | | Sup |
| 123 | ACTL6A- | GGCAACAAAGC | ACTL6A | human | InaGs;InaGs;InaCs;dAs;dAs;dCs;dAs;d |
| | 01 m08 | GGCG | | | As;dAs;dGs;dCs;dGs;InaGs;InaCs;Ina |
| | | | | | G-Sup |
| 124 | ACTL6A- | ATCGCCATCTAT | ACTL6A | human | InaAs;InaTs;InaCs;dGs;dCs;dCs;dAs;d |
| | 02 m08 | TTC | | | Ts;dCs;dTs;dAs;dTs;InaTs;InaTs;InaC- |
| | | | | | Sup |
| 125 | ACTL6A- | GAACGACCATT | ACTL6A | human | InaGs;InaAs;InaAs;dCs;dGs;dAs;dCs;d |
| | 03 m08 | AGCA | | | Cs;dAs;dTs;dTs;dAs;InaGs;InaCs;InaA- |
| | | | | | Sup |
| 126 | ACTL6A- | GCCCAGTAGAA | ACTL6A | human | InaGs;InaCs;InaCs;dCs;dAs;dGs;dTs;d |
| | 04 m08 | CGAC | | | As;dGs;dAs;dAs;dCs;InaGs;InaAs;InaC- |
| | | | | | Sup |
| 127 | ACTL6A- | CATCGTGGACT | ACTL6A | human | InaCs;InaAs;InaTs;dCs;dGs;dTs;dGs;d |
| | 05 m08 | GGAA | | | Gs;dAs;dCs;dTs;dGs;InaGs;InaAs;InaA- |
| | | | | | Sup |
| 128 | ACTL6A- | TTTCAACCGCAT | ACTL6A | human | InaTs;InaTs;InaTs;dCs;dAs;dAs;dCs;d |
| | 06 m08 | ACT | | | Cs;dGs;dCs;dAs;dTs;InaAs;InaCs;InaT- |
| | | | | | Sup |
| 129 | ACTL6A- | GAGCTAAACCT | ACTL6A | human | InaGs;InaAs;InaGs;dCs;dTs;dAs;dAs;d |
| | 07 m08 | CCGT | | | As;dCs;dCs;dTs;dCs;InaCs;InaGs;InaT- |
| | | | | | Sup |
| 130 | ACTL6A- | GAGCCGCCAAT | ACTL6A | human | InaGs;InaAs;InaGs;dCs;dCs;dGs;dCs;d |
| | 08 m08 | CCAT | | | Cs;dAs;dAs;dTs;dCs;InaCs;InaAs;InaT- |
| | | | | | Sup |
| 131 | EID1-01 | AAATTCCTCGCC | EID1 | human | InaAs;InaAs;InaAs;dTs;dTs;dCs;dCs;d |
| | m08 | CTC | | | Ts;dCs;dGs;dCs;dCs;InaCs;InaTs;InaC- |
| | | | | | Sup |
| 132 | EID1-02 | CGTAGTCGTCCT | EID1 | human | InaCs;InaGs;InaTs;dAs;dGs;dTs;dCs;d |
| | m08 | CCC | | | Gs;dTs;dCs;dCs;dTs;InaCs;InaCs;InaC- |
| | | | | | Sup |
| 133 | EID1-03 | CTGAAACCCGC | EID1 | human | InaCs;InaTs;InaGs;dAs;dAs;dAs;dCs;d |
| | m08 | CATC | | | Cs;dCs;dGs;dCs;dCs;InaAs;InaTs;InaC- |
| | | | | | Sup |
| 134 | EID1-04 | AGCTCTTCGATA | EID1 | human | InaAs;InaGs;InaCs;dTs;dCs;dTs;dTs;d |
| | m08 | AAA | | | Cs;dGs;dAs;dTs;dAs;InaAs;InaAs;InaA- |
| | | | | | Sup |
| 135 | EID1-05 | TCGGTCAGACG | EID1 | human | InaTs;InaCs;InaGs;dGs;dTs;dCs;dAs;d |
| | m08 | AUG | | | Gs;dAs;dCs;dGs;dAs;InaTs;InaTs;InaG- |
| | | | | | Sup |
| 136 | EID1-06 | CTCATCACAGCC | EID1 | human | InaCs;InaTs;InaCs;dAs;dTs;dCs;dAs;d |
| | m08 | GAG | | | Cs;dAs;dGs;dCs;dCs;InaGs;InaAs;InaG- |
| | | | | | Sup |
| 137 | IDH1-01 | ACGATTCTCTAT | IDH1 | human | InaAs;InaCs;InaGs;dAs;dTs;dTs;dCs;d |
| | m08 | GCC | | | Ts;dCs;dTs;dAs;dTs;InaGs;InaCs;InaC- |
| | | | | | Sup |
| 138 | IDH1-02 | TGGCATCACGA | IDH1 | human | InaTs;InaGs;InaGs;dCs;dAs;dTs;dCs;d |
| | m08 | TTCT | | | As;dCs;dGs;dAs;dTs;InaTs;InaCs;InaT- |
| | | | | | Sup |
| 139 | IDH1-03 | TCAATTGACTTA | IDH1 | human | InaTs;InaCs;InaAs;dAs;dTs;dTs;dGs;d |
| | m08 | TCT | | | As;dCs;dTs;dTs;dAs;InaTs;InaCs;InaT- |
| | | | | | Sup |
| 140 | IDH1-04 | ACGCCCATCAT | IDH1 | human | InaAs;InaCs;InaGs;dCs;dCs;dCs;dAs;d |
| | m08 | ATTT | | | Ts;dCs;dAs;dTs;dAs;InaTs;InaTs;InaT- |
| | | | | | Sup |
| 141 | IDH1-05 | TGTCTTTAAAAC | IDH1 | human | InaTs;InaGs;InaTs;dCs;dTs;dTs;dTs;dA |
| | m08 | GCC | | | s;dAs;dAs;dAs;dCs;InaGs;InaCs;InaC- |
| | | | | | Sup |
| 142 | IDH1-06 | TTATCAAGCTTT | IDH1 | human | InaTs;InaTs;InaAs;dTs;dCs;dAs;dAs;d |
| | m08 | GCT | | | Gs;dCs;dTs;dTs;dTs;InaGs;InaCs;InaT- |
| | | | | | Sup |
| 143 | JAK2-01 | GTCATCGTAAG | JAK2 | human | InaGs;InaTs;InaCs;dAs;dTs;dCs;dGs;d |
| | m08 | GCAG | | | Ts;dAs;dAs;dGs;dGs;InaCs;InaAs;InaG- |
| | | | | | Sup |
| 144 | JAK2-02 | GGATCTTTGCTC | JAK2 | human | InaGs;InaGs;InaAs;dTs;dCs;dTs;dTs;d |
| | m08 | GAA | | | Ts;dGs;dCs;dTs;dCs;InaGs;InaAs;InaA- |
| | | | | | Sup |
| 145 | JAK2-03 | TAGTCTTGGATC | JAK2 | human | InaTs;InaAs;InaGs;dTs;dCs;dTs;dTs;d |
| | m08 | -ITT | | | Gs;dGs;dAs;dTs;dCs;InaTs;InaTs;InaT- |
| | | | | | Sup |
| 146 | JAK2-04 | TGCGAAATCTG | JAK2 | human | InaTs;InaGs;InaCs;dGs;dAs;dAs;dAs;d |
| | m08 | TACC | | | Ts;dCs;dTs;dGs;dTs;InaAs;InaCs;InaC- |
| | | | | | Sup |
| 147 | JAK2-05 | TGAATTCCACC | JAK2 | human | InaTs;InaGs;InaAs;dAs;dTs;dTs;dCs;d |
| | m08 | GTTT | | | Cs;dAs;dCs;dCs;dGs;InaTs;InaTs;InaT- |
| | | | | | Sup |
| 148 | JAK2-06 | ATCGCAATATA | JAK2 | human | InaAs;InaTs;InaCs;dGs;dCs;dAs;dAs;d |
| | m08 | ACTG | | | Ts;dAs;dTs;dAs;dAs;InaCs;InaTs;InaG- |
| | | | | | Sup |
| 149 | JAK2-07 | TGACATTTTCTC | JAK2 | human | InaTs;InaGs;InaAs;dCs;dAs;dTs;dTs;d |
| | m08 | GCT | | | Ts;dTs;dCs;dTs;dCs;InaGs;InaCs;InaT- |
| | | | | | Sup |
| 150 | JAK2-08 | TCATACCGGCA | JAK2 | human | InaTs;InaCs;InaAs;dTs;dAs;dCs;dCs;d |
| | m08 | CATC | | | Gs;dGs;dCs;dAs;dCs;InaAs;InaTs;InaC- |
| | | | | | Sup |
| 151 | JAK2-09 | GTCTCGTAAACT | JAK2 | human | InaGs;InaTs;InaCs;dTs;dCs;dGs;dTs;d |
| | m08 | TCC | | | As;dAs;dAs;dCs;dTs;InaTs;InaCs;InaC- |
| | | | | | Sup |
| 152 | JAK2-10 | TGATCTATCCGT | JAK2 | human | InaTs;InaGs;InaAs;dTs;dCs;dTs;dAs;d |
| | m08 | TCT | | | Ts;dCs;dCs;dGs;dTs;InaTs;InaCs;InaT- |
| | | | | | Sup |
| 153 | KAT2A-01 | AGGTCGAGCCG | KAT2A | human | InaAs;InaGs;InaGs;dTs;dCs;dGs;dAs;d |
| | m08 | GATC | | | Gs;dCs;dCs;dGs;dGs;InaAs;InaTs;InaC- |
| | | | | | Sup |
| 154 | KAT2A-02 | CCGGACTTGCG | KAT2A | human | InaCs;InaCs;InaGs;dGs;dAs;dCs;dTs;d |
| | m08 | CCTT | | | Ts;dGs;dCs;dGs;dCs;InaCs;InaTs;InaT- |
| | | | | | Sup |
| 155 | KAT2A-03 | TGAGAGCTCGA | KAT2A | human | InaTs;InaGs;InaAs;dGs;dAs;dGs;dCs;d |
| | m08 | ACAT | | | Ts;dCs;dGs;dAs;dAs;InaCs;InaAs;InaT- |
| | | | | | Sup |
| 156 | KAT2A-04 | CACGGAGCCGC | KAT2A | human | InaCs;InaAs;InaCs;dGs;dGs;dAs;dGs;d |
| | m08 | TTGG | | | Cs;dCs;dGs;dCs;dTs;InaTs;InaGs;InaG- |
| | | | | | Sup |
| 157 | KAT2A-05 | CATTGACCAGC | KAT2A | human | InaCs;InaAs;InaTs;dTs;dGs;dAs;dCs;d |
| | m08 | TCCA | | | Cs;dAs;dGs;dCs;dTs;InaCs;InaCs;InaA- |
| | | | | | Sup |
| 158 | KAT2A-06 | GGCGATATACT | KAT2A | human | InaGs;InaGs;InaCs;dGs;dAs;dTs;dAs;d |
| | m08 | CCTT | | | Ts;dAs;dCs;dTs;dCs;InaCs;InaTs;InaT- |
| | | | | | Sup |
| 159 | KAT2A-07 | CACCGATGACC | KAT2A | human | InaCs;InaAs;InaCs;dCs;dGs;dAs;dTs;d |
| | m08 | CGCC | | | Gs;dAs;dCs;dCs;dCs;InaGs;InaCs;InaC- |
| | | | | | Sup |
| 160 | KAT2A-08 | CCATCAGCGTC | KAT2A | human | InaCs;InaCs;InaAs;dTs;dCs;dAs;dGs;d |
| | m08 | GCTC | | | Cs;dGs;dTs;dCs;dGs;InaCs;InaTs;InaC- |
| | | | | | Sup |
| 161 | KAT2A-09 | CTGTTTGCGCTC | KAT2A | human | InaCs;InaTs;InaGs;dTs;dTs;dTs;dGs;d |
| | m08 | AAT | | | Cs;dGs;dCs;dTs;dCs;InaAs;InaAs;InaT- |
| | | | | | Sup |
| 162 | KAT2A-10 | GGCGATGACCC | KAT2A | human | InaGs;InaGs;InaCs;dGs;dAs;dTs;dGs;d |
| | m08 | GCTG | | | As;dCs;dCs;dCs;dGs;InaCs;InaTs;InaG- |
| | | | | | Sup |
| 163 | PRKCD-01 | CATGGTCGGCT | PRKCD | human | InaCs;InaAs;InaTs;dGs;dGs;dTs;dCs;d |
| | m08 | TCTT | | | Gs;dGs;dCs;dTs;dTs;InaCs;InaTs;InaT- |
| | | | | | Sup |
| 164 | PRKCD-02 | TGCGCATAGAC | PRKCD | human | InaTs;InaGs;InaCs;dGs;dCs;dAs;dTs;d |
| | m08 | TGTT | | | As;dGs;dAs;dCs;dTs;InaGs;InaTs;InaT- |
| | | | | | Sup |
| 165 | PRKCD-03 | GGTGGCGATAA | PRKCD | human | InaGs;InaGs;InaTs;dGs;dGs;dCs;dGs;d |
| | m08 | ACTC | | | As;dTs;dAs;dAs;dAs;InaCs;InaTs;InaC- |
| | | | | | Sup |
| 166 | PRKCD-04 | ATCTTGTCGATG | PRKCD | human | InaAs;InaTs;InaCs;dTs;dTs;dGs;dTs;d |
| | m08 | CAT | | | Cs;dGs;dAs;dTs;dGs;InaCs;InaAs;InaT- |
| | | | | | Sup |
| 167 | PRKCD-05 | TGTTGAAGCGT | PRKCD | human | InaTs;InaGs;InaTs;dTs;dGs;dAs;dAs;d |
| | m08 | TCTT | | | Gs;dCs;dGs;dTs;dTs;InaCs;InaTs;InaT- |
| | | | | | Sup |
| 168 | PRKCD-06 | CGATGTTGAAG | PRKCD | human | InaCs;InaGs;InaAs;dTs;dGs;dTs;dTs;d |
| | m08 | CGTT | | | Gs;dAs;dAs;dGs;dCs;InaGs;InaTs;InaT- |
| | | | | | Sup |
| 169 | PRKCD-07 | AAGCGGCCTTT | PRKCD | human | InaAs;InaAs;InaGs;dCs;dGs;dGs;dCs;d |
| | m08 | GTCC | | | Cs;dTs;dTs;dTs;dGs;InaTs;InaCs;InaC- |
| | | | | | Sup |
| 170 | PRKCD-08 | TAGAGTTCAAA | PRKCD | human | InaTs;InaAs;InaGs;dAs;dGs;dTs;dTs;d |
| | m08 | GCGG | | | Cs;dAs;dAs;dAs;dGs;InaCs;InaGs;InaG- |
| | | | | | Sup |
| 171 | PRKCD-09 | CCCCGAAAGAC | PRKCD | human | InaCs;InaCs;InaCs;dCs;dGs;dAs;dAs;d |
| | m08 | CACC | | | As;dGs;dAs;dCs;dCs;InaAs;InaCs;InaC- |
| | | | | | Sup |
| 172 | PRKCD-10 | CACGGATGGAC | PRKCD | human | InaCs;InaAs;InaCs;dGs;dGs;dAs;dTs;d |
| | m08 | TCGA | | | Gs;dGs;dAs;dCs;dTs;InaCs;InaGs;InaA- |
| | | | | | Sup |
| 173 | PRKCD-11 | AGTCGATGAGG | PRKCD | human | InaAs;InaGs;InaTs;dCs;dGs;dAs;dTs;d |
| | m08 | TTCT | | | Gs;dAs;dGs;dGs;dTs;InaTs;InaCs;InaT- |
| | | | | | Sup |
| 174 | TNFSF9- | GTCAGAGGCGT | TNFSF9 | human | InaGs;InaTs;InaCs;dAs;dGs;dAs;dGs;d |
| | 01 m08 | ATTC | | | Gs;dCs;dGs;dTs;dAs;InaTs;InaTs;InaC- |
| | | | | | Sup |
| 175 | TNFSF9- | AGCAGCCCCGC | TNFSF9 | human | InaAs;InaGs;InaCs;dAs;dGs;dCs;dCs;d |
| | 02 m08 | GACC | | | Cs;dCs;dGs;dCs;dGs;InaAs;InaCs;InaC- |
| | | | | | Sup |
| 176 | TNFSF9- | GACGGCGCAGG | TNFSF9 | human | InaGs;InaAs;InaCs;dGs;dGs;dCs;dGs;d |
| | 03 m08 | CGGC | | | Cs;dAs;dGs;dGs;dCs;InaGs;InaGs;InaC- |
| | | | | | Sup |
| 177 | TNFSF9- | CTGAGCCCTCG | TNFSF9 | human | InaCs;InaTs;InaGs;dAs;dGs;dCs;dCs;d |
| | 04 m08 | CCGG | | | Cs;dTs;dCs;dGs;dCs;InaCs;InaGs;InaG- |
| | | | | | Sup |
| 178 | TNFSF9- | GGTCCACGGTC | TNFSF9 | human | InaGs;InaGs;InaTs;dCs;dCs;dAs;dCs;d |
| | 05 m08 | AAAG | | | Gs;dGs;dTs;dCs;dAs;InaAs;InaAs;InaG- |
| | | | | | Sup |
| 179 | TNFSF9- | AAACCGAAGGC | TNFSF9 | human | InaAs;InaAs;InaAs;dCs;dCs;dGs;dAs;d |
| | 06 m08 | CGAG | | | As;dGs;dGs;dCs;dCs;InaGs;InaAs;Ina |
| | | | | | G-Sup |
| 180 | TNFSF9- | AGGTGCAGCAA | TNFSF9 | human | InaAs;InaGs;InaGs;dTs;dGs;dCs;dAs;d |
| | 07 m08 | GCGG | | | Gs;dCs;dAs;dAs;dGs;InaCs;InaGs;Ina |
| | | | | | G-Sup |
| 181 | TNFSF9- | GTCACCCGGAA | TNFSF9 | human | InaGs;InaTs;InaCs;dAs;dCs;dCs;dCs;d |
| | 08 m08 | GAGT | | | Gs;dGs;dAs;dAs;dGs;InaAs;InaGs;Ina |
| | | | | | T-Sup |
| 182 | TNFSF9- | AGTAGGATTCG | TNFSF9 | human | InaAs;InaGs;InaTs;dAs;dGs;dGs;dAs;d |
| | 09 m08 | GACT | | | Ts;dTs;dCs;dGs;dGs;InaAs;InaCs;InaT- |
| | | | | | Sup |

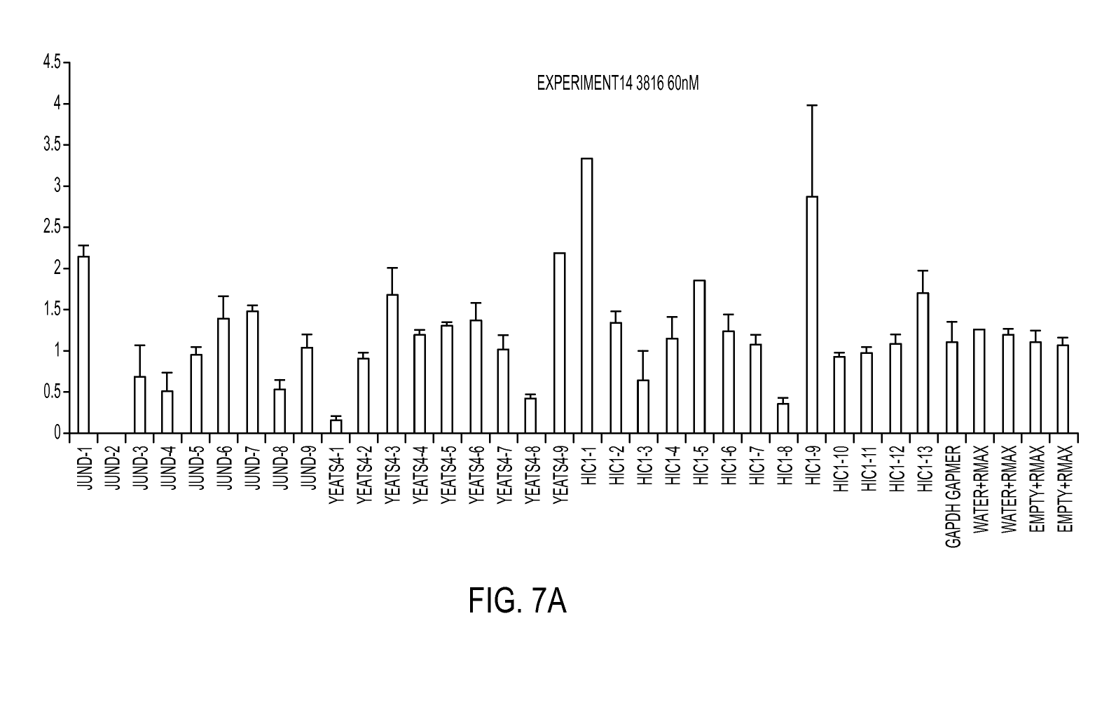

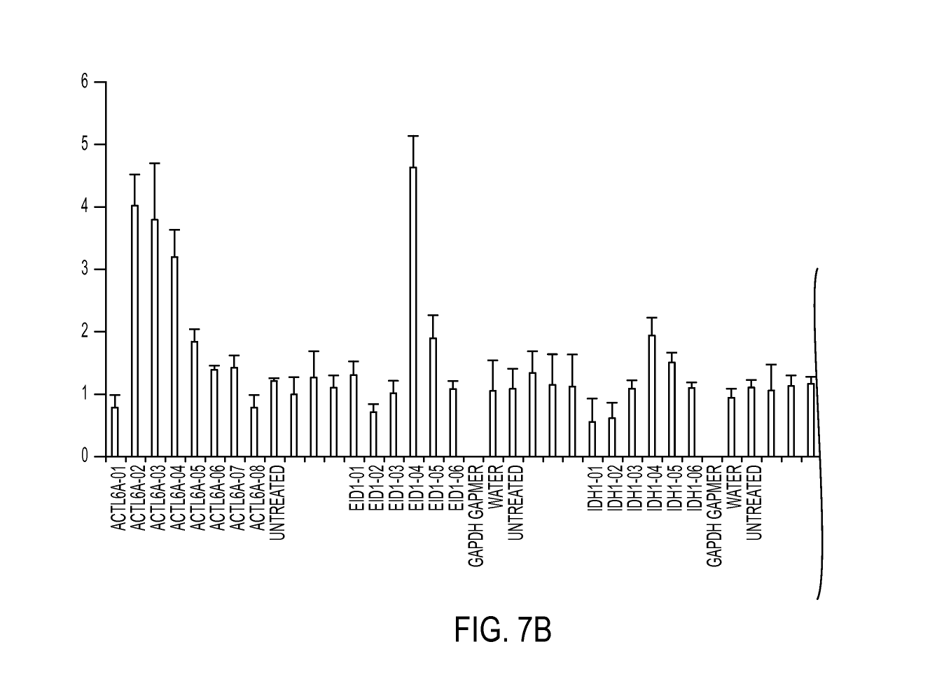

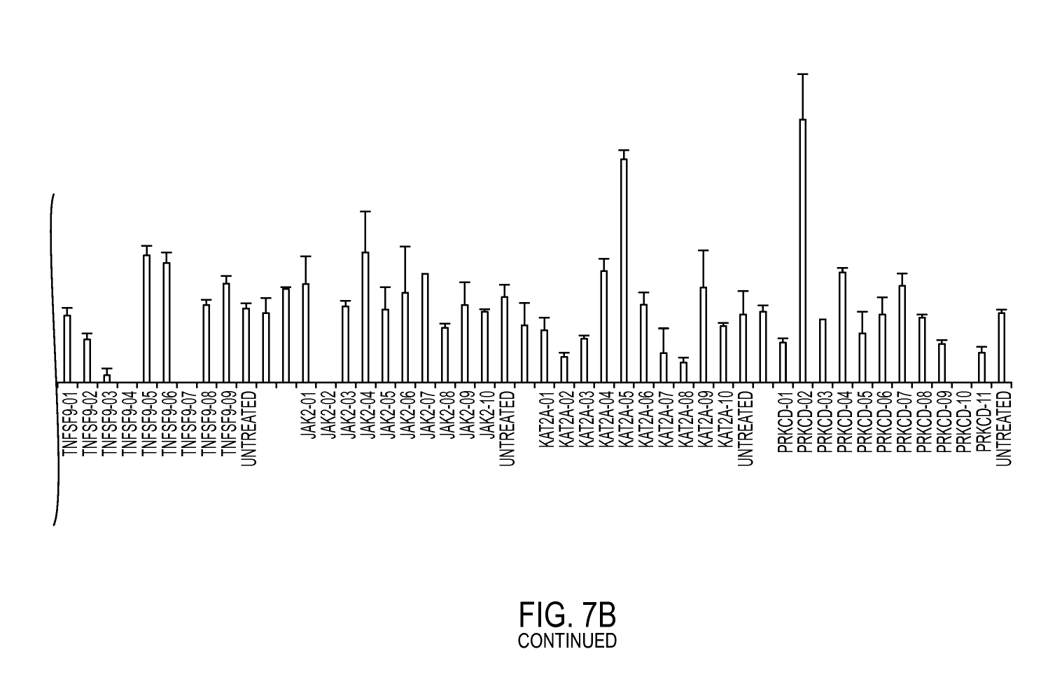

The gapmers were screened in GM03816 cells via lipofection at 60 nM concentration. In general, at least one gapmer from each gene caused upregulation of FXN mRNA (FIGS. 7A and B).

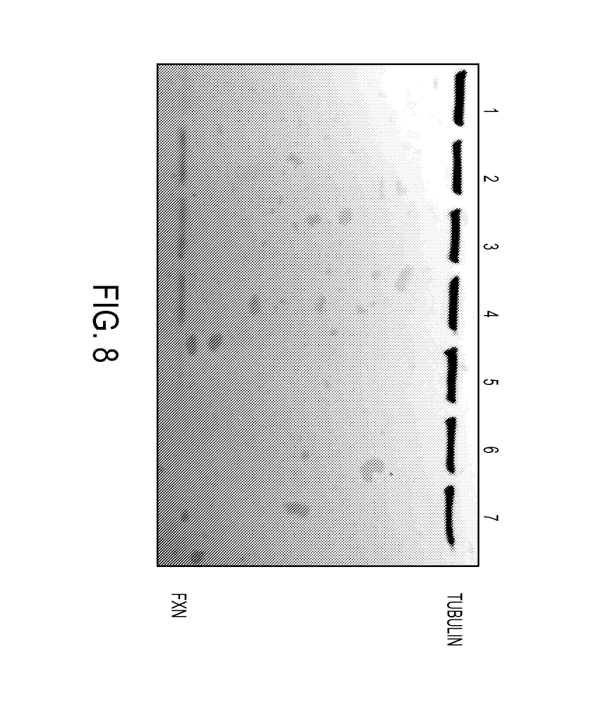

Next, a Western blot with the Abcam ab48281 FXN and Abcam ab125267 tubulin antibodies were run using treated and untreated GM03816 lysates. Several strong upregulation oligos were identified, including ACTL6A-3, JUND-1, and PRKCD-2 (FIG. 8).

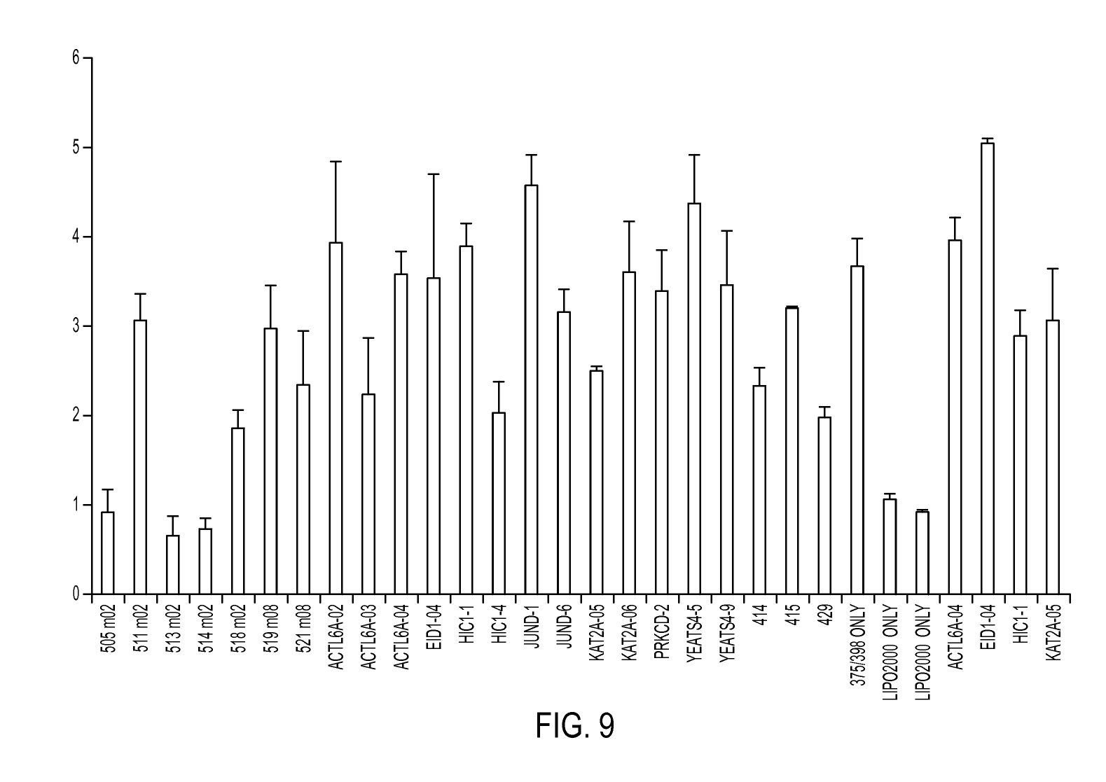

Subsequently, various oligos targeting ACTL6A, EID1, HIC1, JUND, KAT2A, PRKCD, and YEATS4 were screened in differentiated myotubes for FXN mRNA levels. Measurements were taken 4 days after transfection. Several of the oligos showed upregulation of FXN mRNA, including ACTL6A-02, 03, 04, EID1-04, HIC1-1, JUND-1, JUND-6, KAT2A-05, KAT2A-06, PRKCD-2, YEATS4-5, and YEATS4-9 (FIG. 9).

These results demonstrate that oligos targeting epigenetic silencers of FXN can be used to upregulate FXN levels.

### Example 5

### Data for SUV39H1 and Tip60

SUV39H1 and Tip60, as well as HDAC1, HDAC2, HDAC3, and G9a, were tested as potential drivers of FRDA epigenetic silencing. ChIP for candidate chromatin modifying enzymes that may be responsible for GAA-repeat associated silencing was done in diseased (GM03816 fibroblasts, GM16209 lymphoblasts) and normal (GM15851 lymphoblasts) cells. The antibodies used were HDAC1 ab46985, HDAC2 ab51832, HDAC3 ab96005, G9a ab40542, SUV39H1 ab12405, Tip60 ab23886, H3K27me3 ab6002, and H3K9me3 ab8898.

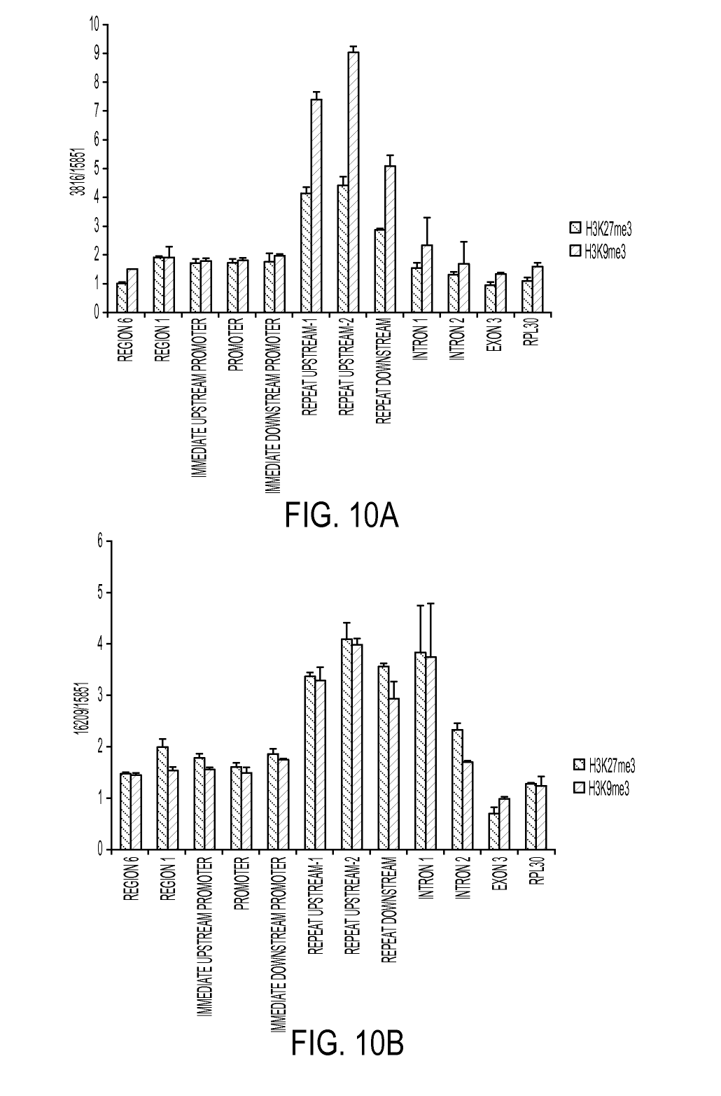

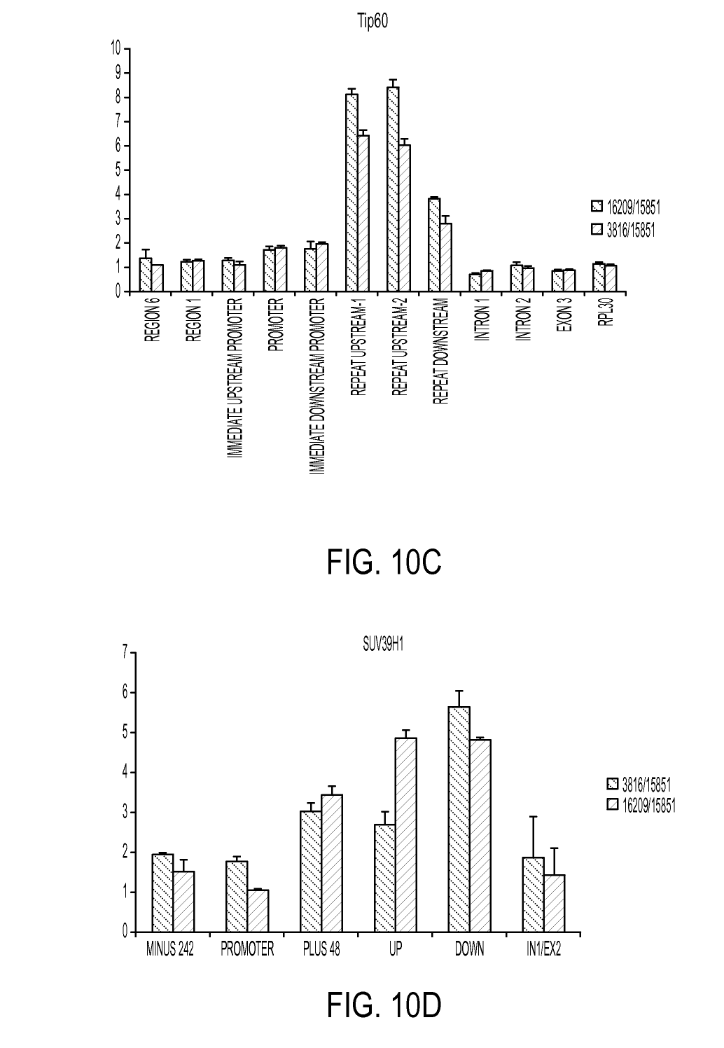

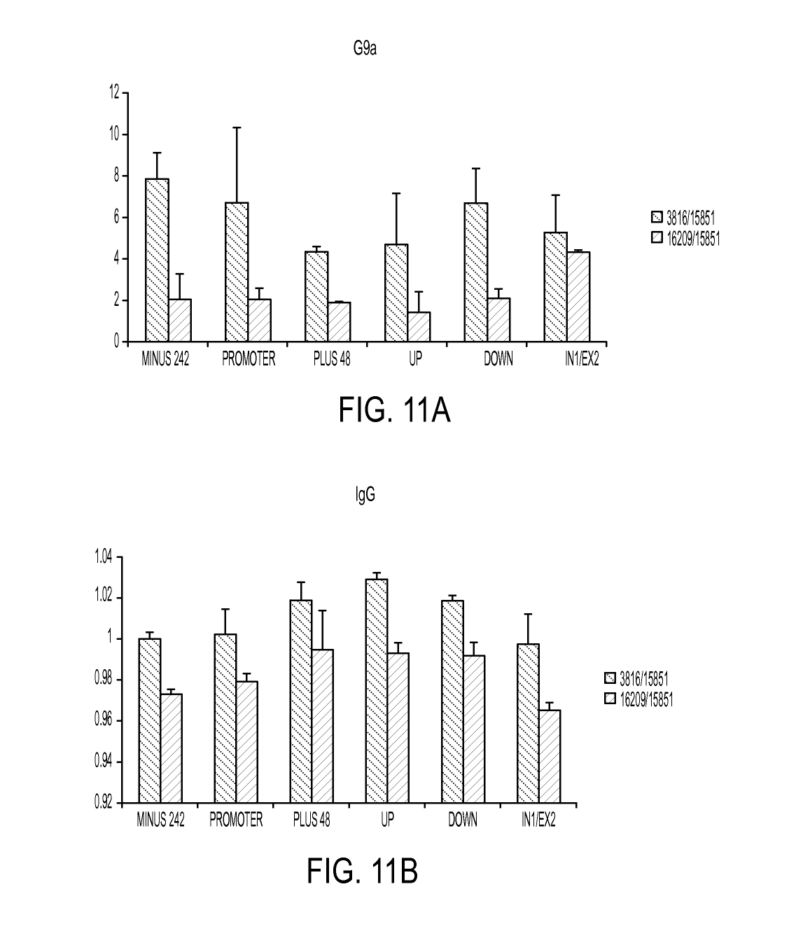

Enrichment obtained in each diseased line was normalized to the normal line levels. H3K27me3 and H3K9me3 enrichment patterns in disease tissue was at least partly mirrored by Tip60 and SUV39H1 patterns (FIG. 10A-D). Enrichment patterns for G9a (an H3K9 methyltranserase) were also measured (FIG. 11A). Enrichment of IgG was used as a control (FIG. 11B). These data indicate that Tip60 and SUV39H1 may be involved in the FRDA epigenetic silencing.

Without further elaboration, it is believed that one skilled in the art can, based on the description provided herein, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method for increasing FXN expression in a cell, the method comprising:
delivering to a cell an oligonucleotide that inhibits expression or activity of a negative epigenetic regulator of FXN, thereby increasing FXN expression in the cell, wherein, prior to delivering, the cell has a lower level of FXN expression compared to an appropriate control level of FXN expression.

2. The method of claim 1, wherein, prior to delivering, the cell has a higher level of histone H3 K27 or K9 methylation at the FXN gene compared with an appropriate control level of histone H3 K27 or K9 methylation.

3. The method of claim 1, wherein the cell comprises an FXN gene encoding in its first intron a GAA repeat of between 10-2000 units.

4. The method of claim 1, wherein the cell is obtained from or present in a subject having Friedreich's ataxia.

5. The method of claim 1, wherein the negative epigenetic regulator of FXN is a component of a histone H2A acetylation pathway, a NuA4 histone acetyltransferase complex, a protein amino acid acetylation pathway, a histone acetylation pathway, a protein amino acid acylation pathway, a H4/H2A histone acetyltransferase complex, a nucleotide binding pathway, a histone H4 acetylation pathway, a histone acetyltransferase complex, or an insulin receptor substrate binding pathway.

6. The method of claim 5, wherein
(i) the component of the histone H2A acetylation pathway is MEAF6, YEATS4, ACTL6A, or DMAP1; or
(ii) the component of the NuA4 histone acetyltransferase complex is MEAF6, YEATS4, ACTL6A, or DMAP1; or
(iii) the component of the protein amino acid acetylation pathway is KAT2A, MEAF6, YEATS4, TADA3, ACTL6A, or DMAP1; or
(iv) the component of the histone acetylation pathway is KAT2A, MEAF6, YEATS4, TADA3, ACTL6A, or DMAP1; or
(v) the component of the protein amino acid acylation pathway is KAT2A, MEAF6, YEATS4, TADA3, ACTL6A, or DMAP1; or
(vi) the component of the H4/H2A histone acetyltransferase complex is MEAF6, YEATS4, ACTL6A, or DMAP1; or
(vii) the component of the nucleotide binding pathway is MEF2D, PRKDC, IDH1, ACTL6A, JAK2, CFTR, SPEN, or PRKCD; or
(viii) the component of the histone H4 acetylation pathway is MEAF6, YEATS4, ACTL6A, or DMAP1; or
(ix) the component of the histone acetyltransferase complex is KAT2A, MEAF6, YEATS4, TADA3, ACTL6A, or DMAP1; or
(x) the component of the insulin receptor substrate binding pathway is JAK2 or PRKCD.

7. The method of claim 1, wherein the negative epigenetic regulator of FXN is TNFSF9, JUND, HIC1, PRKCD, JAK2, EID1, CFTR, TADA3, MYBL2, KAT2A, IDH1, SUMO1, SPEN, PRKDC, KIR2DL4, APC, MEF2D, a component of the NuA4 Histone Acetyltransferase Complex, or a histone-lysine N-methyltransferase.

8. The method of claim 1, wherein the negative epigenetic regulator of FXN is a component of the NuA4 Histone Acetyltransferase Complex.

9. The method of claim 8, wherein the component of the NuA4 Histone Acetyltransferase Complex is YEATS4, Eaf1, TRRAP, P400, EPC1, DMAP1, Tip60, MRG15, MRGX, MORF4, ACTB, ACTL6A, ING1, ING2, ING3, ING4, ING5, RUVBL1, RUVBL2, AF9, ENL, or MEAF6.

10. The method of claim 9, wherein the component of the NuA4 Histone Acetyltransferase Complex is YEATS4, ACTL6A, DMAP1, or MEAF6.

11. The method of claim 9, wherein the component of the NuA4 Histone Acetyltransferase Complex is YEATS4.

12. The method of claim 1, wherein the negative epigenetic regulator of FXN is a histone-lysine N-methyltransferase.

13. The method of claim 12, wherein the histone-lysine N-methyltransferase is SUV39H1, SUV39H2, SETDB1, PRDM2, G9A and EHMT1.

14. The method of claim 12, wherein the histone-lysine N-methyltransferase is SUV39H1.

15. The method of claim 1, wherein the negative epigenetic regulator of FXN is YEATS4, HIC1, JUND, TNFSF9, PRKCD, KAT2A, JAK2, IDH1, EID1, or ACTL6A.

16. The method of claim 15, wherein the oligonucleotide comprises a sequence as set for in Table 4.

17. The method of claim 15, wherein the oligonucleotide comprises a sequence as set for in Table 12.

18. The method of claim 1, wherein the negative epigenetic regulator of FXN is the product of a gene listed in Table 6 and/or 9 that has a fold change greater than 1.25.

19. The method of claim 1, wherein presence of the oligonucleotide in the cell results in decreased levels of mRNA of the negative epigenetic regulator of FXN.

20. The method of claim 1, wherein the appropriate control is a level of FXN in a cell from a subject or in cells from a population of subjects that do not have Friedreich's ataxia.

21. The method of claim 1, wherein the oligonucleotide is a gapmer, a mixmer, an siRNA, a single stranded RNA, a single stranded DNA, an aptamer, or a ribozyme.

22. The method of claim 1, wherein the oligonucleotide comprises at least one modified nucleotide or internucleoside linkage.

23. The method of claim 22, wherein the oligonucleotide is a single stranded oligonucleotide.

24. The method of claim 23, wherein the single stranded oligonucleotide comprises the sequence 5′-X-Y-Z-3′, wherein X comprises 1-5 modified nucleotides, Y comprises at least 6 unmodified nucleotides, and Z comprises 1-5 modified nucleotides.

25. The method of claim 24, wherein the X comprises 1-5 LNAs, Y comprises at least 6 DNAs, and Z comprises 1-5 LNAs.

26. The method of claim 1, wherein the method further comprises:
delivering to the cell a second oligonucleotide that inhibits expression or activity of a second negative epigenetic regulator of FXN.

27. The method of claim 26, wherein the second negative epigenetic regulator of FXN is TNFSF9, JUND, HIC1, PRKCD, JAK2, EID1, CFTR, TADA3, MYBL2, KAT2A, IDH1, SUMO1, SPEN, PRKDC, KIR2DL4, APC, MEF2D, a component of the NuA4 Histone Acetyltransferase Complex, or a histone-lysine N-methyltransferase.

28. An oligonucleotide comprising a sequence as set forth in Table 4.

29. An oligonucleotide comprising a sequence as set forth in Table 12.

30. The oligonucleotide of claim 28, wherein the oligonucleotide comprises at least one modified nucleotide or internucleoside linkage.

31. The oligonucleotide of claim 28, wherein the oligonucleotide is 50 nucleotides or fewer in length.

32. The oligonucleotide of claim 31, wherein the oligonucleotide consists of a sequence as set forth in Table 4.

33. The oligonucleotide of claim 31, wherein the oligonucleotide consists of a sequence as set forth in Table 12.

34. A method for increasing FXN expression in a cell, the method comprising:
delivering to a cell an expression vector that is engineered to express a positive epigenetic regulator of FXN, thereby increasing FXN expression in the cell, wherein, prior to delivering, the cell has a lower level of FXN expression compared to an appropriate control level of FXN expression.

35. The method of claim 34, wherein positive epigenetic regulator of FXN is the product of a gene listed in Table 6 and/or 9 that has a fold change less than or equal to 0.75.

36. A method for modulating FXN expression in a cell, the method comprising delivering to a cell an effective amount of a histone-lysine N-methyltransferase inhibitor.

37. The method of claim 36, wherein the inhibitor is listed in Table 2 or otherwise disclosed herein.

38. A method for modulating FXN expression in a cell, the method comprising delivering to a cell an effective amount of an agent listed in Table 10 or 11 that modulates FXN expression.

39. The method of claim 38, wherein delivery of the agent results in an increase in FXN expression in the cell.
